(19) United States
(12) Patent Application Publication
(10) Pub. No.: US 2023/0033663 A1
(43) Pub. Date: Feb. 2, 2023

(54) METHODS AND COMPOSITIONS FOR TREATING ATOPIC DERMATITIS

(71) Applicant: Second Genome, Inc., Brisbane, CA (US)

(72) Inventor: Jayamary Divya Ravichandar, Brisbane, CA (US)

(21) Appl. No.: 17/758,773

(22) PCT Filed: Jan. 15, 2021
(86) PCT No.: PCT/US2021/013697

§ 371 Date: Jul. 13, 2022

Related U.S. Application Data
(60) Provisional application No. 62/962,856, filed on Jan. 17, 2020.

(51) Int. Cl. A61K 35/741; A61P 17/00; G01N 33/569

(52) U.S. Cl. CPC A61K 35/741; A61P 17/00; G01N 33/56911; G01N 2800/202

(57) ABSTRACT

Methods and compositions are provided herein for treating atopic dermatitis in a subject, using one or more bacterial strains such as Brevundimonas nasdae W1-2B, Capnocytophaga sputigena 4, Moraxella sp. LMG 5131, Neisseria elongata ATCC 29315, Staphylococcus felis GD521, Staphylococcus sciuri SC116, or Streptococcus intermedius 393.

### CROSS-REFERENCE TO RELATED APPLICATION

This application claims priority to U.S. Provisional Patent Application Ser. No. 62/962,856, filed Jan. 17, 2020; the entire contents of which are herein incorporated by reference.

### DESCRIPTION OF THE TEXT FILE SUBMITTED ELECTRONICALLY

The contents of the text file submitted electronically herewith are incorporated herein by reference in their entirety: A computer readable format copy of the Sequence Listing filename: SequenceListing.txt, date created, Jan. 17, 2020, file size 275 kilobytes.

### TECHNICAL FIELD

The present disclosure is related to bacterial strains and compositions thereof, and using such bacterial strains and compositions thereof for treating atopic dermatitis in a subject.

### BACKGROUND

The human microbiome comprises a diverse array of microorganisms, primarily prokaryotes, which play a significant role in the health of the host organism. The complexity of the microbiome, in terms of both its population makeup and composite function, has recently become an intense area of study as research increasingly shows that manipulation of the microbiome can provide health benefits and may be effective in treating a number of diseases and disorders. Currently, a number of probiotics are marketed which contain live bacteria and yeast and are believed to augment the benefits of these microbes which naturally occur in the human body. Increasingly, live biotherapeutic products (LBPs) are being developed for controlled clinical studies and regulatory approval in the treatment of disease.

Commensal skin microbes can control adaptive skin immune homeostasis, e.g., they can protect against the overgrowth of pathogens (see, e.g., Fyhrquist et al. Nat Commun. 2019; 10: 4703). Furthermore, commensal skin microbes are associated with disease flares in diseases such as atopic dermatitis. Atopic dermatitis prevalence is increasing impacting approximately 10-20% of children and approximately 1-3% of adults (Nutten S. Ann Nutr Metab. 2015; 66(Suppl 1):8-16). Microbe-based therapies could aid in the prevention and treatment of atopic dermatitis.

### SUMMARY

Provided herein are methods and compositions for treating a subject in need thereof.

Also provided herein are methods for identifying a subject as having atopic dermatitis that include (a) identifying a subject having a sample that has (i) an increased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) bacterial species selected from: Corynebacterium genitalium, Corynebacterium matruchotii, Corynebacterium striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; and/or (ii) a decreased level of one or more (e.g., two or more, three or more, four or more, five or more, six or more, or seven) bacterial species selected from: Brevundimonas nasdae, Capnocytophaga sputigena, Moraxella sp., Neisseria elongata, Staphylococcus felis, Staphylococcus sciuri, and Streptococcus intermedius; as having atopic dermatitis; or (b) identifying a subject having a sample that does not have (i) an increased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) bacterial species selected from: Corynebacterium genitalium, Corynebacterium matruchotii, Corynebacterium striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; or (ii) a decreased level of one or more (e.g., two or more, three or more, four or more, five or more, six or more, or seven) bacterial species selected from: Brevundimonas nasdae, Capnocytophaga sputigena, Moraxella sp., Neisseria elongata, Staphylococcus felis, Staphylococcus sciuri, and Streptococcus intermedius; as not having atopic dermatitis.

Also provided herein are methods for diagnosing a subject as having atopic dermatitis that include (a) diagnosing a subject having a sample that has (i) an increased level of one or more (e.g. two or more, three or more, four or more, five or more, six or more, seven or more, eight or more, nine or more, ten or more, eleven or more, twelve or more, thirteen or more, fourteen or more, fifteen or more, sixteen or more, or seventeen) bacterial species selected from: Corynebacterium genitalium, Corynebacterium matruchotii, Corynebacteriu striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; and/or (ii) a decreased level of one or more (e.g., two or more, three or more, four or more, five or more, six or more, or seven) bacterial species selected from: Brevundimonas nasdae, Capnocytophaga sputigena, Moraxella sp., Neisseria elongata, Staphylococcus felis, Staphylococcus sciuri, and Streptococcus intermedius; as having atopic dermatitis; or (b) diagnosing a subject having a sample that does not have (i) an increased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) bacterial species selected from: Corynebacterium genitalium, Corynebacterium matruchotii, Corynebacterium striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; or (ii) a decreased level of one or more (e.g., two or more, three or more, four or more, five or more, six or more, or seven) bacterial species selected from: Brevundimonas nasdae, Capnocytophaga sputigena, Moraxella sp., Neisseria elongata, Staphylococcus felis, Staphylococcus sciuri, and Streptococcus intermedius; as not having atopic dermatitis.

Also provided herein are methods of treating atopic dermatitis in a subject that include (a) administering an atopic dermatitis therapy to a subject determined to have a sample that has (i) an increased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) of: Corynebacterium genitalium, Corynebacterium matruchotii, Corynebacterium striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; and/or (ii) a decreased level of one or more (e.g., two or more, three or more, four or more, five or more, six or more, or seven) of: Brevundimonas nasdae, Capnocytophaga sputigena, Moraxella sp., Neisseria elongata, Staphylococcus felis, Staphylococcus sciuri, and Streptococcus intermedius; or (b) not administering an atopic dermatitis therapy to a subject determined not to have a sample that has (i) an increased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) of: Corynebacterium genitalium, Corynebacterium matruchotii, Corynebacterium striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; and/or (ii) a decreased level of one or more (e.g., two or more, three or more, four or more, five or more, six or more, or seven) of: Brevundimonas nasdae, Capnocytophaga sputigena, Moraxella sp., Neisseria elongata, Staphylococcus felis, Staphylococcus sciuri, and Streptococcus intermedius.

Also provided herein are methods of treating atopic dermatitis in a subject that include (a) administering a composition comprising an effective amount of a bacterial species selected from the group consisting of: Brevundimonas nasdae, Capnocytophaga sputigena, Moraxella sp., Neisseria elongata, Staphylococcus felis, Staphylococcus sciuri, and Streptococcus intermedius, and a combination thereof, as a monotherapy, or in conjunction with an atopic dermatitis therapy, to a subject determined to have a sample that has (i) an increased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) of: Corynebacterium genitalium, Corynebacterium matruchotii, Corynebacterium striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; and/or (ii) a decreased level of one or more (e.g., two or more, three or more, four or more, five or more, six or more, or seven) of: Brevundimonas nasdae, Capnocytophaga sputigena, Moraxella sp., Neisseria elongata, Staphylococcus felis, Staphylococcus sciuri, and Streptococcus intermedius; or (b) not administering a composition comprising an effective amount of a bacterial species selected from the group consisting of: Brevundimonas nasdae, Capnocytophaga sputigena, Moraxella sp., Neisseria elongata, Staphylococcus felis, Staphylococcus sciuri, and Streptococcus intermedius, and a combination thereof, to a subject determined not to have a sample that has (i) an increased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) of: Corynebacterium genitalium, Corynebacterium matruchotii, Corynebacterium striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; and/or (ii) a decreased level of one or more (e.g., two or more, three or more, four or more, five or more, six or more, or seven) of: Brevundimonas nasdae, Capnocytophaga sputigena, Moraxella, Neisseria elongata, Staphylococcus felis, Staphylococcus sciuri, and Streptococcus intermedius.

In some embodiments of any of the methods described herein, the method comprises detecting the level of one or more bacterial species in the sample from the subject.

In some embodiments of any of the methods described herein, the level of Corynebacterium genitalium, Corynebacterium matruchotii, Corynebacterium striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, or Turicella otitidis is increased in comparison to the same bacterial species in a reference sample.

In some embodiments of any of the methods described herein, the method comprises determining that the sample has (i) an increased level of two or more (e.g., three or more, four or more, five or more, six or more, seven or more, eight or more, nine or more, ten or more, eleven or more, twelve or more, thirteen or more, fourteen or more, fifteen or more, sixteen or more, or seventeen) of: Corynebacterium genitalium, Corynebacterium matruchotii, Corynebacterium striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; and/or (ii) a decreased level of two or more (e.g., three or more, four or more, five or more, six or more, or seven) of: Brevundimonas nasdae, Capnocytophaga sputigena, Moraxella sp., Neisseria elongata, Staphylococcus felis, Staphylococcus sciuri, and Streptococcus intermedius.

In some embodiments of any of the methods described herein, the method comprises determining that the sample has (i) an increased level of three or more (e.g., four or more, five or more, six or more, seven or more, eight or more, nine or more, ten or more, eleven or more, twelve or more, thirteen or more, fourteen or more, fifteen or more, sixteen or more, or seventeen) of: Corynebacterium genitalium, Corynebacterium matruchotii, Corynebacterium striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; and/or (ii) a decreased level of two or more (e.g., three or more, four or more, five or more, six or more, or seven) of: Brevundimonas nasdae, Capnocytophaga sputigena, Moraxella sp., Neisseria elongata, Staphylococcus felis, Staphylococcus sciuri, and Streptococcus intermedius.

In some embodiments of any of the methods described herein, the method comprises determining that the sample has (i) an increased level of four or more (e.g., five or more, six or more, seven or more, eight or more, nine or more, ten or more, eleven or more, twelve or more, thirteen or more, fourteen or more, fifteen or more, sixteen or more, or seventeen) of: Corynebacterium genitalium, Corynebacterium matruchotii, Corynebacterium striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; and/or (ii) a decreased level of two or more (e.g., three or more, four or more, five or more, six or more, or seven) of: Brevundimonas nasdae, Capnocytophaga sputigena, Moraxella sp., Neisseria elongata, Staphylococcus felis, Staphylococcus sciuri, and Streptococcus intermedius.

In some embodiments of any of the methods described herein, the atopic dermatitis therapy comprises a probiotic.

In some embodiments of any of the methods described herein, the atopic dermatitis therapy comprises vitamin D, an antihistamine, a corticosteroid, a calcineurin inhibitor, an immunosuppressant, a phosphodiesterase-4 inhibitor, an anti-interleukin-4 receptor (IL-4Rα) antibody, an antibiotic, phototherapy, or a combination thereof.

In some embodiments of any of the methods described herein, the corticosteroid comprises hydrocortisone, clobetasol propionate, betamethasone dipropionate, halobetasol propionate, diflorasone diacetate, fluocinonide, halcinonide, amcinonide, desoximetasone, triamcinolone acetonide, mometasone furoate, fluticasone propionate, betamethasone dipropionate, halometasone, fluocinolone acetonide, hydrocortisone valerate, hydrocortisone butyrate, flurandrenolide, triamcinolone acetonide, mometasone furoate, fluticasone propionate, desonide, fluocinolone acetonide, hydrocortisone valerate, alclometasone dipropionate, triamcinolone acetonide, fluocinolone acetonide, desonide, or a combination thereof.

In some embodiments of any of the methods described herein, the atopic dermatitis therapy comprises diphenhydramine, tacrolimus, pimecrolimus, cyclosporine A, methotrexate, interferon gamma, mycophenolate mofetil, azathioprine, crisaborole, dupilumab, or a combination thereof.

Also provided herein are methods for treating a subject in need thereof that include administering to the subject a composition comprising an effective amount of a bacterial species selected from the group consisting of: Brevundimonas nasdae, Capnocytophaga sputigena, Moraxella sp., Neisseria elongata, Staphylococcus felis, Staphylococcus sciuri, and Streptococcus intermedius, and a combination thereof.

In some embodiments of any of the methods described herein, the subject has atopic dermatitis.

Also provided herein are methods for treating atopic dermatitis in a subject that include administering to the subject a composition comprising an effective amount of a bacterial species selected from the group consisting of: Brevundimonas nasdae, Capnocytophaga sputigena, Moraxella sp., Neisseria elongata, Staphylococcus felis, Staphylococcus sciuri, and Streptococcus intermedius, and a combination thereof.

Also provided herein are methods for treating atopic dermatitis in a subject that include (a) detecting a dysbiosis associated with atopic dermatitis in a sample from the subject; and (b) administering to the subject a composition comprising an effective amount of a bacterial species selected from the group consisting of: Brevundimonas nasdae, Capnocytophaga sputigena, Moraxella sp., Neisseria elongata, Staphylococcus felis, Staphylococcus sciuri, and Streptococcus intermedius, and a combination thereof.

In some embodiments of any of the methods described herein, detecting the dysbiosis associated with atopic dermatitis comprises determining bacterial gene expression in the sample from the subject.

In some embodiments of any of the methods described herein, detecting the dysbiosis associated with atopic dermatitis comprises determining bacterial composition in the sample from the subject.

In some embodiments of any of the methods described herein, detecting the dysbiosis associated with atopic dermatitis comprises determining that Corynebacterium genitalium, Corynebacterium matruchotii, Corynebacterium striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, Turicella otitidis, or a combination thereof, is increased in the sample from subject.

In some embodiments of any of the methods described herein, detecting the dysbiosis associated with atopic dermatitis comprises determining that Brevundimonas nasdae, Capnocytophaga sputigena, Moraxella sp., Neisseria elongata, Staphylococcus felis, Staphylococcus sciuri, Streptococcus intermedius, or a combination thereof, is decreased in the sample from subject.

In some embodiments of any of the methods described herein, Brevundimonas nasdae, Capnocytophaga sputigena, Moraxella sp., Neisseria elongata, Staphylococcus felis, Staphylococcus sciuri, Streptococcus intermedius, or a combination thereof, is decreased in the gastrointestinal tract of the subject.

In some embodiments of any of the methods described herein, the sample is a skin swab sample.

Also provided herein are methods for treating a subject in need thereof that include decreasing a population of an increased bacterial strain in the subject, wherein the increased bacterial strain is selected from the group consisting of: Corynebacterium genitalium, Corynebacterium matruchotii, Corynebacterium striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, Turicella otitidis, and a combination thereof.

In some embodiments of any of the methods described herein, the subject has atopic dermatitis.

In some embodiments of any of the methods described herein, decreasing the population of an increased bacterial strain comprises administering to the subject a bacteriophage.

In some embodiments of any of the methods described herein, decreasing the population of an increased bacterial strain comprises administering to the subject a composition comprising an effective amount of a bacterial species selected from the group consisting of: Brevundimonas nasdae, Capnocytophaga sputigena, Moraxella sp., Neisseria elongata, Staphylococcus felis, Staphylococcus sciuri, Streptococcus intermedius, and a combination thereof.

In some embodiments of any of the methods described herein, the bacterial species Corynebacterium genitalium comprises the bacterial strain Corynebacterium genitalium 418H.

In some embodiments of any of the methods described herein, the bacterial species Corynebacterium matruchotii comprises the bacterial strain Corynebacterium matruchotii ATCC 33806.

In some embodiments of any of the methods described herein, the bacterial species Corynebacterium striatum comprises the bacterial strain Corynebacterium striatum ATCC 6940.

In some embodiments of any of the methods described herein, the bacterial species Corynebacterium matruchotii comprises the bacterial strain Corynebacterium tuberculostearicum CIP 102857.

In some embodiments of any of the methods described herein, the bacterial species Finegoldia magna comprises the bacterial strain Finegoldia magna 312.

In some embodiments of any of the methods described herein, the bacterial species Gordonia terrae comprises the bacterial strain Gordonia terrae T5.

In some embodiments of any of the methods described herein, the bacterial species Micrococcus luteus comprises the bacterial strain Micrococcus luteus SK58.

In some embodiments of any of the methods described herein, the bacterial species Peptococcus saccharolyticus comprises the bacterial strain Peptococcus saccharolyticus S1.

In some embodiments of any of the methods described herein, the bacterial species Prevotella oris comprises the bacterial strain Prevotella oris F0302.

In some embodiments of any of the methods described herein, the bacterial species Staphylococcus aureus comprises the bacterial strain Staphylococcus aureus C-243, Staphylococcus aureus MRSA252, Staphylococcus aureus MSSA476, Staphylococcus aureus MU3, Staphylococcus aureus ACM 2434, or a combination thereof.

In some embodiments of any of the methods described herein, the bacterial species Staphylococcus caprae comprises the bacterial strain Staphylococcus caprae 143.22, Staphylococcus caprae M23864 W1, or a combination thereof.

In some embodiments of any of the methods described herein, the bacterial species Staphylococcus cohnii comprises the bacterial strain Staphylococcus cohnii CCS 85.

In some embodiments of any of the methods described herein, the bacterial species Staphylococcus devriesei comprises the bacterial strain Staphylococcus devriesei KS SP60.

In some embodiments of any of the methods described herein, the bacterial species Staphylococcus epidermidis comprises the bacterial strain Staphylococcus epidermidis WHO 12.

In some embodiments of any of the methods described herein, the bacterial species Staphylococcus hominis comprises the bacterial strain Staphylococcus hominis DM 122.

In some embodiments of any of the methods described herein, the bacterial species Streptococcus mitis comprises the bacterial strain Streptococcus mitis R360.

In some embodiments of any of the methods described herein, the bacterial species Turicella otitidis comprises the bacterial strain Turicella otitidis 234/92.

In some embodiments of any of the methods described herein, the bacterial species Brevundimonas nasdae comprises the bacterial strain Brevundimonas nasdae W1-2B.

In some embodiments of any of the methods described herein, the bacterial species Capnocytophaga sputigena comprises the bacterial strain Capnocytophaga sputigena 4.

In some embodiments of any of the methods described herein, the bacterial species Moraxella sp. comprises the bacterial strain Moraxella sp. LMG 5131.

In some embodiments of any of the methods described herein, the bacterial species Neisseria elongata comprises the bacterial strain Neisseria elongata ATCC 29315.

In some embodiments of any of the methods described herein, the bacterial species Staphylococcus felis comprises the bacterial strain Staphylococcus felis GD521.

In some embodiments of any of the methods described herein, the bacterial species Staphylococcus sciuri comprises the bacterial strain Staphylococcus sciuri SC116.

In some embodiments of any of the methods described herein, the bacterial species Streptococcus intermedius comprises the bacterial strain Streptococcus intermedius 393.

In some embodiments of any of the methods described herein, the Brevundimonas nasdae W1-2B has a 16S RNA gene that is at least 95% identical to SEQ ID NO:1.

In some embodiments of any of the methods described herein, the Capnocytophaga sputigena 4 has a 16S RNA gene that is at least 95% identical to SEQ ID NO:2.

In some embodiments of any of the methods described herein, the Capnocytophaga sputigena 4 has a 16S RNA gene that is at least 95% identical to SEQ ID NO:3.

In some embodiments of any of the methods described herein, the Capnocytophaga sputigena 4 has a 16S+RNA gene that is at least 95% identical to SEQ ID NO:4.

In some embodiments of any of the methods described herein, the Capnocytophaga sputigena 4 has a 16S RNA gene that is at least 95% identical to SEQ ID NO:5.

In some embodiments of any of the methods described herein, the Capnocytophaga sputigena 4 has a 16S RNA gene that is at least 95% identical to SEQ ID NO:6.

In some embodiments of any of the methods described herein, the Moraxella sp. LMG 5131 has a 16S RNA gene that is at least 95% identical to SEQ ID NO:7.

In some embodiments of any of the methods described herein, the Moraxella sp. LMG 5131 has a 16S RNA gene that is at least 95% identical to SEQ ID NO:8.

In some embodiments of any of the methods described herein, the Moraxella sp. LMG 5131 has a 16S RNA gene that is at least 95% identical to SEQ ID NO:9.

In some embodiments of any of the methods described herein, the Moraxella sp. LMG 5131 has a 16S RNA gene that is at least 95% identical to SEQ ID NO:10.

In some embodiments of any of the methods described herein, the Moraxella sp. LMG 5131 has a 16S RNA gene that is at least 95% identical to SEQ ID NO:11.

In some embodiments of any of the methods described herein, the Neisseria elongata ATCC 29315 has a 16S RNA gene that is at least 95% identical to SEQ ID NO:12.

In some embodiments of any of the methods described herein, the Neisseria elongata ATCC 29315 has a 16S RNA gene that is at least 95% identical to SEQ ID NO:13.

In some embodiments of any of the methods described herein, the Neisseria elongata ATCC 29315 has a 16S RNA gene that is at least 95% identical to SEQ ID NO:14.

In some embodiments of any of the methods described herein, the Neisseria elongata ATCC 29315 has a 16S RNA gene that is at least 95% identical to SEQ ID NO:15.

In some embodiments of any of the methods described herein, the Neisseria elongata ATCC 29315 has a 16S RNA gene that is at least 95% identical to SEQ ID NO:16.

In some embodiments of any of the methods described herein, the Neisseria elongata ATCC 29315 has a 16S RNA gene that is at least 95% identical to SEQ ID NO:17.

In some embodiments of any of the methods described herein, the Staphylococcus felis GD521 has a 16S RNA gene that is at least 95% identical to SEQ ID NO:18.

In some embodiments of any of the methods described herein, the Staphylococcus sciuri SC116 has a 16S RNA gene that is at least 95% identical to SEQ ID NO:19.

In some embodiments of any of the methods described herein, the Streptococcus intermedius 393 has a 16S RNA gene that is at least 95% identical to SEQ ID NO:20.

In some embodiments of any of the methods described herein, the Streptococcus intermedius 393 has a 16S RNA gene that is at least 95% identical to SEQ ID NO:21.

In some embodiments of any of the methods described herein, the Streptococcus intermedius 393 has a 16S RNA gene that is at least 95% identical to SEQ ID NO:22.

In some embodiments of any of the methods described herein, the Streptococcus intermedius 393 has a 16S RNA gene that is at least 95% identical to SEQ ID NO:23.

In some embodiments of any of the methods described herein, the Streptococcus intermedius 393 has a 16S RNA gene that is at least 95% identical to SEQ ID NO:24.

In some embodiments of any of the methods described herein, the Streptococcus intermedius 393 has a 16S RNA gene that is at least 95% identical to SEQ ID NO:25.

In some embodiments of any of the methods described herein, the Streptococcus intermedius 393 has a 16S RNA gene that is at least 95% identical to SEQ ID NO:26.

In some embodiments of any of the methods described herein, the Streptococcus intermedius 393 has a 16S RNA gene that is at least 95% identical to SEQ ID NO:27.

In some embodiments of any of the methods described herein, the Streptococcus intermedius 393 has a 16S RNA gene that is at least 95% identical to SEQ ID NO:28.

In some embodiments of any of the methods described herein, the Streptococcus intermedius 393 has a 16S RNA gene that is at least 95% identical to SEQ ID NO:29.

In some embodiments of any of the methods described herein, the Streptococcus intermedius 393 has a 16S RNA gene that is at least 95% identical to SEQ ID NO:30.

In some embodiments of any of the methods described herein, administering the composition improves epidermal barrier function of the subject.

In some embodiments of any of the methods described herein, the bacterial species in the composition is viable.

In some embodiments of any of the methods described herein, the bacterial strain is lyophilized.

In some embodiments of any of the methods described herein, the composition further comprises one or more cryopreservants.

In some embodiments of any of the methods described herein, the effective amount of the bacterial strain comprises at least about 1×10³ colony forming units (CFU) of the bacterial strain.

In some embodiments of any of the methods described herein, the effective amount of the bacterial strain comprises about 1×10⁴ to about 1×10¹⁵ CFU of the bacterial strain.

In some embodiments of any of the methods described herein, the effective amount of the bacterial strain comprises about 1×10⁶ to about 1×10¹⁰ CFU of the bacterial strain.

In some embodiments of any of the methods described herein, the bacterial strain in the composition is non-viable.

In some embodiments of any of the methods described herein, the non-viable bacterial strain is heat-killed, irradiated, or lysed.

In some embodiments of any of the methods described herein, the method comprises administering the composition to the subject once, twice, or three times per day.

In some embodiments of any of the methods described herein, the composition is formulated for topical administration.

In some embodiments of any of the methods described herein, the composition is formulated as a paste, gel, cream, spray, mousse, emollient, ointment, foam, or suspension.

In some embodiments of any of the methods described herein, the composition is formulated for oral administration.

In some embodiments of any of the methods described herein, the composition is formulated for rectal administration.

In some embodiments of any of the methods described herein, the composition is formulated as a tablet, a capsule, a powder, or a liquid.

In some embodiments of any of the methods described herein, the composition is formulated as a tablet.

In some embodiments of any of the methods described herein, the tablet is coated.

In some embodiments of any of the methods described herein, the coating comprises an enteric coating.

In some embodiments of any of the methods described herein, the method further comprises administering another atopic dermatitis therapy and/or adjunct therapy to the subject.

In some embodiments of any of the methods described herein, the composition comprising the bacterial strain and the atopic dermatitis therapy and/or adjunct therapy are administered simultaneously.

In some embodiments of any of the methods described herein, the composition comprising the bacterial strain and the atopic dermatitis therapy and/or adjunct therapy are administered sequentially.

In some embodiments of any of the methods described herein, the composition comprising the bacterial strain further comprises the atopic dermatitis therapy and/or adjunct therapy.

In some embodiments of any of the methods described herein, the atopic dermatitis therapy and/or adjunct therapy comprises a probiotic.

In some embodiments of any of the methods described herein, the atopic dermatitis therapy and/or adjunct therapy comprises vitamin D, an antihistamine, a corticosteroid, a calcineurin inhibitor, an immunosuppressant, a phosphodiesterase-4 inhibitor, an anti-interleukin-4 receptor (IL-4Rα) antibody, an antibiotic, phototherapy, or a combination thereof.

In some embodiments of any of the methods described herein, the corticosteroid comprises hydrocortisone, clobetasol propionate, betamethasone dipropionate, halobetasol propionate, diflorasone diacetate, fluocinonide, halcinonide, amcinonide, desoximetasone, triamcinolone acetonide, mometasone furoate, fluticasone propionate, betamethasone dipropionate, halometasone, fluocinolone acetonide, hydrocortisone valerate, hydrocortisone butyrate, flurandrenolide, triamcinolone acetonide, mometasone furoate, fluticasone propionate, desonide, fluocinolone acetonide, hydrocortisone valerate, alclometasone dipropionate, triamcinolone acetonide, fluocinolone acetonide, desonide, or a combination thereof.

In some embodiments of any of the methods described herein, the atopic dermatitis therapy and/or adjunct therapy comprises diphenhydramine, tacrolimus, pimecrolimus, cyclosporine A, methotrexate, interferon gamma, mycophenolate mofetil, azathioprine, crisaborole, dupilumab, or a combination thereof.

In some embodiments of any of the methods described herein, the subject is a human.

Also provided herein are methods for treating a subject in need thereof that include administering to the subject a composition comprising an effective amount of a bacterial strain selected from the group consisting of: Brevundimonas nasdae W1-2B, Capnocytophaga sputigena 4, Moraxella sp. LMG 5131, Neisseria elongata ATCC 29315, Staphylococcus felis GD521, Staphylococcus sciuri SC116, Streptococcus intermedius 393, and a combination thereof.

In some embodiments of any of the methods described herein, the subject has atopic dermatitis.

Also provided herein are methods for treating atopic dermatitis in a subject that include administering to the subject a composition comprising an effective amount of a bacterial strain selected from the group consisting of: Brevundimonas nasdae W1-2B, Capnocytophaga sputigena 4, Moraxella sp. LMG 5131, Neisseria elongata ATCC 29315, Staphylococcus felis GD521, Staphylococcus sciuri SC116, Streptococcus intermedius 393, and a combination thereof.

Also provided herein are methods for treating atopic dermatitis in a subject that include (a) detecting a dysbiosis associated with atopic dermatitis in a sample from the subject; and (b) administering to the subject a composition comprising an effective amount of a bacterial strain selected from the group consisting of: Brevundimonas nasdae W1-2B, Capnocytophaga sputigena 4, Moraxella sp. LMG 5131, Neisseria elongata ATCC 29315, Staphylococcus felis GD521, Staphylococcus sciuri SC116, Streptococcus intermedius 393, and a combination thereof.

In some embodiments of any of the methods described herein, the sample is a skin swab sample.

In some embodiments of any of the methods described herein, detecting the dysbiosis associated with atopic dermatitis comprises determining bacterial gene expression in the sample from the subject.

In some embodiments of any of the methods described herein, detecting the dysbiosis associated with atopic dermatitis comprises determining bacterial composition in the sample from the subject.

In some embodiments of any of the methods described herein, detecting the dysbiosis associated with atopic dermatitis comprises determining that Corynebacterium genitalium 418H, Corynebacterium matruchotii ATCC 33806, Corynebacterium striatum ATCC 6940, Corynebacterium tuberculostearicum CIP 102857, Finegoldia magna 312, Gordonia terrae T5, Micrococcus luteus SK58, Peptococcus saccharolyticus S1, Prevotella oris F0302, Staphylococcus aureus C-243, Staphylococcus aureus MRSA252, Staphylococcus aureus MSSA476, Staphylococcus aureus MU3, Staphylococcus aureus ACM 2434, Staphylococcus caprae 143.22, Staphylococcus caprae M23864 W1, Staphylococcus cohnii CCS 85, Staphylococcus devriesei KS SP60, Staphylococcus epidermidis WHO 12, Staphylococcus hominis DM 122, Streptococcus mitis R 360, Turicella otitidis 234/92, or a combination thereof, is increased in the sample from subject.

In some embodiments of any of the methods described herein, detecting the dysbiosis associated with atopic dermatitis comprises determining that Brevundimonas nasdae W1-2B, Capnocytophaga sputigena 4, Moraxella sp. LMG 5131, Neisseria elongata ATCC 29315, Staphylococcus felis GD521, Staphylococcus sciuri SC116, Streptococcus intermedius 393, or a combination thereof, is decreased in the sample from subject.

In some embodiments of any of the methods described herein, Brevundimonas nasdae W1-2B, Capnocytophaga sputigena 4, Moraxella sp. LMG 5131, Neisseria elongata ATCC 29315, Staphylococcus felis GD521, Staphylococcus sciuri SC116, Streptococcus intermedius 393, or a combination thereof, is decreased in the gastrointestinal tract of the subject.

Also provided herein are methods for treating a subject in need thereof that include decreasing a population of an increased bacterial strain in the subject, wherein the increased bacterial strain is selected from the group consisting of: Corynebacterium genitalium 418H, Corynebacterium matruchotii ATCC 33806, Corynebacterium striatum ATCC 6940, Corynebacterium tuberculostearicum CIP 102857, Finegoldia magna 312, Gordonia terrae T5, Micrococcus luteus SK58, Peptococcus saccharolyticus S1, Prevotella oris F0302, Staphylococcus aureus C-243, Staphylococcus aureus MRSA252, Staphylococcus aureus MSSA476, Staphylococcus aureus MU3, Staphylococcus aureus ACM 2434, Staphylococcus caprae 143.22, Staphylococcus caprae M23864 W1, Staphylococcus cohnii CCS 85, Staphylococcus devriesei KS SP60, Staphylococcus epidermidis WHO 12, Staphylococcus hominis DM 122, Streptococcus mitis R 360, Turicella otitidis 234/92, and a combination thereof.

In some embodiments of any of the methods described herein, the subject has atopic dermatitis.

In some embodiments of any of the methods described herein, decreasing the population of an increased bacterial strain comprises administering to the subject a bacteriophage.

In some embodiments of any of the methods described herein, decreasing the population of an increased bacterial strain comprises administering to the subject a composition comprising an effective amount of a bacterial strain selected from the group consisting of: Brevundimonas nasdae W1-2B, Capnocytophaga sputigena 4, Moraxella sp. LMG 5131, Neisseria elongata ATCC 29315, Staphylococcus felis GD521, Staphylococcus sciuri SC116, Streptococcus intermedius 393, and a combination thereof.

In some embodiments of any of the methods described herein, the bacterial strain comprises Brevundimonas nasdae W1-2B.

In some embodiments of any of the methods described herein, the bacterial strain comprises Capnocytophaga sputigena 4.

In some embodiments of any of the methods described herein, the bacterial strain comprises Moraxella sp. LMG 5131.

In some embodiments of any of the methods described herein, the bacterial strain comprises Neisseria elongata ATCC 29315.

In some embodiments of any of the methods described herein, the bacterial strain comprises Staphylococcus felis GD521.

In some embodiments of any of the methods described herein, the bacterial strain comprises Staphylococcus sciuri SC116.

In some embodiments of any of the methods described herein, the bacterial strain comprises Streptococcus intermedius 393.

In some embodiments of any of the methods described herein, the bacterial strain improves epidermal barrier function of the subject.

In some embodiments of any of the methods described herein, the Brevundimonas nasdae W1-2B has a 16S RNA gene that is at least 95% identical to SEQ ID NO:1.

In some embodiments of any of the methods described herein, the Capnocytophaga sputigena 4 has a 16S RNA gene that is at least 95% identical to SEQ ID NO:2.

In some embodiments of any of the methods described herein, the Capnocytophaga sputigena 4 has a 16S RNA gene that is at least 95% identical to SEQ ID NO:3.

In some embodiments of any of the methods described herein, the Capnocytophaga sputigena 4 has a 16S RNA gene that is at least 95% identical to SEQ ID NO:4.

In some embodiments of any of the methods described herein, the Capnocytophaga sputigena 4 has a 16S RNA gene that is at least 95% identical to SEQ ID NO:5.

In some embodiments of any of the methods described herein, the Capnocytophaga sputigena 4 has a 16S RNA gene that is at least 95% identical to SEQ ID NO:6.

In some embodiments of any of the methods described herein, the Moraxella sp. LMG 5131 has a 16S RNA gene that is at least 95% identical to SEQ ID NO:7.

In some embodiments of any of the methods described herein, the Moraxella sp. LMG 5131 has a 16S RNA gene that is at least 95% identical to SEQ ID NO:8.

In some embodiments of any of the methods described herein, the Moraxella sp. LMG 5131 has a 16S RNA gene that is at least 95% identical to SEQ ID NO:9.

In some embodiments of any of the methods described herein, the Moraxella sp. LMG 5131 has a 16S RNA gene that is at least 95% identical to SEQ ID NO:10.

In some embodiments of any of the methods described herein, the Moraxella sp. LMG 5131 has a 16S RNA gene that is at least 95% identical to SEQ ID NO:11.

In some embodiments of any of the methods described herein, the Neisseria elongata ATCC 29315 has a 16S RNA gene that is at least 95% identical to SEQ ID NO:12.

In some embodiments of any of the methods described herein, the Neisseria elongata ATCC 29315 has a 16S RNA gene that is at least 95% identical to SEQ ID NO:13.

In some embodiments of any of the methods described herein, the Neisseria elongata ATCC 29315 has a 16S RNA gene that is at least 95% identical to SEQ ID NO:14.

In some embodiments of any of the methods described herein, the Neisseria elongata ATCC 29315 has a 16S RNA gene that is at least 95% identical to SEQ ID NO:15.

In some embodiments of any of the methods described herein, the Neisseria elongata ATCC 29315 has a 16S RNA gene that is at least 95% identical to SEQ ID NO:16.

In some embodiments of any of the methods described herein, the Neisseria elongata ATCC 29315 has a 16S RNA gene that is at least 95% identical to SEQ ID NO:17.

In some embodiments of any of the methods described herein, the Staphylococcus felis GD521 has a 16S RNA gene that is at least 95% identical to SEQ ID NO:18.

In some embodiments of any of the methods described herein, the Staphylococcus sciuri SC116 has a 16S RNA gene that is at least 95% identical to SEQ ID NO:19.

In some embodiments of any of the methods described herein, the Streptococcus intermedius 393 has a 16S RNA gene that is at least 95% identical to SEQ ID NO:20.

In some embodiments of any of the methods described herein, the Streptococcus intermedius 393 has a 16S RNA gene that is at least 95% identical to SEQ ID NO:21.

In some embodiments of any of the methods described herein, the Streptococcus intermedius 393 has a 16S RNA gene that is at least 95% identical to SEQ ID NO:22.

In some embodiments of any of the methods described herein, the Streptococcus intermedius 393 has a 16S RNA gene that is at least 95% identical to SEQ ID NO:23.

In some embodiments of any of the methods described herein, the Streptococcus intermedius 393 has a 16S RNA gene that is at least 95% identical to SEQ ID NO:24.

In some embodiments of any of the methods described herein, the Streptococcus intermedius 393 has a 16S RNA gene that is at least 95% identical to SEQ ID NO:25.

In some embodiments of any of the methods described herein, the Streptococcus intermedius 393 has a 16S RNA gene that is at least 95% identical to SEQ ID NO:26.

In some embodiments of any of the methods described herein, the Streptococcus intermedius 393 has a 16S RNA gene that is at least 95% identical to SEQ ID NO:27.

In some embodiments of any of the methods described herein, the Streptococcus intermedius 393 has a 16S RNA gene that is at least 95% identical to SEQ ID NO:28.

In some embodiments of any of the methods described herein, the Streptococcus intermedius 393 has a 16S RNA gene that is at least 95% identical to SEQ ID NO:29.

In some embodiments of any of the methods described herein, the Streptococcus intermedius 393 has a 16S RNA gene that is at least 95% identical to SEQ ID NO:30.

In some embodiments of any of the methods described herein, the bacterial strain in the composition is viable.

In some embodiments of any of the methods described herein, the bacterial strain is lyophilized.

In some embodiments of any of the methods described herein, the composition further comprises one or more cryopreservants.

In some embodiments of any of the methods described herein, the effective amount of the bacterial strain comprises at least about 1×10³ colony forming units (CFU) of the bacterial strain.

In some embodiments of any of the methods described herein, the effective amount of the bacterial strain comprises about 1×10⁴ to about 1×10¹⁵ CFU of the bacterial strain.

In some embodiments of any of the methods described herein, the effective amount of the bacterial strain comprises about 1×10⁶ to about 1×10¹⁰ CFU of the bacterial strain.

In some embodiments of any of the methods described herein, the bacterial strain in the composition is non-viable.

In some embodiments of any of the methods described herein, the non-viable bacterial strain is heat-killed, irradiated, or lysed.

In some embodiments of any of the methods described herein, the method comprises administering the composition to the subject once, twice, or three times per day.

In some embodiments of any of the methods described herein, the composition is formulated for topical administration.

In some embodiments of any of the methods described herein, the composition is formulated as a paste, gel, cream, spray, mousse, emollient, ointment, foam, or suspension.

In some embodiments of any of the methods described herein, the composition is formulated for oral administration.

In some embodiments of any of the methods described herein, the composition is formulated for rectal administration.

In some embodiments of any of the methods described herein, the composition is formulated as a tablet, a capsule, a powder, or a liquid.

In some embodiments of any of the methods described herein, the composition is formulated as a tablet.

In some embodiments of any of the methods described herein, the tablet is coated.

In some embodiments of any of the methods described herein, the coating comprises an enteric coating.

In some embodiments of any of the methods described herein, the method further comprises administering another treatment for atopic dermatitis and/or adjunct therapy to the subject.

In some embodiments of any of the methods described herein, the composition comprising the bacterial strain treatment and the treatment for atopic dermatitis and/or adjunct therapy are administered simultaneously.

In some embodiments of any of the methods described herein, the composition comprising the bacterial strain treatment and the treatment for atopic dermatitis and/or adjunct therapy are administered sequentially.

In some embodiments of any of the methods described herein, the composition comprising the bacterial strain further comprises the treatment for atopic dermatitis and/or adjunct therapy.

In some embodiments of any of the methods described herein, the treatment for atopic dermatitis and/or adjunct therapy comprises a probiotic.

In some embodiments of any of the methods described herein, the treatment for atopic dermatitis and/or adjunct therapy comprises vitamin D, an antihistamine, a corticosteroid, a calcineurin inhibitor, an immunosuppressant, a phosphodiesterase-4 inhibitor, an anti-interleukin-4 receptor (IL-4Rα) antibody, an antibiotic, phototherapy, or a combination thereof.

In some embodiments of any of the methods described herein, the corticosteroid comprises hydrocortisone, clobetasol propionate, betamethasone dipropionate, halobetasol propionate, diflorasone diacetate, fluocinonide, halcinonide, amcinonide, desoximetasone, triamcinolone acetonide, mometasone furoate, fluticasone propionate, betamethasone dipropionate, halometasone, fluocinolone acetonide, hydrocortisone valerate, hydrocortisone butyrate, flurandrenolide, triamcinolone acetonide, mometasone furoate, fluticasone propionate, desonide, fluocinolone acetonide, hydrocortisone valerate, alclometasone dipropionate, triamcinolone acetonide, fluocinolone acetonide, desonide, or a combination thereof.

In some embodiments of any of the methods described herein, the treatment for atopic dermatitis and/or adjunct therapy comprises diphenhydramine, tacrolimus, pimecrolimus, cyclosporine A, methotrexate, interferon gamma, mycophenolate mofetil, azathioprine, crisaborole, dupilumab, or a combination thereof.

In some embodiments of any of the methods described herein, the subject is a human.

Also provided herein are methods of characterizing a biopsy sample obtained from a subject as comprising a lesion or healthy tissue that include: characterizing a biopsy sample that has: (i) an increased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) bacterial species selected from: Corynebacterium genitalium, Corynebacterium matruchotii, Corynebacterium.striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; and/or (ii) a decreased level of one or more (e.g., two or more, three or more, four or more, five or more, six or more, or seven) bacterial species selected from: Brevundimonas nasdae, Capnocytophaga sputigena, Moraxella sp., Neisseria elongata, Staphylococcus felis, Staphylococcus sciuri, and Streptococcus intermedius; as comprising a lesion; or (b) characterizing a biopsy sample that does not have: (i) an increased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) bacterial species selected from: Corynebacterium genitalium, Corynebacterium matruchotii, Corynebacterium.striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; or (ii) a decreased level of one or more (e.g., two or more, three or more, four or more, five or more, six or more, or seven) bacterial species selected from: Brevundimonas nasdae, Capnocytophaga sputigena, Moraxella sp., Neisseria elongata, Staphylococcus felis, Staphylococcus sciuri, and Streptococcus intermedius; as comprising healthy tissue (not comprising a lesion).

As used herein, the phrase an “effective amount” of a bacterial strain can refer to an amount of the bacterial strain sufficient enough to reduce or eliminate one or more symptoms of the disorder or in some cases, to effect a cure upon administration. Effective amounts of a bacterial strain will vary with the bacterial strain chosen, the particular condition or conditions being treated, the severity of the condition, the duration of the treatment, the specific components of the composition being used, and like factors. An “effective amount” can also refer to an amount of a combination of two or more bacterial strains or a combination of a bacterial strain and another treatment and/or other adjunct therapy sufficient to reduce or eliminate one or more symptoms of the disorder or in some cases, to effect a cure upon administration. For example, an “effective amount” can refer to an amount of a combination of bacterial strains or a combination of a bacterial strain and another treatment (e.g., a therapeutic agent) when an additive or synergistic effect is observed with the combination compared to administration of the bacterial strain(s) and/or treatment(s) of atopic dermatitis alone.

As used herein, “subject” or “patient” refers to any subject, particularly a mammalian subject such as a human, for whom diagnosis, prognosis, or therapy is desired.

As used herein, “treatment” or “treating” of a disease, disorder, or condition encompasses alleviation of at least one symptom thereof, a reduction in the severity thereof, or the delay or inhibition of the progression thereof. Treatment need not mean that the disease, disorder, or condition is totally cured. A useful composition herein needs only to reduce the severity of a disease, disorder, or condition, reduce the severity of one or more symptoms associated therewith, or improve a patient or subject's quality of life.

The term “preventing” as used herein means the prevention of the onset, recurrence, or spread, in whole or in part, of the disease or condition as described herein, or a symptom thereof.

The term “administration” or “administering” refers to a method of giving an amount of bacterial strain, or a composition thereof, or a treatment of atopic dermatitis and/or other adjunct therapy to a subject. The method of administration can vary depending on various factors, e.g., the components of the composition, the site of the disease, and the severity of the disease.

“Microbiome” refers to the collection of microorganisms and viruses and/or their genes from a given environment. For example, “microbiome” can refer to the collection of the microorganisms and viruses and/or their genes from the gastrointestinal tract of humans. “Microbiota” refers to the microorganisms in a specific environment.

“Dysbiosis” refers to a state of the microbiota or microbiome of the gut or other body area (e.g., mucosal or skin surfaces or any other microbiota niche) of a subject (i.e., the host) in which the diversity and/or function of the ecological network is disrupted, e.g., as compared to the state of the microbiota or microbiome of the gut or other body area in a control population. A control population can include individuals that meet one or more qualifications such as individuals that have not been diagnosed with a disease (e.g., the same disease as the subject); individuals that do not have a known genetic predisposition to a disease (e.g., the same disease as the subject); or individuals that do not have a known environmental predisposition to a disease (e.g., the same disease as the subject); or individuals that do not have a known predisposition that would prevent treatment of and/or recovery from a disease (e.g., the same disease as the subject). In some embodiments, the individuals in the control population meet one of the above control population qualifications. In some embodiments, the individuals in the control population meet two of the above control population qualifications. In some embodiments, the individuals in the control population meet three of the above control population qualifications. In some embodiments, the individuals in the control population meet four of the above control population qualifications. In some embodiments, the control population is homogenous with respect to at least one of the qualifications. Any disruption in the microbiota or microbiome of a subject (i.e., host) compared to the microbiota or microbiome of a control population can be considered a dysbiosis, even if such dysbiosis does not result in a detectable decrease in health of the subject. Dysbiosis in a subject may be unhealthy for the subject (e.g., result in a diseased state in the subject), it may be unhealthy for the subject under only certain conditions (e.g., result in diseased state under only certain conditions), or it may prevent the subject from becoming healthier (e.g., may prevent a subject from responding to treatment or recovering from a disease or disorder). Dysbiosis may be due to a decrease in diversity of the microbiota population composition (e.g., a depletion of one or more bacterial strains, an overgrowth of one or more bacterial strains, or a combination thereof), the overgrowth of one or more population of pathogens (e.g., a population of pathogenic bacteria) or pathobionts, the presence of and/or overgrowth of a symbiotic organism able to cause disease only when certain genetic and/or environmental conditions are present in a subject, or a shift to an ecological network that no longer provides a beneficial function to the host and therefore no longer promotes health.

As used herein the terms “microorganism” or “microbe” should be taken broadly. These terms are used interchangeably and include, but are not limited to, the two prokaryotic domains, Bacteria and Archaea, as well as eukaryotic fungi and protists. In some embodiments, the disclosure refers to a “bacterium” or a “microbe.” This characterization can refer to not only the identified taxonomic bacterial genera of the microbe, but also the identified taxonomic species, as well as the bacterial strains. A “strain” can include descendants of a single isolation in pure culture that is usually made up of a succession of cultures ultimately derived from an initial single colony. In some embodiments, a strain includes an isolate or a group of isolates that can be distinguished from other isolates of the same genus and species by phenotypic characteristics, genotypic characteristics, or both.

The term “relative abundance” as used herein, is the number or percentage of a microbe present in the gastrointestinal tract or any other microbiota niche of a subject, such as the ocular, placental, lung, cutaneous, urogenital, or oral microbiota niches, relative to the number or percentage of total microbes present in the gastrointestinal tract or the other microbiota niche of the subject. The relative abundance may also be determined for particular types of microbes such as bacteria, fungi, viruses, and/or protozoa, relative to the total number or percentage of bacteria, fungi, viruses, and/or protozoa present. Relative abundance can be determined by a number of methods readily known to the ordinarily skilled artisan, including, but not limited to, array or microarray hybridization, sequencing, quantitative PCR, and culturing and performance of colony forming unit (cfu, CFU) assays or plaque forming unit (pfu, PFU) assays performed on a sample from the gastrointestinal tract or other microbiota niche.

As used herein, terms such as “isolate” and “isolated” in reference to a microbe, are intended to mean that a microbe has been separated from at least one of the materials with which it is associated in a particular environment (for example gastrointestinal fluid, gastrointestinal tissue, human digestive fluid, human digestive tissue, etc.). Accordingly, an “isolated microbe” does not exist in its naturally occurring environment. In some embodiments, an isolated microbe, e.g., a bacterial strain, may exist as, for example, a biologically pure culture, or as spores (or other forms of the bacterial strain) in association with a pharmaceutically acceptable excipient suitable for human administration. In some embodiments, more than one microbe can be isolated. For example, “isolated microbes” can refer to a mixture of two or more microbes that have been separated from at least one of the materials with which they are associated in a particular environment.

In some embodiments, the isolated microbes exist as isolated and biologically pure cultures. As used herein, the term “biologically pure” refers to a composition comprising a species or strains of a microbe, wherein the composition is substantially free from the material from which the microbe was isolated or produced and from other microbes (e.g., other species or strains and other microbes of a different taxonomic classification). In some embodiments, “biologically pure” can refer to a composition that comprises a strain of a bacterial strain that is substantially free from the material from which the bacterial strain was isolated or produced and from other microbes, e.g., other strains of the same bacterial strain, other species of the same bacteria, and other bacteria and/or microbes of a different taxonomic classification). It will be appreciated by one of skill in the art, that an isolated and biologically pure culture of a particular microbe, denotes that said culture is substantially free (within scientific reason) of other living organisms and contains only the individual microbe in question. As used herein, “substantially free” means that a composition comprising a species or strain of a microbe is at least about 80%, about 85%, about 90%, about 91%, about 92%, about 93%, about 94%, about 95%, about 96%, about 97%, about 98%, about 99%, or more than about 99% free of the material from which the microbe was isolated or produced and from other microbes. In some embodiments, a biologically pure composition contains no other bacterial strain in quantities that can be detected by typical bacteriological techniques.

As used herein, the term “mutation” includes a natural or induced mutation comprising at least a single base or amino acid alteration in a DNA or protein sequence. For example, a mutation can include a base substitution, a deletion, an insertion, a transversion, or any other modification known to those skilled in the art, including a genetic modification introduced into a parent nucleotide or an amino acid sequence.

As used herein, “probiotic” refers to a substantially pure microbe (i.e., a single isolate) or a mixture of microbes, and may also include any additional components that can be administered to a subject (e.g., a human), for restoring or altering the microbiota or microbiome in the subject. In some embodiments, a probiotic or microbial inoculant composition can be administered with an agent to allow the microbe(s) to survive the environment of the gastrointestinal tract, i.e., to resist low pH and/or to grow in the gastrointestinal environment. In some embodiments, a composition as described herein includes a probiotic.

As used herein, “prebiotic” refers to an agent that increases the number and/or activity of one or more microbes. Such microbes can include microbes for restoring or altering the microbiota or microbiome of a subject. Non-limiting examples of a prebiotic include a fructooligosaccharide (e.g., oligofructose, inulin, or an inulin-type fructan), a galactooligosaccharide, an amino acid, an alcohol. See, for example, Ramirez-Farias et al. (2008. Br. J Nutr. 4:1-10) and Pool-Zobel and Sauer (2007. J Nutr. 137:2580-2584).

As used herein, a “live biotherapeutic product” or “LBP” refers to a biological product that: 1) contains live organisms, such as bacteria, and 2) is applicable to the prevention, treatment, and/or cure of a disease or condition of a subject.

A “combination” of two or more bacteria, e.g., bacterial strains, can refer to the physical co-existence of the bacteria, either in the same material or product. In some embodiments, a combination of two or more bacteria can include the temporal co-administration or co-localization of the two or more bacteria.

The terms “percent identity” or “identity” in the context of two or more nucleic acids or polypeptides, refers to the measurement of the similarity between the two or more sequences. The percent identity can be measured by any method known to one of skill in the art including using a sequence comparison software, an algorithm, and by visual inspection.

In general, the percent identity for two or more sequences (e.g., a nucleic acid or amino acid sequence), also referred to as the “percent sequence identity”, is calculated by determining the number of matched positions in the aligned nucleic acid or amino acid sequences, dividing the number of matched positions by the total number of aligned nucleotides or amino acids, respectively, and multiplying by 100. A matched position refers to a position in which identical nucleotides or amino acids occur at the same position in the aligned sequences. As an example, the total number of aligned nucleotides can refer to the minimum number of the 16S rRNA gene nucleotides that are necessary to align the second sequence, and does not include alignment (e.g., forced alignment) with non-16S rRNA gene sequences. The total number of aligned nucleotides may correspond to the entire 16S rRNA gene sequence or may correspond to fragments of the full-length 16S rRNA gene sequence.

Sequences can be aligned using an algorithm, for example, the algorithm as described by Altschul et al. (Nucleic Acids Res, 25:3389-3402, 1997) and incorporated into BLAST (basic local alignment search tool) programs, which are available at ncbi.nlm.nih.gov. BLAST searches or alignments can be performed to determine percent sequence identity between a 16S rRNA gene nucleic acid and any other sequence or portion thereof using the Altschul et al. algorithm. BLASTN can be used to align and compare the identity between nucleic acid sequences, while BLASTP can be used to align and compare the identity between amino acid sequences. When utilizing a BLAST program to calculate the percent identity between a 16S rRNA gene sequence and another sequence, the default parameters of the program are used.

Generally, a bacterial strain genomic sequence will contain multiple copies of 16S rRNA sequences. The 16S rRNA sequences can be used for making distinctions between species and strains. For example, if one or more of the 16S rRNA sequences shares less than 97% sequence identity from a reference sequence, then the two organisms from which the sequences were obtained can be of different species or strains.

The term “combination therapy” as used herein refers to a dosing regimen of one or more bacterial strains and one or more other treatments of atopic dermatitis and/or other adjunct therapies during a period of time, wherein the bacterial strain and other treatment (e.g., a therapeutic agent) are administered together or separately in a manner prescribed by a medical care taker or according to a regulatory agency. As can be appreciated in the art, a combination therapy can be administered to a patient for a period of time. In some embodiments, the period of time occurs following the administration of one or more of: a different bacterial strain, a different treatment/therapeutic agent, and a different combination of treatments/therapeutic agents to the subject. In some embodiments, the period of time occurs before the administration of one or more of: a different bacterial strain, a different treatment/agent, and a different combination of treatments/therapeutic agents to the subject.

The term “fixed combination” means that one or more bacterial strains as described herein, or a composition thereof, and at least one other treatment and/or adjunct therapy (e.g., a prebiotic, a probiotic, an immunosuppressant, an anti-inflammatory agent, an antibiotic, an anti-diarrheal agent, a pain reliever, an iron supplement, or a combination thereof), are both administered to a subject simultaneously in the form of a single composition or dosage.

The term “non-fixed combination” means that one or more bacterial strains as described herein, or a composition thereof, and at least one other treatment or adjunct therapy (e.g., a prebiotic, a probiotic, an immunosuppressant, an anti-inflammatory agent, an antibiotic, an anti-diarrheal agent, a pain reliever, an iron supplement, or a combination thereof) are formulated as separate compositions or dosages such that they may be administered to a subject simultaneously or sequentially with variable intervening time limits. These also apply to cocktail therapies, e.g., the administration of three or more therapeutic agents.

Reference to the term “about” has its usual meaning in the context of compositions to allow for reasonable variations in amounts that can achieve the same effect and also refers herein to a value of plus or minus 10% of the provided value. For example, “about 20” means or includes amounts from 18 to and including 22.

Unless otherwise required by context, singular terms shall include pluralities and plural terms shall include the singular. As used herein, the singular form “a,” “an,” and “the” include plural references unless indicated otherwise. For example, “an” excipient includes one or more excipients. It is understood that aspects and variations of the invention described herein include “consisting of” and/or “consisting essentially of” aspects and variations.

The details of one or more embodiments of the invention are set forth in the accompanying drawings and the description below. Other features, objects, and advantages of the invention will be apparent from the description and drawings, and from the claims.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### DESCRIPTION OF DRAWINGS

FIG. 1 is an exemplary schematic of the analysis.

FIG. 2 has plots showing the tallies of strains detected (after prevalence filtering). The top panel tallies the strains detected in increasing number of datasets. The middle panel shows the number of strains that were significantly differentially abundant in 1 or more datasets. The bottom panel shows the number of strains that exhibit concordance in direction of the log 2 fold change, in increasing proportions of datasets in which they were detected. Only strains detected in at least two datasets are included.

FIG. 3 is a plot showing that significant findings from isolated dataset analysis rarely exhibits concordance in the direction of shifts across multiple datasets. Each row represents a strain identified as significantly differentially abundant by at least one isolated analysis. Asterisk denotes dataset(s) where significant changes of a strain was observed. Cell color indicates direction of the log 2 fold change: reduced (decreased) (green) or enriched (increased) (pink) in dysbiosis and not detected (white). Cells are shaded dark if the direction of log 2 fold change is concordant across all datasets a strain was detected in, i.e., the strain is associated with homeostasis or dysbiosis only. Two contrasts were analyzed from each of the two cohorts of patients. Results from analysis of cohort 1 and cohort 2 are represented in first and second columns, and third and fourth columns, respectively. See FIG. 8 for details on contrasts analyzed within each cohort of subjects.

FIG. 4 is a plot showing the distribution of effect sizes per dataset. 16S-NGS refers to sequencing of the 16S rRNA gene via next generation sequencing (NGS).

FIG. 5 is a plot showing strains (dots) significantly differentially abundant in eubiotic or dysbiotic state by isolated-dataset analysis (grey squares) or MTMA (grey spheres) in atopic dermatitis. Strains (dots) are sized by the number of datasets in which they were detected and colored as follows: significant by isolated analysis only (dark green); MTMA only (purple); or both (blue). Solid lines connect MTMA results to strains and dashed lines connect isolated analysis results to strains. Thick and thin lines indicate significant and non-significant findings, respectively. Red and green lines indicate enrichment (increase) and reduction (decrease) in dysbiotic state, respectively. Annotation for strains described here are provided in FIG. 9.

FIG. 6 is a forest plot demonstrating distribution of log 2 fold changes and 95% confidence intervals for strains that were identified as significantly differentially abundant by MTMA. Circles and triangles indicate log 2 fold change estimated by isolated analysis and MTMA, respectively. Error bars in the forest plots correspond to the 95% confidence interval. Green and blue indicate significant and nonsignificant findings, respectively, and grey indicates cases where an adjusted p-value could not be imputed by the statistical test.

FIG. 7 is a plot of MTMA-derived adjusted p-values and log 2 fold changes. Data points are shaded according to the proportion of datasets in which the strain was detected. Significantly dysbiosis-associated strains plot in the upper left quadrant, whereas homeostasis-associated strains plot in the upper right quadrant. FIG. 9 provides strain names for strain identifiers indicated in the plot.

FIG. 8 is a table showing details on contrasts analyzed within each cohort of subjects.

FIG. 9 is a table showing the association between the strain identifiers and strain names.

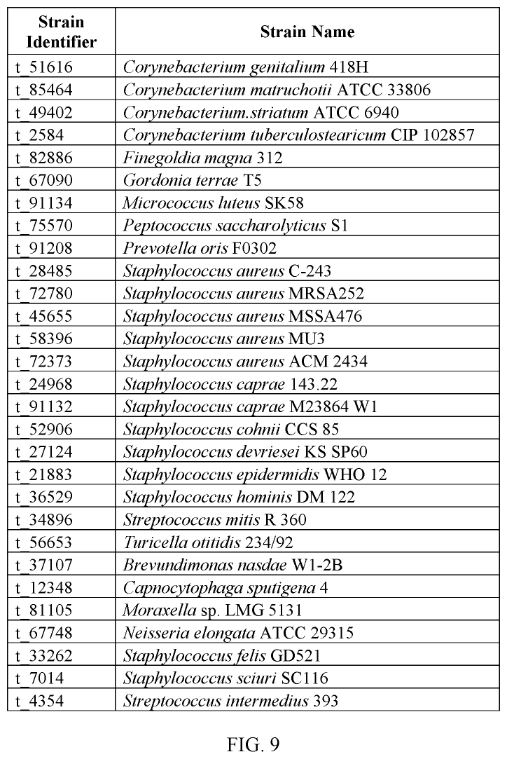

### DETAILED DESCRIPTION

This document provides compositions and methods for treating subjects in need thereof (e.g., subjects having atopic dermatitis) using one or more bacterial strains. Atopic dermatitis (AD) is a chronic, inflammatory skin disease (Rusu. Exp Ther Med. 2019 August; 18(2): 926-931). It can be characterized by pruritus, xerosis (dry skin), eczematous lesions, and lichenification. Chronic, localized, or even generalized pruritus are major characteristics of atopic dermatitis (Hong. Semin Cutan Med Surg. 2011 June; 30(2): 71-86.).

In some embodiments, methods for treating a subject in need thereof are provided herein. In some embodiments, one or more of Brevundimonas nasdae W1-2B, Capnocytophaga sputigena 4, Moraxella sp. LMG 5131, Neisseria elongata ATCC 29315, Staphylococcus felis GD521, Staphylococcus sciuri SC116, and Streptococcus intermedius 393 are reduced (decreased) in a sample (e.g., a fecal sample or a skin swab sample) from the subject in need thereof. For example, one or more of Brevundimonas nasdae W1-2B, Capnocytophaga sputigena 4, Moraxella sp. LMG 5131, Neisseria elongata ATCC 29315, Staphylococcus felis GD521, Staphylococcus sciuri SC116, and Streptococcus intermedius 393 are reduced in a sample (e.g., a fecal sample or a skin swab sample) from the subject in need thereof compared to a control sample. Determining that one or more of Brevundimonas nasdae W1-2B, Capnocytophaga sputigena 4, Moraxella sp. LMG 5131, Neisseria elongata ATCC 29315, Staphylococcus felis GD521, Staphylococcus sciuri SC116, and Streptococcus intermedius 393 is reduced (decreased) in the sample from the subject in need thereof can comprise sequencing one or more nucleic acids from the bacteria. In some embodiments, the subject in need thereof has been diagnosed with atopic dermatitis. The methods provided herein can include administering to the subject a composition that includes an effective amount of a bacterial strain. In some embodiments, the bacterial strain can be selected from the group consisting of: Brevundimonas nasdae W1-2B, Capnocytophaga sputigena 4, Moraxella sp. LMG 5131, Neisseria elongata ATCC 29315, Staphylococcus felis GD521, Staphylococcus sciuri SC116, Streptococcus intermedius 393, and a combination thereof (e.g., any two, any three, any four, any five, any six, or seven of the bacterial strains).

In some embodiments, the bacterial strain in the composition comprises Brevundimonas nasdae W1-2B. In some embodiments, Brevundimonas nasdae W1-2B included in a composition provided herein has a 16S RNA gene that is at least 90% identical to SEQ ID NO:1. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to SEQ ID NO:1

In some embodiments, the bacterial strain in the composition comprises Capnocytophaga sputigena 4. A complete genomic sequence for Capnocytophaga sputigena 4 is available in the GenBank database as, e.g., Accession No. GCF_000173675. In some embodiments, the Capnocytophaga sputigena 4 included in a composition provided herein can have a genomic sequence with at least about 95% sequence identity to the genomic sequence published as GCF_000173675. For example, Capnocytophaga sputigena 4 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000173675. In some embodiments, Capnocytophaga sputigena 4 included in a composition provided herein has a 16S RNA gene that is at least 90% identical to one or more (e.g., one, two, three, four, five, or all) of SEQ ID NO:2, SEQ ID NO:3, SEQ ID NO:4, SEQ ID NO:5, and SEQ ID NO:6. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, or all) of SEQ ID NO:2, SEQ ID NO:3, SEQ ID NO:4, SEQ ID NO:5, and SEQ ID NO:6.

In some embodiments, the bacterial strain in the composition comprises Moraxella sp. LMG 5131. A complete genomic sequence for Moraxella sp. LMG 5131 is available in the GenBank database as, e.g., Accession No. GCF_001553955. In some embodiments, the Moraxella sp. LMG 5131 included in a composition provided herein can have a genomic sequence with at least about 95% sequence identity to the genomic sequence published as GCF_001553955. For example, Moraxella sp. LMG 5131 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_001553955. In some embodiments, Moraxella sp. LMG 5131 included in a composition provided herein has a 16S RNA gene that is at least 90% identical to one or more (e.g., one, two, three, four, or all) of SEQ ID NO:7, SEQ ID NO:8, SEQ ID NO:9, SEQ ID NO:10, and SEQ ID NO:11. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, or all) of SEQ ID NO:7, SEQ ID NO:8, SEQ ID NO:9, SEQ ID NO:10, and SEQ ID NO:11.

In some embodiments, the bacterial strain in the composition comprises Neisseria elongata ATCC 29315. A complete genomic sequence for Neisseria elongata ATCC 29315 is available in the GenBank database as, e.g., Accession No. GCF_000176755 and GCF_000818035. In some embodiments, the Neisseria elongata ATCC 29315 included in a composition provided herein can have a genomic sequence with at least about 95% sequence identity to the genomic sequence published as GCF_000176755 and/or GCF_000818035. For example, Neisseria elongata ATCC 29315 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000176755 and/or GCF_000818035. In some embodiments, Neisseria elongata ATCC 29315 included in a composition provided herein has a 16S RNA gene that is at least 90% identical to one or more (e.g., one, two, three, four, five, or all) of SEQ ID NO:12, SEQ ID NO:13, SEQ ID NO:14, SEQ ID NO:15, SEQ ID NO:16, and SEQ ID NO:17. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, or all) of SEQ ID NO:12, SEQ ID NO:13, SEQ ID NO:14, SEQ ID NO:15, SEQ ID NO:16, and SEQ ID NO:17.

In some embodiments, the bacterial strain in the composition comprises Staphylococcus felis GD521. A complete genomic sequence for Staphylococcus felis GD521 is available in the GenBank database as, e.g., Accession No. GCF_000156495. In some embodiments, the Staphylococcus felis GD521 included in a composition provided herein can have a genomic sequence with at least about 95% sequence identity to the genomic sequence published as GCF_000156495. For example, Staphylococcus felis GD521 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000156495. In some embodiments, Staphylococcus felis GD521 included in a composition provided herein has a 16S RNA gene that is at least 90% identical to SEQ ID NO:18. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to SEQ ID NO:18.

In some embodiments, the bacterial strain in the composition comprises Staphylococcus sciuri SC116. A complete genomic sequence for Staphylococcus sciuri SC116 is available in the GenBank database as, e.g., Accession No. GCF_001046995. In some embodiments, the Staphylococcus sciuri SC116 included in a composition provided herein can have a genomic sequence with at least about 95% sequence identity to the genomic sequence published as GCF_001046995. For example, Staphylococcus sciuri SC116 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_001046995. In some embodiments, Staphylococcus sciuri SC116 included in a composition provided herein has a 16S RNA gene that is at least 90% identical to SEQ ID NO:19. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to SEQ ID NO:19.

In some embodiments, the bacterial strain in the composition comprises Streptococcus intermedius 393. A complete genomic sequence for Streptococcus intermedius 393 is available in the GenBank database as, e.g., Accession No. GCF_000474095, GCF_000413475, and GCF_000258445. In some embodiments, the Streptococcus intermedius 393 included in a composition provided herein can have a genomic sequence with at least about 95% sequence identity to the genomic sequence published as one or more of GCF_000474095, GCF_000413475, and GCF_000258445. For example, Streptococcus intermedius 393 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as one or more of GCF_000474095, GCF_000413475, and GCF_000258445. In some embodiments, Streptococcus intermedius 393 included in a composition provided herein has a 16S RNA gene that is at least 90% identical to one or more (e.g., one, two, three, four, five, six, seven, eight, nine, ten, or all) of SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, and SEQ ID NO:30. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, six, seven, eight, nine, ten, or all) of SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, and SEQ ID NO:30.

In some embodiments, the composition can include two or more bacterial strains selected from the group consisting of: Brevundimonas nasdae W1-2B, Capnocytophaga sputigena 4, Moraxella sp. LMG 5131, Neisseria elongata ATCC 29315, Staphylococcus felis GD521, Staphylococcus sciuri SC116, and Streptococcus intermedius 393. For example, the composition can include three or more, four or more, five or more, six or more, or all seven bacterial strains selected from the group consisting of: Brevundimonas nasdae W1-2B, Capnocytophaga sputigena 4, Moraxella sp. LMG 5131, Neisseria elongata ATCC 29315, Staphylococcus felis GD521, Staphylococcus sciuri SC116, and Streptococcus intermedius 393. Identifying characteristics of each strain are described above.

In some embodiments, a method can include detecting, in a sample from the subject, a dysbiosis associated with atopic dermatitis, e.g., before administering to the subject an effective amount of a bacterial strain or a composition containing the bacterial strain. The sample can be a skin swab sample and/or a fecal sample. See, e.g., Watanabe et al. J Allergy Clin. Immunol. 2003, 111(3):587-91.

In some embodiments, detecting the dysbiosis associated with atopic dermatitis can include determining bacterial gene expression in the sample from the subject. (e.g., fecal sample or a skin swab sample). For example, the bacterial gene expression can be determined in the sample from the subject e.g., before administering to the subject an effective amount of a bacterial strain or a composition containing the bacterial strain and/or after administering to the subject an effective amount of a bacterial strain or a composition containing the bacterial strain. Determining the bacterial gene expression can include performing, for example, RNAseq and/or RT-qPCR. In some embodiments, detecting the dysbiosis associated with atopic dermatitis comprises determining bacterial composition in the sample from the subject (e.g., a skin swab sample). For example, the bacterial composition can be determined in a sample from the subject, e.g., before administering to the subject an effective amount of a bacterial strain or a composition containing the bacterial strain and/or after administering to the subject an effective amount of a bacterial strain or a composition containing the bacterial strain. Determining the bacterial composition can include, for example, sequencing one or more nucleic acids from the bacteria. In some embodiments, bacteria can be identified by their 16S rRNA gene sequence.

In some embodiments, detecting the dysbiosis comprises determining that Brevundimonas nasdae W1-2B, Capnocytophaga sputigena 4, Moraxella sp. LMG 5131, Neisseria elongata ATCC 29315, Staphylococcus felis GD521, Staphylococcus sciuri SC116, Streptococcus intermedius 393, or a combination thereof, is reduced (decreased) in the sample from subject (e.g., reduced (decreased) on the skin of the subject). In some embodiments, detecting the dysbiosis comprises determining that Brevundimonas nasdae W1-2B, Capnocytophaga sputigena 4, Moraxella sp. LMG 5131, Neisseria elongata ATCC 29315, Staphylococcus felis GD521, Staphylococcus sciuri SC116, Streptococcus intermedius 393, or a combination thereof, is reduced (decreased) in the sample from subject (e.g., reduced (decreased) on the skin of the subject).

In some embodiments, detecting the dysbiosis associated with atopic dermatitis comprises determining that Corynebacterium genitalium 418H, Corynebacterium matruchotii ATCC 33806, Corynebacterium.striatum ATCC 6940, Corynebacterium tuberculostearicum CIP 102857, Finegoldia magna 312, Gordonia terrae T5, Micrococcus luteus SK58, Peptococcus saccharolyticus S1, Prevotella oris F0302, Staphylococcus aureus C-243, Staphylococcus aureus MRSA252, Staphylococcus aureus MSSA476, Staphylococcus aureus MU3, Staphylococcus aureus ACM 2434, Staphylococcus caprae 143.22, Staphylococcus caprae M23864 W1, Staphylococcus cohnii CCS 85, Staphylococcus devriesei KS SP60, Staphylococcus epidermidis WHO 12, Staphylococcus hominis DM 122, Streptococcus mitis R 360, Turicella otitidis 234/92, or a combination thereof, is enriched (increased) in the sample from subject.

In some embodiments, a method as provided herein can include decreasing a population of an enriched (increased) bacterial strain in a subject (e.g., a subject with atopic dermatitis). In some embodiments, detecting the decrease in the population of an enriched (increased) bacterial strain comprises determining the bacterial composition in a sample from the subject (e.g., a fecal sample or a skin swab sample). For example, the bacterial composition can be determined in a sample from the subject before administering to the subject an effective amount of a bacterial strain or a composition containing the bacterial strain and after administering to the subject an effective amount of a bacterial strain or a composition containing the bacterial strain. For example, the population of an enriched (increased) bacterial strain can be decreased by at least 1%, at least 2%, at least 3%, at least 4%, at least 5%, at least 6%, at least 7%, at least 10%, at least 20%, at least 25%, at least 30%, at least 35%, at least 40%, or at least 50%, e.g., in a sample from the subject after administration of a bacterial strain or a composition containing the bacterial strain to the subject compared to before administration to the subject of an effective amount of a bacterial strain or a composition containing the bacterial strain. Determining the bacterial composition can include, for example, sequencing one or more nucleic acids from the bacteria. In some embodiments, bacteria can be identified by their 16S rRNA gene sequence.

In some embodiments, the enriched (increased) bacterial strain can be selected from the group consisting of: Corynebacterium genitalium 418H, Corynebacterium matruchotii ATCC 33806, Corynebacterium.striatum ATCC 6940, Corynebacterium tuberculostearicum CIP 102857, Finegoldia magna 312, Gordonia terrae T5, Micrococcus luteus SK58, Peptococcus saccharolyticus S1, Prevotella oris F0302, Staphylococcus aureus C-243, Staphylococcus aureus MRSA252, Staphylococcus aureus MSSA476, Staphylococcus aureus MU3, Staphylococcus aureus ACM 2434, Staphylococcus caprae 143.22, Staphylococcus caprae M23864 W1, Staphylococcus cohnii CCS 85, Staphylococcus devriesei KS SP60, Staphylococcus epidermidis WHO 12, Staphylococcus hominis DM 122, Streptococcus mitis R 360, Turicella otitidis 234/92, and a combination thereof.

In some embodiments, Corynebacterium genitalium 418H has a 16S RNA gene that is at least 90% identical to SEQ ID NO:31. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to SEQ ID NO:31.

In some embodiments, Corynebacterium matruchotii ATCC 33806 has a 16S RNA gene that is at least 90% identical to one or more (e.g., one, two, or all) of SEQ ID NO:32, SEQ ID NO:33, and SEQ ID NO:34. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, or all) of SEQ ID NO:32, SEQ ID NO:33, and SEQ ID NO:34.

In some embodiments, Corynebacterium.striatum ATCC 6940 has a 16S RNA gene that is at least 90% identical to one or more (e.g., one, two, three, or all) of SEQ ID NO:35, SEQ ID NO:36, SEQ ID NO:37, and SEQ ID NO:38. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, or all) of SEQ ID NO:35, SEQ ID NO:36, SEQ ID NO:37, and SEQ ID NO:38.

In some embodiments, Corynebacterium tuberculostearicum CIP 102857 has a 16S RNA gene that is at least 90% identical to SEQ ID NO:39. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to SEQ ID NO:39.

In some embodiments, Finegoldia magna 312 has a 16S RNA gene that is at least 90% identical to one or more (e.g., one, two, three, or all) of SEQ ID NO:40, SEQ ID NO:41, SEQ ID NO:42, and SEQ ID NO:43. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, or all) of SEQ ID NO:40, SEQ ID NO:41, SEQ ID NO:42, and SEQ ID NO:43.

In some embodiments, Gordonia terrae T5 has a 16S RNA gene that is at least 90% identical to one or more (e.g., one, two, three, or all) SEQ ID NO:44, SEQ ID NO:45, SEQ ID NO:46, and SEQ ID NO:47. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, or all) SEQ ID NO:44, SEQ ID NO:45, SEQ ID NO:46, and SEQ ID NO:47.

In some embodiments, Micrococcus luteus SK58 has a 16S RNA gene that is at least 90% identical to one or more (e.g., one, two, three, or all) of SEQ ID NO:48, SEQ ID NO:49, SEQ ID NO:50, and SEQ ID NO:51. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, or all) of SEQ ID NO:48, SEQ ID NO:49, SEQ ID NO:50, and SEQ ID NO:51.

In some embodiments, Peptococcus saccharolyticus 51 has a 16S RNA gene that is at least 90% identical to one or both of SEQ ID NO:52 and SEQ ID NO:53. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or both of SEQ ID NO:52 and SEQ ID NO:53.

In some embodiments, Prevotella oris F0302 has a 16S RNA gene that is at least 90% identical to one or more (e.g., one, two, three, or all) of SEQ ID NO:54, SEQ ID NO:55, SEQ ID NO:56, and SEQ ID NO:57. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, or all) of SEQ ID NO:54, SEQ ID NO:55, SEQ ID NO:56, and SEQ ID NO:57.

In some embodiments, Staphylococcus aureus C-243 has a 16S RNA gene that is at least 90% identical to one or both of SEQ ID NO:77 and SEQ ID NO:78. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or both of SEQ ID NO:77 and SEQ ID NO:78.

In some embodiments, Staphylococcus aureus MRSA252 has a 16S RNA gene that is at least 90% identical to one or more (e.g., one, two, three, four, five, or all) SEQ ID NO:79, SEQ ID NO:80, SEQ ID NO:81, SEQ ID NO:82, SEQ ID NO:83, and SEQ ID NO:84. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, or all) SEQ ID NO:79, SEQ ID NO:80, SEQ ID NO:81, SEQ ID NO:82, SEQ ID NO:83, and SEQ ID NO:84.

In some embodiments, Staphylococcus aureus MSSA476 has a 16S RNA gene that is at least 90% identical to one or more (e.g., one, two, three, four, five, six, seven, eight, nine, ten, eleven, twelve, or all) of SEQ ID NO:85, SEQ ID NO:86, SEQ ID NO:87, SEQ ID NO:88, SEQ ID NO:89. SEQ ID NO:90, SEQ ID NO:91, SEQ ID NO:92, SEQ ID NO:93, SEQ ID NO:94, SEQ ID NO:95, SEQ ID NO:96, and SEQ ID NO:97. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, six, seven, eight, nine, ten, eleven, twelve, or all) of SEQ ID NO:85, SEQ ID NO:86, SEQ ID NO:87, SEQ ID NO:88, SEQ ID NO:89. SEQ ID NO:90, SEQ ID NO:91, SEQ ID NO:92, SEQ ID NO:93, SEQ ID NO:94, SEQ ID NO:95, SEQ ID NO:96, and SEQ ID NO:97.

In some embodiments, Staphylococcus aureus MU3 has a 16S RNA gene that is at least 90% identical to one or more (e.g., one, two, three, four, five, six, or all) of SEQ ID NO:98, SEQ ID NO:99, SEQ ID NO:100, SEQ ID NO:101, SEQ ID NO:102, SEQ ID NO:103, and SEQ ID NO:104. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, six, or all) of SEQ ID NO:98, SEQ ID NO:99, SEQ ID NO:100, SEQ ID NO:101, SEQ ID NO:102, SEQ ID NO:103, and SEQ ID NO:104.

In some embodiments, Staphylococcus aureus ACM 2434 has a 16S RNA gene that is at least 90% identical to one or more (e.g., one, two, three, four, five, six, seven, eight, nine, ten, eleven, twelve, thirteen, fourteen, fifteen, sixteen, seventeen, eighteen, or all) of SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, and SEQ ID NO:76. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, six, seven, eight, nine, ten, eleven, twelve, thirteen, fourteen, fifteen, sixteen, seventeen, eighteen, or all) of SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, and SEQ ID NO:76.

In some embodiments, Staphylococcus caprae 143.22 has a 16S RNA gene that is at least 90% identical to one or both of SEQ ID NO:105 and SEQ ID NO:106. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or both of SEQ ID NO:105 and SEQ ID NO:106.

In some embodiments, Staphylococcus caprae M23864 W1 has a 16S RNA gene that is at least 90% identical to one or more (e.g., one, two, three, or all) of SEQ ID NO:107, SEQ ID NO:108, SEQ ID NO:109, and SEQ ID NO:110. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, or all) of SEQ ID NO:107, SEQ ID NO:108, SEQ ID NO:109, and SEQ ID NO:110.

In some embodiments, Staphylococcus cohnii CCS 85 has a 16S RNA gene that is at least 90% identical to SEQ ID NO:111. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to SEQ ID NO:111.

In some embodiments, Staphylococcus devriesei KS SP60 has a 16S RNA gene that is at least 90% identical to SEQ ID NO:112. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to SEQ ID NO:112.

In some embodiments, Staphylococcus epidermidis WHO 12 has a 16S RNA gene that is at least 90% identical to one or more (e.g., one, two, three, four, five, six, or all) of SEQ ID NO:113, SEQ ID NO:114, SEQ ID NO:115, SEQ ID NO:116, SEQ ID NO:117, SEQ ID NO:118, and SEQ ID NO:119. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, six, or all) of SEQ ID NO:113, SEQ ID NO:114, SEQ ID NO:115, SEQ ID NO:116, SEQ ID NO:117, SEQ ID NO:118, and SEQ ID NO:119.

In some embodiments, Staphylococcus hominis DM 122 has a 16S RNA gene that is at least 90% identical to one or both of SEQ ID NO:120 and SEQ ID NO:121. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or both of SEQ ID NO:120 and SEQ ID NO:121.

In some embodiments, Streptococcus mitis R 360 has a 16S RNA gene that is at least 90% identical to one or more (e.g., one, two, three, or all) of SEQ ID NO:122, SEQ ID NO:123, SEQ ID NO:124, and SEQ ID NO:125. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, or all) of SEQ ID NO:122, SEQ ID NO:123, SEQ ID NO:124, and SEQ ID NO:125.

In some embodiments, Turicella otitidis 234/92 has a 16S RNA gene that is at least 90% identical to one or more (e.g., one, two, three, four, or all) SEQ ID NO:126, SEQ ID NO:127, SEQ ID NO:128, SEQ ID NO:129, and SEQ ID NO:130. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, or all) SEQ ID NO:126, SEQ ID NO:127, SEQ ID NO:128, SEQ ID NO:129, and SEQ ID NO:130.

In some embodiments, decreasing the population of an enriched (increased) bacterial strain can include administering a bacteriophage to the subject. See, for example, Sabino et al. Aliment Pharmacol Ther. 51(1):53-63, 2020. In some embodiments, decreasing the population of an enriched (increased) bacterial strain can include administering to the subject a composition comprising an effective amount of a bacterial strain (e.g., a bacterial strain selected from the group consisting of: Brevundimonas nasdae W1-2B, Capnocytophaga sputigena 4, Moraxella sp. LMG 5131, Neisseria elongata ATCC 29315, Staphylococcus felis GD521, Staphylococcus sciuri SC116, Streptococcus intermedius 393, and a combination thereof.

In some embodiments, methods provided herein can include administering the composition that includes an effective amount of one or more bacterial strains to the subject at least once per day. For example, the composition can be administered two, three, four, or more times per day. In some embodiments, an effective amount of the bacterial strain is administered in one dose, e.g., once per day. In some embodiments, an effective amount of the bacterial strain is administered in more than one dose, e.g., more than once per day. In some embodiments, the method comprises administering the composition to the subject daily, every other day, every three days, or once a week.

In some embodiments, an effective amount of a bacterial strain (e.g., Brevundimonas nasdae W1-2B, Capnocytophaga sputigena 4, Moraxella sp. LMG 5131, Neisseria elongata ATCC 29315, Staphylococcus felis GD521, Staphylococcus sciuri SC116, Streptococcus intermedius 393, or a combination thereof) in a composition described herein can include at least about 1×10³ CFUs of the bacterial strain. For example, an effective amount of a bacterial strain can be at least about 1×10³, about 1×10⁴, about 1×10⁵, about 1×10⁶, about 1×10⁷, about 1×10⁸, about 1×10⁹, about 1×10¹⁰, about 1×10¹¹, about 1×10¹², about 1×10¹³, or about 1×10¹⁴ CFUs of the bacterial strain. In some embodiments, the effective amount of a bacterial strain in a composition described herein comprises about 1×10³ to about 1×10¹⁵ CFUs of the bacterial strain (e.g., about 1×10³ to about 1×10⁶, about 1×10³ to about 1×10⁸, about 1×10³ to about 1×10¹⁰, about 1×10³ to about 1×10¹², about 1×10³ to about 1×10¹⁴, about 1×10⁷ to about 1×10¹², about 1×10¹³ to about 1×10¹⁵, about 1×10¹¹ to about 1×10¹⁵, about 1×10⁹ to about 1×10¹⁵, about 1×10⁷ to about 1×10¹⁵, or about 1×10⁵ to about 1×10¹⁵ CFUs of the bacterial strain).

In some embodiments, methods provided herein can include administering a composition comprising a bacterial strain as described herein in combination with one or more other treatments of atopic dermatits and/or in combination with adjunct therapies such as a therapeutic agent to inflammation. The composition comprising a bacterial strain and any other treatments and/or adjunct therapies can be administered together (e.g., in the same formulation), or the composition comprising the bacterial strain can be administered concurrently with, prior to, or subsequent to, the one or more other treatments or adjunct therapies.

In some embodiments, the treatment of atopic dermatits and/or adjunct therapy administered in combination with a composition comprising a bacterial strain as described herein comprises vitamin D, an antihistamine, a corticosteroid, a calcineurin inhibitor, an immunosuppressant, a phosphodiesterase-4 inhibitor, an anti-interleukin-4 receptor (IL-4Rα) antibody, an antibiotic, phototherapy, or a combination thereof. In some embodiments, the corticosteroid comprises hydrocortisone, clobetasol propionate, betamethasone dipropionate, halobetasol propionate, diflorasone diacetate, fluocinonide, halcinonide, amcinonide, desoximetasone, triamcinolone acetonide, mometasone furoate, fluticasone propionate, betamethasone dipropionate, halometasone, fluocinolone acetonide, hydrocortisone valerate, hydrocortisone butyrate, flurandrenolide, triamcinolone acetonide, mometasone furoate, fluticasone propionate, desonide, fluocinolone acetonide, hydrocortisone valerate, alclometasone dipropionate, triamcinolone acetonide, fluocinolone acetonide, desonide, or a combination thereof. In some embodiments, the treatment of atopic dermatits and/or adjunct therapy agent comprises diphenhydramine, tacrolimus, pimecrolimus, cyclosporine A, methotrexate, interferon gamma, mycophenolate mofetil, azathioprine, crisaborole, dupilumab, or a combination thereof.

In some embodiments, a prebiotic and/or probiotic can be administered in combination with a composition comprising a bacterial strain as described herein. Non-limiting examples of a probiotic include one of more of Bifidobacteria (e.g., B. animalis, B. breve, B. lactis, B. longum, B. longum, or B. infantis), Lactobacillus (e.g., L. acidophilus, L. reuteri, L. bulgaricus, L. lactis, L. casei, L. rhamnosus, L. plantarum, L. paracasei, or L. delbreuckii/bulgaricus), Saccharomyces boulardii, E. coli Nissle 1917, and Streptococcus thermophiles. Non-limiting examples of a prebiotic include a fructooligosaccharide (e.g., oligofructose, inulin, or an inulin-type fructan), a galactooligosaccharide, an amino acid, or an alcohol. See, for example, Ramirez-Farias et al. (2008. Br. J Nutr. 4:1-10) and Pool-Zobel and Sauer (2007. J Nutr. 137:2580-2584).

In some embodiments, an effective amount of the treatment of atopic dermatits and/or adjunct therapy is administered in combination with a composition comprising a bacterial strain as described herein.

In some embodiments, methods provided herein can include monitoring the subject after treatment with a composition described herein to determine if one or more symptoms have been alleviated, if the severity of one or more symptoms has been reduced, or if progression of the disease has been delayed or inhibited in the subject. There are numerous scores and clinical markers that can be utilized to assess the efficacy of administering a composition that includes bacterial strain as described herein in treating atopic dermatitis. Non-limiting examples of clinical scores that can be used to the efficacy of administering a composition that includes bacterial strain as described herein include the Eczema Area and Severity Index (EAST), Scoring Atopic Dermatitis (SCORAD), and Itch Numeric Rating Scale (NRS). Non-limiting examples of biomarkers include soluble CD30, serum interleukin-31 (IL-31), serum Th2 chemokines, serum CCL17, serum CCL22, and serum CCL27 (see, e.g., Eichenfield et al. J Am Acad Dermatol. 2014 February; 70(2): 338-351). In some embodiments, an improvement in one or more of the above indexes or biomarkers after administering a bacterial strain, or a composition thereof, as described herein to the subject indicates treatment of the atopic dermatitis.

In some embodiments, compositions provided herein can include one or more excipients and can be formulated for any of a number of delivery systems suitable for administration to a subject (e.g., probiotic or LBP delivery systems). Non-limiting examples of an excipient include a buffering agent, a diluent, a preservative, a stabilizer, a binding agent, a filler, a lubricant, a dispersion enhancer, a disintegrant, a lubricant, a disintegrant, a wetting agent, a glidant, a flavoring agent, a sweetener, and a coloring agent. For example, in some embodiments, tablets or capsules can be prepared by conventional means with excipients such as binding agents, fillers, lubricants, disintegrants, or wetting agents. In some embodiments, topical compositions can be prepared by conventional means with excipients. Any of the compositions described herein can be administered to a subject to treat atopic dermatitis as described herein.

In some embodiments, a composition as described herein can be formulated for oral delivery. In some embodiments, the composition can be formulated as a tablet, a chewable tablet, a capsule, a stick pack, a powder, effervescent powder, or a liquid. In some embodiments, a composition can include coated beads that contain the bacterial strain. In some embodiments, a powder comprising the bacterial strain can be suspended or dissolved in a drinkable liquid such as water for administration. In some embodiments, the composition is a solid composition.

In some embodiments, a composition described herein can be formulated for various immediate and controlled release profiles of the bacterial strain. For example, a controlled release formulation can include a controlled release coating disposed over the bacterial strain. In some embodiments, the controlled release coating is an enteric coating, a semi-enteric coating, a delayed release coating, or a pulsed release coating. In some embodiments, a coating can be suitable if it provides an appropriate lag in active release (i.e., release of the bacterial strain). For example, in some embodiments, the composition can be formulated as a tablet that includes a coating (e.g., an enteric coating).

In some embodiments, the composition can be formulated for topical delivery. In some embodiments, the composition can be in the form of a paste, gel, cream, spray, suppository, mousse, emollient, ointment, foam, or suspension.

In some embodiments, the bacterial strain in the composition is a culture of a single strain of organism. In some embodiments, the composition comprises a bacterial strain that is isolated. In some embodiments, the bacterial strain is isolated and cultured in vitro to increase the number or concentration of the bacterial strain. Increasing the number or concentration of the bacterial strain can be useful, for example, to enhance the efficacy of a composition comprising the bacterial strain.

In some embodiments, an effective amount of the bacterial strain in a composition described herein comprises at least about 1×10³ CFU of the bacterial strain. For example, at least about 1×10³, about 1×10⁴, about 1×10⁵, about 1×10⁶, about 1×10⁷, about 1×10⁸, about 1×10⁹, about 1×10¹⁰, about 1×10¹¹, about 1×10¹², about 1×10¹³, or about 1×10¹⁴ CFUs of the bacterial strain. In some embodiments, the effective amount of a bacterial strain in a composition described herein comprises about 1×10³ to about 1×10¹⁵ CFUs of the bacterial strain. For example, about 1×10³ to about 1×10⁶, about 1×10³ to about 1×10⁸, about 1×10³ to about 1×10¹⁰, about 1×10³ to about 1×10¹², about 1×10³ to about 1×10¹⁴, about 1×10⁷ to about 1×10¹², about 1×10¹³ to about 1×10¹⁵, about 1×10¹¹ to about 1×10¹⁵, about 1×10⁹ to about 1×10¹⁵, about 1×10⁷ to about 1×10¹⁵, or about 1×10⁵ to about 1×10¹⁵ CFUs of the bacterial strain.

In some embodiments, the composition can include one or more biologically pure strains (e.g., two or more, three or more, four or more, five or more, six or more, or seven bacterial strains). For example, the composition can include biologically pure Bacteroides eggerthii 1 2 48FAA, biologically pure Brevundimonas nasdae W1-2B, biologically pure Capnocytophaga sputigena 4, biologically pure Moraxella sp. LMG 5131, biologically pure Neisseria elongata ATCC 29315, biologically pure Staphylococcus felis GD521, biologically pure Staphylococcus sciuri SC116, biologically pure Streptococcus intermedius 393, or any combination thereof.

In some embodiments, the composition is a solid composition that includes at least 1×10³ CFUs of a bacterial strain (e.g., a biologically pure strain) and one or more excipients. Identifying characteristics of suitable strains, including homology to 16S rRNA sequences are described above.

In some embodiments, each member of the same bacterial strain has a 16S rRNA gene sequence with at least about 90%, about 91%, about 92%, about 93%, about 94%, about 95%, about 96%, about 97%, about 98%, about 99%, about 99.5%, about 99.6%, about 99.7%, about 99.8%, or about 99.9% sequence identity to the 16S rRNA gene sequence of each other member of the same bacterial strain.

In some embodiments, a bacterial strain in a composition described herein is preserved. Methods for preserving bacterial strains can include lyophilization and cryopreservation, optionally in the presence of a protectant. Non-limiting examples of protectants include sucrose, inulin, and glycerol. In some embodiments, a composition can include a lyophilized or cryopreserved bacterial strain such as Brevundimonas nasdae W1-2B, Capnocytophaga sputigena 4, Moraxella sp. LMG 5131, Neisseria elongata ATCC 29315, Staphylococcus felis GD521, Staphylococcus sciuri SC116, Streptococcus intermedius 393, or a combination thereof, and an optional protectant.

In some embodiments, wherein the bacterial strain is a combination of two or more of: Brevundimonas nasdae W1-2B, Capnocytophaga sputigena 4, Moraxella sp. LMG 5131, Neisseria elongata ATCC 29315, Staphylococcus felis GD521, Staphylococcus sciuri SC116, and Streptococcus intermedius 393 one or more of Brevundimonas nasdae W1-2B, Capnocytophaga sputigena 4, Moraxella sp. LMG 5131, Neisseria elongata ATCC 29315, Staphylococcus felis GD521, Staphylococcus sciuri SC116, and Streptococcus intermedius 393 are lyophilized or cryopreserved.

In some embodiments, the composition is a live bacterial product (LBP). In some embodiments, the bacterial strain in the composition is viable. The viable bacterial strain may be, for example, cryopreserved and/or lyophilized. In some embodiments, a composition for delivery of live bacterial strains (e.g., Brevundimonas nasdae W1-2B, Capnocytophaga sputigena 4, Moraxella sp. LMG 5131, Neisseria elongata ATCC 29315, Staphylococcus felis GD521, Staphylococcus sciuri SC116, Streptococcus intermedius 393, or a combination thereof), can be formulated to maintain viability of the bacterial strain. In some embodiments, the composition comprises elements that protect the bacterial strain from the acidic environment of the stomach (e.g., an enteric coating).

In some embodiments, wherein the bacterial strain is a combination of two or more of: Brevundimonas nasdae W1-2B, Capnocytophaga sputigena 4, Moraxella sp. LMG 5131, Neisseria elongata ATCC 29315, Staphylococcus felis GD521, Staphylococcus sciuri SC116, and Streptococcus intermedius 393 one or more of Brevundimonas nasdae W1-2B, Capnocytophaga sputigena 4, Moraxella sp. LMG 5131, Neisseria elongata ATCC 29315, Staphylococcus felis GD521, Staphylococcus sciuri SC116, and Streptococcus intermedius 393 are viable.

In some embodiments, the bacterial strain in the composition can be non-viable. In some embodiments, the non-viable bacterial strain is heat-killed, irradiated, or lysed.

In some embodiments, wherein the bacterial strain is a combination of two or more of: Brevundimonas nasdae W1-2B, Capnocytophaga sputigena 4, Moraxella sp. LMG 5131, Neisseria elongata ATCC 29315, Staphylococcus felis GD521, Staphylococcus sciuri SC116, and Streptococcus intermedius 393 one or more of Brevundimonas nasdae W1-2B, Capnocytophaga sputigena 4, Moraxella sp. LMG 5131, Neisseria elongata ATCC 29315, Staphylococcus felis GD521, Staphylococcus sciuri SC116, and Streptococcus intermedius 393 are non-viable (e.g., heat-killed, irradiated, or lysed).

In some embodiments, the bacterial strain as described herein may be used in prophylactic applications. For example, in a prophylactic application, a bacterial strain or a composition described herein can be administered to a subject susceptible to, or otherwise at risk of, a particular disease in an amount that is sufficient to at least partially reduce the risk of developing a disease. One of ordinary skill in the art will appreciate that the precise amounts of the bacterial strain administered may depend on a number of subject specific factors such as the subject's state of health and/or weight.

Also provided herein are methods for identifying a subject as having atopic dermatitis that include: (a) identifying a subject having a sample that has: (i) an increased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) bacterial species selected from: Corynebacterium genitalium, Corynebacterium matruchotii, Corynebacterium.striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; and/or (ii) a decreased level of one or more (e.g., two or more, three or more, four or more, five or more, six or more, or seven) bacterial species selected from: Brevundimonas nasdae, Capnocytophaga sputigena, Moraxella sp., Neisseria elongata, Staphylococcus felis, Staphylococcus sciuri, and Streptococcus intermedius; as having atopic dermatitis; or (b) identifying a subject having a sample that does not have: (i) an increased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) bacterial species selected from: Corynebacterium genitalium, Corynebacterium matruchotii, Corynebacterium.striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; or (ii) a decreased level of one or more (e.g., two or more, three or more, four or more, five or more, six or more, or seven) bacterial species selected from: Brevundimonas nasdae, Capnocytophaga sputigena, Moraxella sp., Neisseria elongata, Staphylococcus felis, Staphylococcus sciuri, and Streptococcus intermedius; as not having atopic dermatitis.

Also provided are methods for diagnosing a subject as having atopic dermatitis that include: (a) diagnosing a subject having a sample that has: (i) an increased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) bacterial species selected from: Corynebacterium genitalium, Corynebacterium matruchotii, Corynebacterium.striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; and/or (ii) a decreased level of one or more (e.g., two or more, three or more, four or more, five or more, six or more, or seven) bacterial species selected from: Brevundimonas nasdae, Capnocytophaga sputigena, Moraxella sp., Neisseria elongata, Staphylococcus felis, Staphylococcus sciuri, and Streptococcus intermedius; as having atopic dermatitis; or (b) diagnosing a subject having a sample that does not have: (i) an increased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) bacterial species selected from: Corynebacterium genitalium, Corynebacterium matruchotii, Corynebacterium.striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; or (ii) a decreased level of one or more (e.g., two or more, three or more, four or more, five or more, six or more, or seven) bacterial species selected from: Brevundimonas nasdae, Capnocytophaga sputigena, Moraxella sp., Neisseria elongata, Staphylococcus felis, Staphylococcus sciuri, and Streptococcus intermedius; as not having atopic dermatitis.

Also provided herein are methods of treating atopic dermatitis in a subject that include: (a) administering an atopic dermatitis therapy to a subject determined to have a sample that has: (i) an increased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) of: Corynebacterium genitalium, Corynebacterium matruchotii, Corynebacterium. striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; and/or (ii) a decreased level of one or more (e.g., two or more, three or more, four or more, five or more, six or more, or seven) of: Brevundimonas nasdae, Capnocytophaga sputigena, Moraxella sp., Neisseria elongata, Staphylococcus felis, Staphylococcus sciuri, and Streptococcus intermedius; or (b) not administering an atopic dermatitis therapy to a subject determined not to have a sample that has: (i) an increased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) of: Corynebacterium genitalium, Corynebacterium matruchotii, Corynebacterium.striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; and/or (ii) a decreased level of one or more (e.g., two or more, three or more, four or more, five or more, six or more, or seven) of: Brevundimonas nasdae, Capnocytophaga sputigena, Moraxella sp., Neisseria elongata, Staphylococcus felis, Staphylococcus sciuri, and Streptococcus intermedius.

Also provided herein are methods of treating atopic dermatitis in a subject, the method comprising: (a) administering a composition comprising an effective amount of a bacterial species selected from the group consisting of: Brevundimonas nasdae, Capnocytophaga sputigena, Moraxella sp., Neisseria elongata, Staphylococcus felis, Staphylococcus sciuri, and Streptococcus intermedius, and a combination thereof, as a monotherapy, or in conjunction with an atopic dermatitis therapy, to a subject determined to have a sample that has: (i) an increased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) of: Corynebacterium genitalium, Corynebacterium matruchotii, Corynebacterium.striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; and/or (ii) a decreased level of one or more (e.g., two or more, three or more, four or more, five or more, six or more, or seven) of: Brevundimonas nasdae, Capnocytophaga sputigena, Moraxella sp., Neisseria elongata, Staphylococcus felis, Staphylococcus sciuri, and Streptococcus intermedius; or (b) not administering a composition comprising an effective amount of a bacterial species selected from the group consisting of: Brevundimonas nasdae, Capnocytophaga sputigena, Moraxella sp., Neisseria elongata, Staphylococcus felis, Staphylococcus sciuri, and Streptococcus intermedius, and a combination thereof, to a subject determined not to have a sample that has: (i) an increased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) of: Corynebacterium genitalium, Corynebacterium matruchotii, Corynebacterium.striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; and/or (ii) a decreased level of one or more (e.g., two or more, three or more, four or more, five or more, six or more, or seven) of: Brevundimonas nasdae, Capnocytophaga sputigena, Moraxella, Neisseria elongata, Staphylococcus felis, Staphylococcus sciuri, and Streptococcus intermedius.

Some embodiments of these methods include detecting the level of one or more bacterial species in the sample from the subject. In some embodiments, the level of Corynebacterium genitalium, Corynebacterium matruchotii, Corynebacterium.striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, or Turicella otitidis is increased in comparison to the same bacterial species in a reference (control) sample.

In some embodiments, the method comprises determining that the sample has: (i) an increased level of two or more of: Corynebacterium genitalium, Corynebacterium matruchotii, Corynebacterium.striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; and/or (ii) a decreased level of two or more of: Brevundimonas nasdae, Capnocytophaga sputigena, Moraxella sp., Neisseria elongata, Staphylococcus felis, Staphylococcus sciuri, and Streptococcus intermedius.

In some embodiments, the method comprises determining that the sample has: (i) an increased level of three or more of: Corynebacterium genitalium, Corynebacterium matruchotii, Corynebacterium.striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; and/or (ii) a decreased level of two or more of: Brevundimonas nasdae, Capnocytophaga sputigena, Moraxella sp., Neisseria elongata, Staphylococcus felis, Staphylococcus sciuri, and Streptococcus intermedius.

In some embodiments, the method comprises determining that the sample has: (i) an increased level of four or more of: Corynebacterium genitalium, Corynebacterium matruchotii, Corynebacterium.striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; and/or (ii) a decreased level of two or more of: Brevundimonas nasdae, Capnocytophaga sputigena, Moraxella sp., Neisseria elongata, Staphylococcus felis, Staphylococcus sciuri, and Streptococcus intermedius.

The invention will be further described in the following examples, which do not limit the scope of the invention described in the claims.

### EXAMPLES

### Example 1. Identification of Bacterial Strains Associated with Atopic Dermatitis

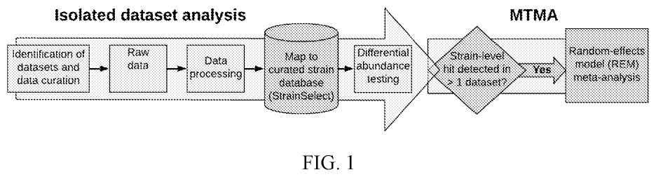

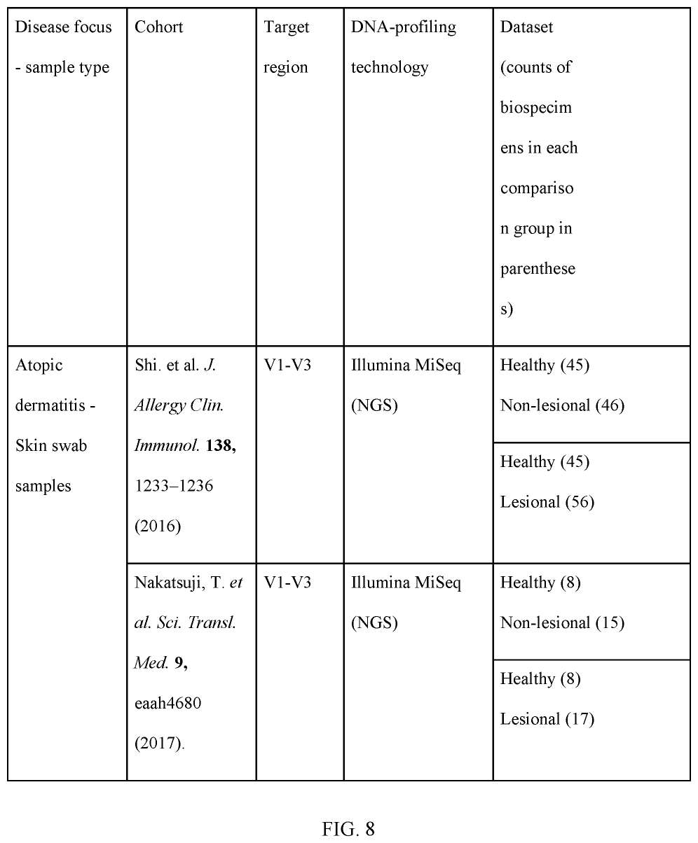

A multiple-technology meta-analysis (MTMA) approach was applied to datasets, which led to the identification of strains unique to atopic dermatitis. To overcome challenges of comparing clinical variables across datasets from multiple institutes, a controlled vocabulary of hierarchically organized terms and manually re-annotated metadata from public datasets using this vocabulary was created. FIG. 8 provides the details regarding the cohorts, datasets, and contrasts analyzed to identify differentially abundant (DA) taxa correlating with disease progression. Statistical analysis of each dataset was performed using the workflow described in FIG. 1. Strain-level annotation was achieved using StrainSelect, a database containing sequence information of bacterial and archaeal strains connected to genome identifiers, which facilitated comparative analysis of taxa abundances at a strain-level across datasets

### Methods

### Procurement of Raw Data and Metadata Curation

Fastq/Fasta files and metadata were procured from public repositories. Metadata stored with raw data, such as NCBI's Runlnfo table associated with the SRA Run Selector, and/or metadata published in tables in the primary text or supplementary files of the publication, were retrieved and manually re-annotated using a controlled vocabulary of hierarchically organized terms. An in-house database was created to store all study-related data and facilitate appropriate metadata annotation of all datasets via manual curation. Clinical metadata was stored in this database as a series of label:value pairs attached to the biospecimen from which the data files were generated.

### Processing, Strain Annotation and Statistical Analyses of Raw Data

16S rRNA Sequencing Datasets (16S-NGS and 16S-Sanger)

For NGS datasets, paired-end reads were merged and aligned to the StrainSelect database version 2014 (SS14) using USEARCH (usearch_global) (see, Edgar. Bioinformatics 26, 2460-2461 (2010)). All sequences matching a unique strain at an identity ≥99% were assigned a strain-level annotation. To ensure specificity of these strain matches, a difference of ≥0.25% between the identity of the best match and the second best match was required e.g., 99.75 vs. 99.5. StrainSelect is a repository of strain identifiers obtained from gene sequencing, genome sequencing, draft genomes, and metagenomic assemblies of known prokaryotic strains. Distinct strain matches counted to generate strain-level abundances. Remaining sequences were quality-filtered, chimera-filtered and clustered at ≥97% similarity via UPARSE (Allali et al. doi:10.1186/s12866-017-1101-8) to generated de-novo OTUs. OTU abundances were generated by aligning and counting all non-strain sequences against OTU representatives.

Downstream of a 5% prevalence filter, DESeq2 (Wood and Salzberg. Genome Biol. 15, R46 (2014)) was used to calculate significant differences in isolated analysis across all bins (OTUs and strains) and adjusted p-values were determined with the Benjamini-Hochberg correction. Significant results were determined as adjusted p-values <0.05. Log 2 fold change and standard error were calculated via DESeq2 and applied to subsequent analyses.

### Multi-Technology Meta-Analysis

Log 2 fold change and standard errors pertaining to per-dataset statistical results in each disease area were integrated in MTMA using a Random effects model (REM), generated using the metafor R package. Only bins with strain-level annotations in each dataset, and only those strains observed in at least two datasets, post prevalence filtering, were retained for REM analysis. False discovery correction for REM generated p-values was achieved using the Benjamini-Hochberg method. Differences are deemed to be statistically significant at adjusted (Benjamini-Hochberg corrected) p-values <0.05 in both isolated dataset analysis and MTMA.

### Results

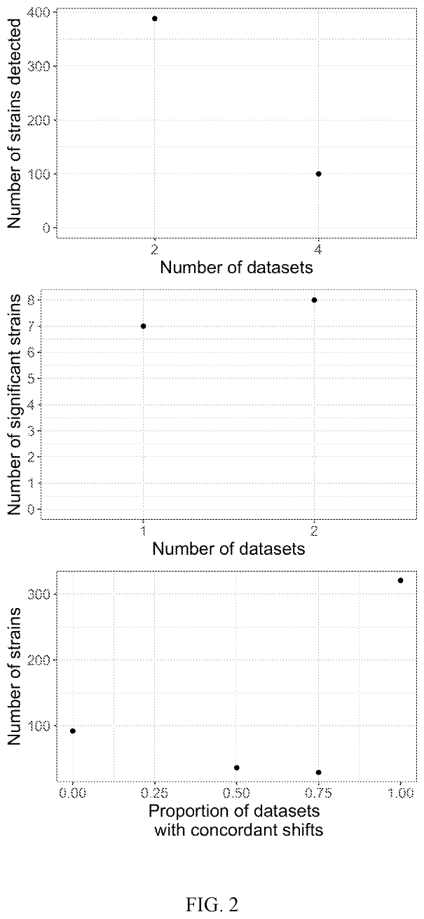

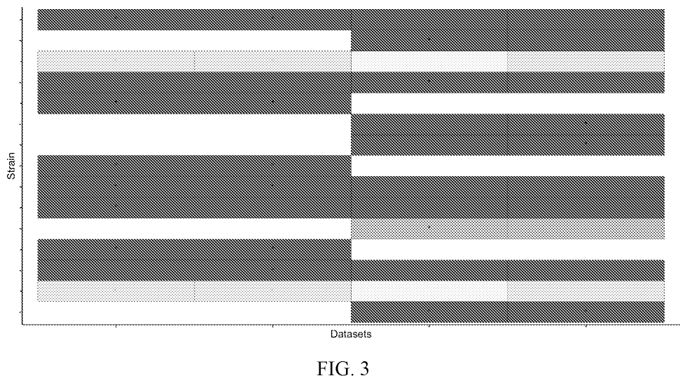

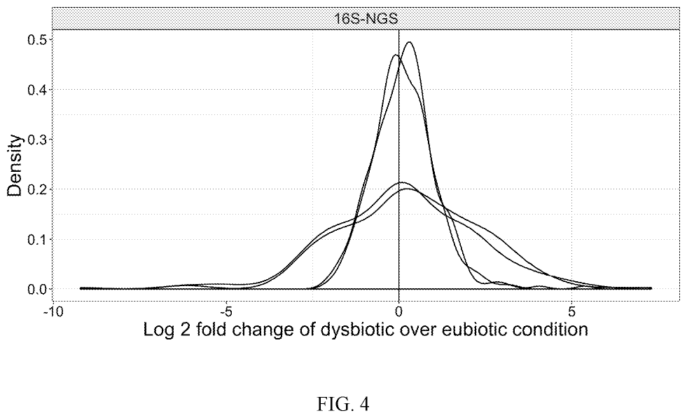

Identification of differentially abundant strains across cohorts from a simple comparison of isolated datasets was limited as less than 80% of the strains were detected across all datasets (FIG. 2, top panel). Further, while isolated analysis identified 15 strains as significantly differentially abundant, these strain-disease associations were cohort-specific (FIG. 2, middle panel) and in many cases not supported in trend, i.e., a strain being consistently associated with either homeostasis or dysbiosis, across cohorts (FIG. 3; light-shaded rows). Variation was also observed in the magnitude of differential abundance derived from the cohorts (log 2 fold change; FIG. 4). 13 of the 27 significantly differentially abundant strains were Staphylococci, confirming the known association of species in this genera, particularly the deleterious effect of S. aureus with atopic dermatitis. Interestingly, while previous work identified S. epidermidis and S. hominis species to be associated with a healthy skin microbiome³², we identified specific strains within these species that were enriched (increased) in AD and hence potentially disease promoting, highlighting that strain-identity even within species may be important to the role played by a bacterium in disease.

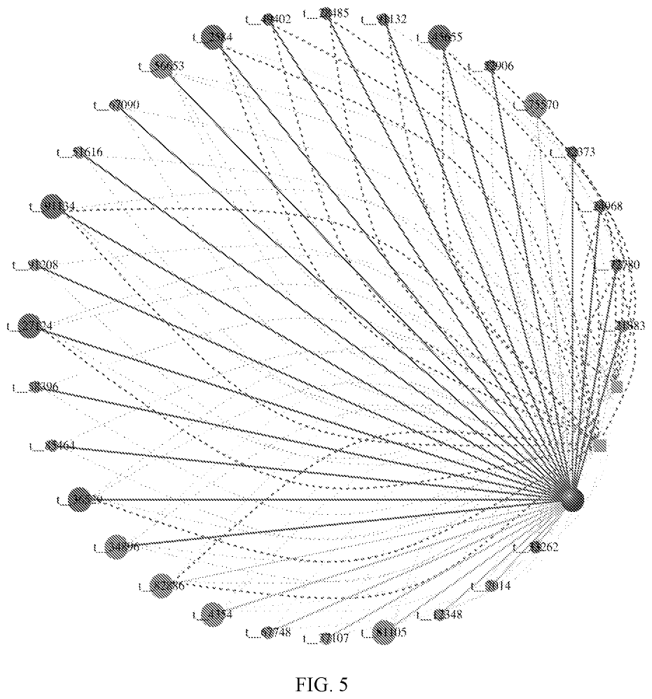

Strain-level results were integrated from isolated analyses in each disease via MTMA as described in FIG. 1. Significant associations were identified only when the direction of differential abundance of the strain was supported by multiple datasets (FIG. 5; blue strains connected to MTMA nodes via thick-solid lines). The associations in isolated analyses that were not supported in trend by other datasets were not significant by MTMA (FIG. 5; dark-green circles). Several strain-disease associations identified in MTMA were not identified in isolated analyses of the datasets (FIG. 5; purple dots). Thus, MTMA corroborates findings from isolated analysis if supported across datasets but eliminates if discordant, and MTMA identifies novel disease-strain associations that isolated analyses failed to detect.

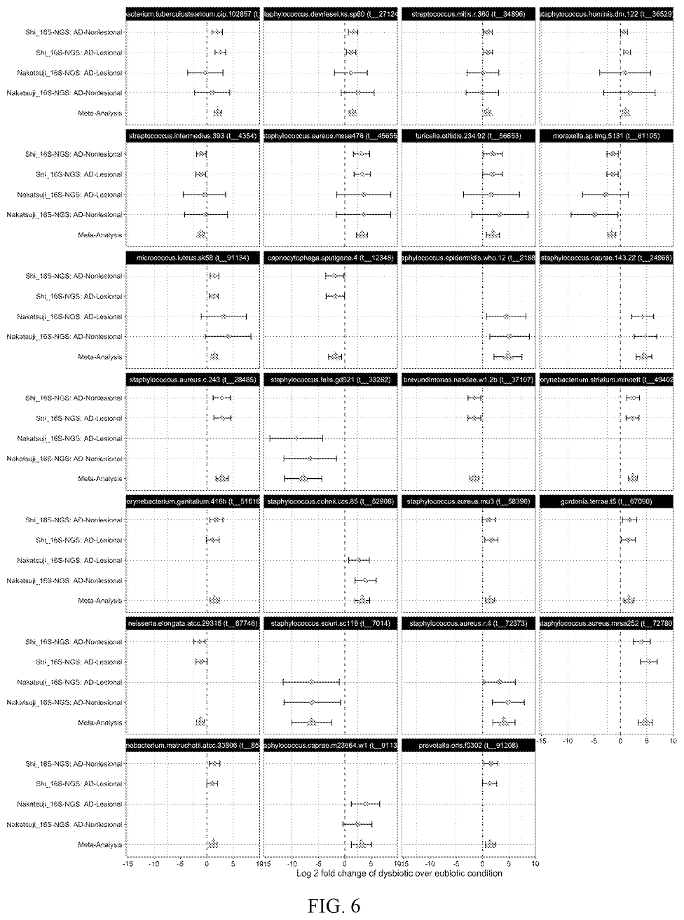

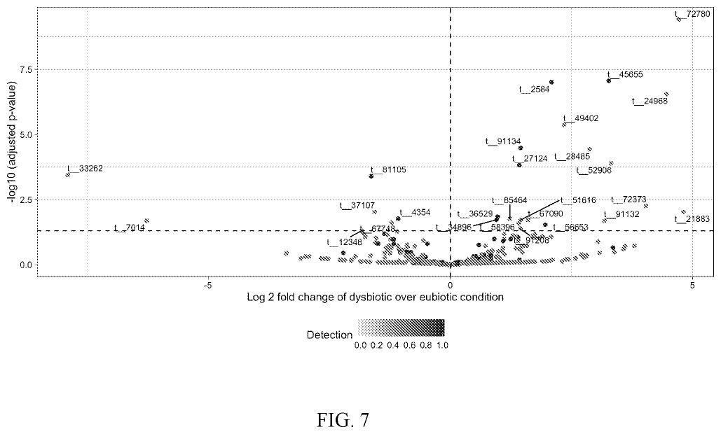

Enrichment (increase) of disease-associated bacteria in atopic dermatitis (FIG. 6), many of which were classified as belonging to pathogenic species, indicated that colonization by pathogens as opposed to a lack of homeostasis could be a key driver (FIG. 7).

MTMA can enable synthesis of existing knowledge of the microbiome, and the approach as shown in FIG. 1 can facilitate comparative analysis of taxa abundances at a strain-level across datasets generated with different DNA-profiling technologies. Harnessing the MTMA framework, with its ability to integrate datasets across DNA-profiling technologies and pinpoint specific strains, can allow for identification of robust microbiome modulators of disease by integrating the growing body of evidence on the role played by microbiome in disease.

### Other Embodiments

It is to be understood that while the invention has been described in conjunction with the detailed description thereof, the foregoing description is intended to illustrate and not limit the scope of the invention which is defined by the scope of the appended claims. Other aspects, advantages, and modification are within the scope of the following claims.

What is claimed is:

1. A method for identifying a subject as having atopic dermatitis, the method comprising:
(a) identifying a subject having a sample that has:
(i) an increased level of one or more bacterial species selected from: Corynebacterium genitalium, Corynebacterium matruchotii, Corynebacterium. striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; and/or
(ii) a decreased level of one or more bacterial species selected from: Brevundimonas nasdae, Capnocytophaga sputigena, Moraxella sp., Neisseria elongata, Staphylococcus felis, Staphylococcus sciuri, and Streptococcus intermedius;
as having atopic dermatitis; or
(b) identifying a subject having a sample that does not have:
(i) an increased level of one or more bacterial species selected from: Corynebacterium genitalium, Corynebacterium matruchotii, Corynebacterium.striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; or
(ii) a decreased level of one or more bacterial species selected from: Brevundimonas nasdae, Capnocytophaga sputigena, Moraxella sp., Neisseria elongata, Staphylococcus felis, Staphylococcus sciuri, and Streptococcus intermedius;
as not having atopic dermatitis.

2. A method for diagnosing a subject as having atopic dermatitis, the method comprising:
(a) diagnosing a subject having a sample that has:
(i) an increased level of one or more bacterial species selected from: Corynebacterium genitalium, Corynebacterium matruchotii, Corynebacterium.striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; and/or
(ii) a decreased level of one or more bacterial species selected from: Brevundimonas nasdae, Capnocytophaga sputigena, Moraxella sp., Neisseria elongata, Staphylococcus felis, Staphylococcus sciuri, and Streptococcus intermedius;
as having atopic dermatitis; or
(b) diagnosing a subject having a sample that does not have:
(i) an increased level of one or more bacterial species selected from: Corynebacterium genitalium, Corynebacterium matruchotii, Corynebacterium.striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; or
(ii) a decreased level of one or more bacterial species selected from: Brevundimonas nasdae, Capnocytophaga sputigena, Moraxella sp., Neisseria elongata, Staphylococcus felis, Staphylococcus sciuri, and Streptococcus intermedius;
as not having atopic dermatitis.

3. A method of treating atopic dermatitis in a subject, the method comprising:
(a) administering an atopic dermatitis therapy to a subject determined to have a sample that has:
(i) an increased level of one or more of: Corynebacterium genitalium, Corynebacterium matruchotii, Corynebacterium. striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; and/or
(ii) a decreased level of one or more of: Brevundimonas nasdae, Capnocytophaga sputigena, Moraxella sp., Neisseria elongata, Staphylococcus felis, Staphylococcus sciuri, and Streptococcus intermedius; or
(b) not administering an atopic dermatitis therapy to a subject determined not to have a sample that has:
(i) an increased level of one or more of: Corynebacterium genitalium, Corynebacterium matruchotii, Corynebacterium.striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; and/or
(ii) a decreased level of one or more of: Brevundimonas nasdae, Capnocytophaga sputigena, Moraxella sp., Neisseria elongata, Staphylococcus felis, Staphylococcus sciuri, and Streptococcus intermedius.

4. A method of treating atopic dermatitis in a subject, the method comprising:
(a) administering a composition comprising an effective amount of a bacterial species selected from the group consisting of: Brevundimonas nasdae, Capnocytophaga sputigena, Moraxella sp., Neisseria elongata, Staphylococcus felis, Staphylococcus sciuri, and Streptococcus intermedius, and a combination thereof, as a monotherapy, or in conjunction with an atopic dermatitis therapy, to a subject determined to have a sample that has:
(i) an increased level of one or more of: Corynebacterium genitalium, Corynebacterium matruchotii, Corynebacterium. striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; and/or
(ii) a decreased level of one or more of: Brevundimonas nasdae, Capnocytophaga sputigena, Moraxella sp., Neisseria elongata, Staphylococcus felis, Staphylococcus sciuri, and Streptococcus intermedius; or
(b) not administering a composition comprising an effective amount of a bacterial species selected from the group consisting of: Brevundimonas nasdae, Capnocytophaga sputigena, Moraxella sp., Neisseria elongata, Staphylococcus felis, Staphylococcus sciuri, and Streptococcus intermedius, and a combination thereof, to a subject determined not to have a sample that has:
(i) an increased level of one or more of: Corynebacterium genitalium, Corynebacterium matruchotii, Corynebacterium. striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; and/or
(ii) a decreased level of one or more of: Brevundimonas nasdae, Capnocytophaga sputigena, Moraxella, Neisseria elongata, Staphylococcus felis, Staphylococcus sciuri, and Streptococcus intermedius.

5. The method of any one of claims 1-4, wherein the method comprises detecting the level of one or more bacterial species in the sample from the subject.

6. The method of any one of claims 1-5, wherein the level of Corynebacterium genitalium, Corynebacterium matruchotii, Corynebacterium.striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, or Turicella otitidis is increased in comparison to the same bacterial species in a reference sample.

7. The method of any one of claims 1-6, wherein the method comprises determining that the sample has:
(i) an increased level of two or more of: Corynebacterium genitalium, Corynebacterium matruchotii, Corynebacterium.striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; and/or
(ii) a decreased level of two or more of: Brevundimonas nasdae, Capnocytophaga sputigena, Moraxella sp., Neisseria elongata, Staphylococcus felis, Staphylococcus sciuri, and Streptococcus intermedius.

8. The method of any one of claims 1-7, wherein the method comprises determining that the sample has:
(i) an increased level of three or more of: Corynebacterium genitalium, Corynebacterium matruchotii, Corynebacterium.striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; and/or
(ii) a decreased level of two or more of: Brevundimonas nasdae, Capnocytophaga sputigena, Moraxella sp., Neisseria elongata, Staphylococcus felis, Staphylococcus sciuri, and Streptococcus intermedius.

9. The method of any one of claims 1-8, wherein the method comprises determining that the sample has:
(i) an increased level of four or more of: Corynebacterium genitalium, Corynebacterium matruchotii, Corynebacterium.striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; and/or
(ii) a decreased level of two or more of: Brevundimonas nasdae, Capnocytophaga sputigena, Moraxella sp., Neisseria elongata, Staphylococcus felis, Staphylococcus sciuri, and Streptococcus intermedius.

10. The method of any one of claims 3 and 5-9, wherein the atopic dermatitis therapy comprises a probiotic.

11. The method of claims 3 and 5-10, wherein the atopic dermatitis therapy comprises vitamin D, an antihistamine, a corticosteroid, a calcineurin inhibitor, an immunosuppressant, a phosphodiesterase-4 inhibitor, an anti-interleukin-4 receptor (IL-4Rα) antibody, an antibiotic, phototherapy, or a combination thereof.

12. The method of claim 11, wherein the corticosteroid comprises hydrocortisone, clobetasol propionate, betamethasone dipropionate, halobetasol propionate, diflorasone diacetate, fluocinonide, halcinonide, amcinonide, desoximetasone, triamcinolone acetonide, mometasone furoate, fluticasone propionate, betamethasone dipropionate, halometasone, fluocinolone acetonide, hydrocortisone valerate, hydrocortisone butyrate, flurandrenolide, triamcinolone acetonide, mometasone furoate, fluticasone propionate, desonide, fluocinolone acetonide, hydrocortisone valerate, alclometasone dipropionate, triamcinolone acetonide, fluocinolone acetonide, desonide, or a combination thereof.

13. The method of claim 11 or 12, wherein the atopic dermatitis therapy comprises diphenhydramine, tacrolimus, pimecrolimus, cyclosporine A, methotrexate, interferon gamma, mycophenolate mofetil, azathioprine, crisaborole, dupilumab, or a combination thereof.

14. A method for treating a subject in need thereof, the method comprising administering to the subject a composition comprising an effective amount of a bacterial species selected from the group consisting of: Brevundimonas nasdae, Capnocytophaga sputigena, Moraxella sp., Neisseria elongata, Staphylococcus felis, Staphylococcus sciuri, and Streptococcus intermedius, and a combination thereof.

15. The method of claim 14, wherein the subject has atopic dermatitis.

16. A method for treating atopic dermatitis in a subject, the method comprising administering to the subject a composition comprising an effective amount of a bacterial species selected from the group consisting of: Brevundimonas nasdae, Capnocytophaga sputigena, Moraxella sp., Neisseria elongata, Staphylococcus felis, Staphylococcus sciuri, and Streptococcus intermedius, and a combination thereof.

17. A method for treating atopic dermatitis in a subject, the method comprising:
(a) detecting a dysbiosis associated with atopic dermatitis in a sample from the subject; and
(b) administering to the subject a composition comprising an effective amount of a bacterial species selected from the group consisting of: Brevundimonas nasdae, Capnocytophaga sputigena, Moraxella sp., Neisseria elongata, Staphylococcus felis, Staphylococcus sciuri, and Streptococcus intermedius, and a combination thereof.

18. The method of claim 17, wherein detecting the dysbiosis associated with atopic dermatitis comprises determining bacterial gene expression in the sample from the subject.

19. The method of claim 17 or 18, wherein detecting the dysbiosis associated with atopic dermatitis comprises determining bacterial composition in the sample from the subject.

20. The method of claim any one of claims 17-19, wherein detecting the dysbiosis associated with atopic dermatitis comprises determining that Corynebacterium genitalium, Corynebacterium matruchotii, Corynebacterium.striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, Turicella otitidis, or a combination thereof, is increased in the sample from subject.

21. The method of claim any one of claims 17-20, wherein detecting the dysbiosis associated with atopic dermatitis comprises determining that Brevundimonas nasdae, Capnocytophaga sputigena, Moraxella sp., Neisseria elongata, Staphylococcus felis, Staphylococcus sciuri, Streptococcus intermedius, or a combination thereof, is decreased in the sample from subject.

22. The method of claim 21, wherein Brevundimonas nasdae, Capnocytophaga sputigena, Moraxella sp., Neisseria elongata, Staphylococcus felis, Staphylococcus sciuri, Streptococcus intermedius, or a combination thereof, is decreased in the gastrointestinal tract of the subject.

23. The method of any one of claims 1-13 and 17-22, wherein the sample is a skin swab sample.

24. A method for treating a subject in need thereof, the method comprising decreasing a population of an increased bacterial strain in the subject, wherein the increased bacterial strain is selected from the group consisting of: Corynebacterium genitalium, Corynebacterium matruchotii, Corynebacterium.striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, Turicella otitidis, and a combination thereof.

25. The method of claim 24, wherein the subject has atopic dermatitis.

26. The method of claim 24 or 25, wherein decreasing the population of an increased bacterial strain comprises administering to the subject a bacteriophage.

27. The method of any one of claims 24-26, wherein decreasing the population of an increased bacterial strain comprises administering to the subject a composition comprising an effective amount of a bacterial species selected from the group consisting of: Brevundimonas nasdae, Capnocytophaga sputigena, Moraxella sp., Neisseria elongata, Staphylococcus felis, Staphylococcus sciuri, Streptococcus intermedius, and a combination thereof.

28. The method of any one of claims 1-13 and 20-27, wherein the bacterial species Corynebacterium genitalium comprises the bacterial strain Corynebacterium genitalium 418H.

29. The method of any one of claims 1-13 and 20-28, wherein the bacterial species Corynebacterium matruchotii comprises the bacterial strain Corynebacterium matruchotii ATCC 33806.

30. The method of any one of claims 1-13 and 20-29, wherein the bacterial species Corynebacterium.striatum comprises the bacterial strain Corynebacterium striatum ATCC 6940.

31. The method of any one of claims 1-13 and 20-30, wherein the bacterial species Corynebacterium matruchotii comprises the bacterial strain Corynebacterium tuberculostearicum CIP 102857.

32. The method of any one of claims 1-13 and 20-31, wherein the bacterial species Finegoldia magna comprises the bacterial strain Finegoldia magna 312.

33. The method of any one of claims 1-13 and 20-32, wherein the bacterial species Gordonia terrae comprises the bacterial strain Gordonia terrae T5.

34. The method of any one of claims 1-13 and 20-33, wherein the bacterial species Micrococcus luteus comprises the bacterial strain Micrococcus luteus SK58.

35. The method of any one of claims 1-13 and 20-34, wherein the bacterial species Peptococcus saccharolyticus comprises the bacterial strain Peptococcus saccharolyticus S1.

36. The method of any one of claims 1-13 and 20-35, wherein the bacterial species Prevotella oris comprises the bacterial strain Prevotella oris F0302.

37. The method of any one of claims 1-13 and 20-36, wherein the bacterial species Staphylococcus aureus comprises the bacterial strain Staphylococcus aureus C-243, Staphylococcus aureus MRSA252, Staphylococcus aureus MSSA476, Staphylococcus aureus MU3, Staphylococcus aureus ACM 2434, or a combination thereof.

38. The method of any one of claims 1-13 and 20-37, wherein the bacterial species Staphylococcus caprae comprises the bacterial strain Staphylococcus caprae 143.22, Staphylococcus caprae M23864 W1, or a combination thereof.

39. The method of any one of claims 1-13 and 20-38, wherein the bacterial species Staphylococcus cohnii comprises the bacterial strain Staphylococcus cohnii CCS 85.

40. The method of any one of claims 1-13 and 20-39, wherein the bacterial species Staphylococcus devriesei comprises the bacterial strain Staphylococcus devriesei KS SP60.

41. The method of any one of claims 1-13 and 20-40, wherein the bacterial species Staphylococcus epidermidis comprises the bacterial strain Staphylococcus epidermidis WHO 12.

42. The method of any one of claims 1-13 and 20-41, wherein the bacterial species Staphylococcus hominis comprises the bacterial strain Staphylococcus hominis DM 122.

43. The method of any one of claims 1-13 and 20-42, wherein the bacterial species Streptococcus mitis comprises the bacterial strain Streptococcus mitis R 360.

44. The method of any one of claims 1-13 and 20-43, wherein the bacterial species Turicella otitidis comprises the bacterial strain Turicella otitidis 234/92.

45. The method of any one of claims 1-13 and 20-44, wherein the bacterial species Brevundimonas nasdae comprises the bacterial strain Brevundimonas nasdae W1-2B.

46. The method of any one of claims 1-13 and 20-45, wherein the bacterial species Capnocytophaga sputigena comprises the bacterial strain Capnocytophaga sputigena 4.

47. The method of any one of claims 1-13 and 20-46, wherein the bacterial species Moraxella sp. comprises the bacterial strain Moraxella sp. LMG 5131.

48. The method of any one of claims 1-13 and 20-47, wherein the bacterial species Neisseria elongata comprises the bacterial strain Neisseria elongata ATCC 29315.

49. The method of any one of claims 1-13 and 20-48, wherein the bacterial species Staphylococcus felis comprises the bacterial strain Staphylococcus felis GD521.

50. The method of any one of claims 1-13 and 20-49, wherein the bacterial species Staphylococcus sciuri comprises the bacterial strain Staphylococcus sciuri SC116.

51. The method of any one of claims 1-13 and 20-50, wherein the bacterial species Streptococcus intermedius comprises the bacterial strain Streptococcus intermedius 393.

52. The method of any one of claims 45-51, wherein the Brevundimonas nasdae W1-2B has a 16S RNA gene that is at least 95% identical to SEQ ID NO:1.

53. The method of any one of claims 46-52, wherein the Capnocytophaga sputigena 4 has a 16S RNA gene that is at least 95% identical to SEQ ID NO:2.

54. The method of any one of claims 46-52, wherein the Capnocytophaga sputigena 4 has a 16S RNA gene that is at least 95% identical to SEQ ID NO:3.

55. The method of any one of claims 46-52, wherein the Capnocytophaga sputigena 4 has a 16S+RNA gene that is at least 95% identical to SEQ ID NO:4.

56. The method of any one of claims 46-52, wherein the Capnocytophaga sputigena 4 has a 16S RNA gene that is at least 95% identical to SEQ ID NO:5.

57. The method of any one of claims 46-52, wherein the Capnocytophaga sputigena 4 has a 16S RNA gene that is at least 95% identical to SEQ ID NO:6.

58. The method of any one of claims 47-57, wherein the Moraxella sp. LMG 5131 has a 16S RNA gene that is at least 95% identical to SEQ ID NO:7.

59. The method of any one of claims 47-57, wherein the Moraxella sp. LMG 5131 has a 16S RNA gene that is at least 95% identical to SEQ ID NO:8.

60. The method of any one of claims 47-57, wherein the Moraxella sp. LMG 5131 has a 16S RNA gene that is at least 95% identical to SEQ ID NO:9.

61. The method of any one of claims 47-57, wherein the Moraxella sp. LMG 5131 has a 16S RNA gene that is at least 95% identical to SEQ ID NO:10.

62. The method of any one of claims 47-57, wherein the Moraxella sp. LMG 5131 has a 16S RNA gene that is at least 95% identical to SEQ ID NO:11.

63. The method of any one of claims 48-62, wherein the Neisseria elongata ATCC 29315 has a 16S RNA gene that is at least 95% identical to SEQ ID NO:12.

64. The method of any one of claims 48-62, wherein the Neisseria elongata ATCC 29315 has a 16S RNA gene that is at least 95% identical to SEQ ID NO:13.

65. The method of any one of claims 48-62, wherein the Neisseria elongata ATCC 29315 has a 16S RNA gene that is at least 95% identical to SEQ ID NO:14.

66. The method of any one of claims 48-62, wherein the Neisseria elongata ATCC 29315 has a 16S RNA gene that is at least 95% identical to SEQ ID NO:15.

67. The method of any one of claims 48-62, wherein the Neisseria elongata ATCC 29315 has a 16S RNA gene that is at least 95% identical to SEQ ID NO:16.

68. The method of any one of claims 48-62, wherein the Neisseria elongata ATCC 29315 has a 16S RNA gene that is at least 95% identical to SEQ ID NO:17.

69. The method of any one of claims 49-68, wherein the Staphylococcus fells GD521 has a 16S RNA gene that is at least 95% identical to SEQ ID NO:18.

70. The method of any one of claims 50-69, wherein the Staphylococcus sciuri SC116 has a 16S RNA gene that is at least 95% identical to SEQ ID NO:19.

71. The method of any one of claims 51-70, wherein the Streptococcus intermedius 393 has a 16S RNA gene that is at least 95% identical to SEQ ID NO:20.

72. The method of any one of claims 51-70, wherein the Streptococcus intermedius 393 has a 16S RNA gene that is at least 95% identical to SEQ ID NO:21.

73. The method of any one of claims 51-70, wherein the Streptococcus intermedius 393 has a 16S RNA gene that is at least 95% identical to SEQ ID NO:22.

74. The method of any one of claims 51-70, wherein the Streptococcus intermedius 393 has a 16S RNA gene that is at least 95% identical to SEQ ID NO:23.

75. The method of any one of claims 51-70, wherein the Streptococcus intermedius 393 has a 16S RNA gene that is at least 95% identical to SEQ ID NO:24.

76. The method of any one of claims 51-70, wherein the Streptococcus intermedius 393 has a 16S RNA gene that is at least 95% identical to SEQ ID NO:25.

77. The method of any one of claims 51-70, wherein the Streptococcus intermedius 393 has a 16S RNA gene that is at least 95% identical to SEQ ID NO:26.

78. The method of any one of claims 51-70, wherein the Streptococcus intermedius 393 has a 16S RNA gene that is at least 95% identical to SEQ ID NO:27.

79. The method of any one of claims 51-70, wherein the Streptococcus intermedius 393 has a 16S RNA gene that is at least 95% identical to SEQ ID NO:28.

80. The method of any one of claims 51-70, wherein the Streptococcus intermedius 393 has a 16S RNA gene that is at least 95% identical to SEQ ID NO:29.

81. The method of any one of claims 51-70, wherein the Streptococcus intermedius 393 has a 16S RNA gene that is at least 95% identical to SEQ ID NO:30.

82. The method of any one of claims 4, 14-23, and 27-81, wherein administering the composition improves epidermal barrier function of the subject.

83. The method of any one of claims 4, 14-23, and 27-82, wherein the bacterial species in the composition is viable.

84. The method of any one of claims 4, 14-23, and 27-83, wherein the bacterial strain is lyophilized.

85. The method of any one of claims 4, 14-23, and 27-84, wherein the composition further comprises one or more cryopreservants.

86. The method of any one of claims 4, 14-23, and 27-85, wherein the effective amount of the bacterial strain comprises at least about 1×10³ colony forming units (CFU) of the bacterial strain.

87. The method of any one of claims 4, 14-23, and 27-86, wherein the effective amount of the bacterial strain comprises about 1×10⁴ to about 1×10¹⁵ CFU of the bacterial strain.

88. The method of any one of claims 4, 14-23, and 27-87, wherein the effective amount of the bacterial strain comprises about 1×10⁶ to about 1×10¹⁰ CFU of the bacterial strain.

89. The method of any one of claims 4, 14-23, and 27-82, wherein the bacterial strain in the composition is non-viable.

90. The method of claim 89, wherein the non-viable bacterial strain is heat-killed, irradiated, or lysed.

91. The method of any one of claims 4, 14-23, and 27-90, wherein the method comprises administering the composition to the subject once, twice, or three times per day.

92. The method of any one of claims 4, 14-23, and 27-91, wherein the composition is formulated for topical administration.

93. The method of any one of claims 4, 14-23, and 27-92, wherein the composition is formulated as a paste, gel, cream, spray, mousse, emollient, ointment, foam, or suspension.

94. The method of any one of claims 4, 14-23, and 27-91, wherein the composition is formulated for oral administration.

95. The method of any one of claims 4, 14-23, and 27-91, wherein the composition is formulated for rectal administration.

96. The method of any one of claims 4, 14-23, 27-91, 94, and 95, wherein the composition is formulated as a tablet, a capsule, a powder, or a liquid.

97. The method of any one of claims 4, 14-23, 27-91, and 94-96, wherein the composition is formulated as a tablet.

98. The method of claim 97, wherein the tablet is coated.

99. The method of claim 98, wherein the coating comprises an enteric coating.

100. The method of any one of claims 14-99, wherein the method further comprises administering another atopic dermatitis therapy and/or adjunct therapy to the subject.

101. The method of claim 100, wherein the composition comprising the bacterial strain and the atopic dermatitis therapy and/or adjunct therapy are administered simultaneously.

102. The method of claim 100, wherein the composition comprising the bacterial strain and theatopic dermatitis therapy and/or adjunct therapy are administered sequentially.

103. The method of claim 100, wherein the composition comprising the bacterial strain further comprises the atopic dermatitis therapy and/or adjunct therapy.

104. The method of any one of claims 100-103, wherein the atopic dermatitis therapy and/or adjunct therapy comprises a probiotic.

105. The method of claim 100-104, wherein the atopic dermatitis therapy and/or adjunct therapy comprises vitamin D, an antihistamine, a corticosteroid, a calcineurin inhibitor, an immunosuppressant, a phosphodiesterase-4 inhibitor, an anti-interleukin-4 receptor (IL-4Rα) antibody, an antibiotic, phototherapy, or a combination thereof.

106. The method of claim 105, wherein the corticosteroid comprises hydrocortisone, clobetasol propionate, betamethasone dipropionate, halobetasol propionate, diflorasone diacetate, fluocinonide, halcinonide, amcinonide, desoximetasone, triamcinolone acetonide, mometasone furoate, fluticasone propionate, betamethasone dipropionate, halometasone, fluocinolone acetonide, hydrocortisone valerate, hydrocortisone butyrate, flurandrenolide, triamcinolone acetonide, mometasone furoate, fluticasone propionate, desonide, fluocinolone acetonide, hydrocortisone valerate, alclometasone dipropionate, triamcinolone acetonide, fluocinolone acetonide, desonide, or a combination thereof.

107. The method of claim 105 or 106, wherein the atopic dermatitis therapy and/or adjunct therapy comprises diphenhydramine, tacrolimus, pimecrolimus, cyclosporine A, methotrexate, interferon gamma, mycophenolate mofetil, azathioprine, crisaborole, dupilumab, or a combination thereof.

108. The method of any one of claims 1-107, wherein the subject is a human.

109. A method for treating a subject in need thereof, the method comprising administering to the subject a composition comprising an effective amount of a bacterial strain selected from the group consisting of: Brevundimonas nasdae W1-2B, Capnocytophaga sputigena 4, Moraxella sp. LMG 5131, Neisseria elongata ATCC 29315, Staphylococcus felis GD521, Staphylococcus sciuri SC116, Streptococcus intermedius 393, and a combination thereof.

110. The method of claim 109, wherein the subject has atopic dermatitis.

111. A method for treating atopic dermatitis in a subject, the method comprising administering to the subject a composition comprising an effective amount of a bacterial strain selected from the group consisting of: Brevundimonas nasdae W1-2B, Capnocytophaga sputigena 4, Moraxella sp. LMG 5131, Neisseria elongata ATCC 29315, Staphylococcus felis GD521, Staphylococcus sciuri SC116, Streptococcus intermedius 393, and a combination thereof.

112. A method for treating atopic dermatitis in a subject, the method comprising:
(a) detecting a dysbiosis associated with atopic dermatitis in a sample from the subject; and
(b) administering to the subject a composition comprising an effective amount of a bacterial strain selected from the group consisting of: Brevundimonas nasdae W1-2B, Capnocytophaga sputigena 4, Moraxella sp. LMG 5131, Neisseria elongata ATCC 29315, Staphylococcus felis GD521, Staphylococcus sciuri SC116, Streptococcus intermedius 393, and a combination thereof.

113. The method of claim 112, wherein the sample is a skin swab sample.

114. The method of claim 112 or 113, wherein detecting the dysbiosis associated with atopic dermatitis comprises determining bacterial gene expression in the sample from the subject.

115. The method of any one of claims 112-114, wherein detecting the dysbiosis associated with atopic dermatitis comprises determining bacterial composition in the sample from the subject.

116. The method of claim any one of claims 112-115, wherein detecting the dysbiosis associated with atopic dermatitis comprises determining that Corynebacterium genitalium 418H, Corynebacterium matruchotii ATCC 33806, Corynebacterium.striatum ATCC 6940, Corynebacterium tuberculostearicum CIP 102857, Finegoldia magna 312, Gordonia terrae T5, Micrococcus luteus SK58, Peptococcus saccharolyticus S1, Prevotella oris F0302, Staphylococcus aureus C-243, Staphylococcus aureus MRSA252, Staphylococcus aureus MSSA476, Staphylococcus aureus MU3, Staphylococcus aureus ACM 2434, Staphylococcus caprae 143.22, Staphylococcus caprae M23864 W1, Staphylococcus cohnii CCS 85, Staphylococcus devriesei KS SP60, Staphylococcus epidermidis WHO 12, Staphylococcus hominis DM 122, Streptococcus mitis R 360, Turicella otitidis 234/92, or a combination thereof, is increased in the sample from subject.

117. The method of claim any one of claims 112-116, wherein detecting the dysbiosis associated with atopic dermatitis comprises determining that Brevundimonas nasdae W1-2B, Capnocytophaga sputigena 4, Moraxella sp. LMG 5131, Neisseria elongata ATCC 29315, Staphylococcus felis GD521, Staphylococcus sciuri SC116, Streptococcus intermedius 393, or a combination thereof, is decreased in the sample from subject.

118. The method of claim 117, wherein Brevundimonas nasdae W1-2B, Capnocytophaga sputigena 4, Moraxella sp. LMG 5131, Neisseria elongata ATCC 29315, Staphylococcus felis GD521, Staphylococcus sciuri SC116, Streptococcus intermedius 393, or a combination thereof, is decreased in the gastrointestinal tract of the subject.

119. A method for treating a subject in need thereof, the method comprising decreasing a population of an increased bacterial strain in the subject, wherein the increased bacterial strain is selected from the group consisting of: Corynebacterium genitalium 418H, Corynebacterium matruchotii ATCC 33806, Corynebacterium.striatum ATCC 6940, Corynebacterium tuberculostearicum CIP 102857, Finegoldia magna 312, Gordonia terrae T5, Micrococcus luteus SK58, Peptococcus saccharolyticus S1, Prevotella oris F0302, Staphylococcus aureus C-243, Staphylococcus aureus MRSA252, Staphylococcus aureus MSSA476, Staphylococcus aureus MU3, Staphylococcus aureus ACM 2434, Staphylococcus caprae 143.22, Staphylococcus caprae M23864 W1, Staphylococcus cohnii CCS 85, Staphylococcus devriesei KS SP60, Staphylococcus epidermidis WHO 12, Staphylococcus hominis DM 122, Streptococcus mitis R 360, Turicella otitidis 234/92, and a combination thereof.

120. The method of claim 119, wherein the subject has atopic dermatitis.

121. The method of claim 119 or 120, wherein decreasing the population of an increased bacterial strain comprises administering to the subject a bacteriophage.

122. The method of any one of claims 119-121, wherein decreasing the population of an increased bacterial strain comprises administering to the subject a composition comprising an effective amount of a bacterial strain selected from the group consisting of: Brevundimonas nasdae W1-2B, Capnocytophaga sputigena 4, Moraxella sp. LMG 5131, Neisseria elongata ATCC 29315, Staphylococcus felis GD521, Staphylococcus sciuri SC116, Streptococcus intermedius 393, and a combination thereof.

123. The method of any one of claims 109-118 and 122, wherein the bacterial strain comprises Brevundimonas nasdae W1-2B.

124. The method of any one of claims 109-118, 122, and 123, wherein the bacterial strain comprises Capnocytophaga sputigena 4.

125. The method of any one of claims 109-118 and 122-124, wherein the bacterial strain comprises Moraxella sp. LMG 5131.

126. The method of any one of claims 109-118 and 122-125, wherein the bacterial strain comprises Neisseria elongata ATCC 29315.

127. The method of any one of claims 109-118 and 122-126, wherein the bacterial strain comprises Staphylococcus felis GD521.

128. The method of any one of claims 109-118 and 122-127, wherein the bacterial strain comprises Staphylococcus sciuri SC116.

129. The method of any one of claims 109-118 and 122-128, wherein the bacterial strain comprises Streptococcus intermedius 393.

130. The method of any one of claims 109-118 and 122-129, wherein the bacterial strain improves epidermal barrier function of the subject.

131. The method of any one of claims 109-118 and 122-130, wherein the Brevundimonas nasdae W1-2B has a 16S RNA gene that is at least 95% identical to SEQ ID NO:1.

132. The method of any one of claims 109-118 and 122-131, wherein the Capnocytophaga sputigena 4 has a 16S RNA gene that is at least 95% identical to SEQ ID NO:2.

133. The method of any one of claims 109-118 and 122-132, wherein the Capnocytophaga sputigena 4 has a 16S RNA gene that is at least 95% identical to SEQ ID NO:3.

134. The method of any one of claims 109-118 and 122-133, wherein the Capnocytophaga sputigena 4 has a 16S RNA gene that is at least 95% identical to SEQ ID NO:4.

135. The method of any one of claims 109-118 and 122-134, wherein the Capnocytophaga sputigena 4 has a 16S RNA gene that is at least 95% identical to SEQ ID NO:5.

136. The method of any one of claims 109-118 and 122-135, wherein the Capnocytophaga sputigena 4 has a 16S RNA gene that is at least 95% identical to SEQ ID NO:6.

137. The method of any one of claims 109-118 and 122-136, wherein the Moraxella sp. LMG 5131 has a 16S RNA gene that is at least 95% identical to SEQ ID NO:7.

138. The method of any one of claims 109-118 and 122-137, wherein the Moraxella sp. LMG 5131 has a 16S RNA gene that is at least 95% identical to SEQ ID NO:8.

139. The method of any one of claims 109-118 and 122-138, wherein the Moraxella sp. LMG 5131 has a 16S RNA gene that is at least 95% identical to SEQ ID NO:9.

140. The method of any one of claims 109-118 and 122-139, wherein the Moraxella sp. LMG 5131 has a 16S RNA gene that is at least 95% identical to SEQ ID NO:10.

141. The method of any one of claims 109-118 and 122-140, wherein the Moraxella sp. LMG 5131 has a 16S RNA gene that is at least 95% identical to SEQ ID NO:11.

142. The method of any one of claims 109-118 and 122-141, wherein the Neisseria elongata ATCC 29315 has a 16S RNA gene that is at least 95% identical to SEQ ID NO:12.

143. The method of any one of claims 109-118 and 122-142, wherein the Neisseria elongata ATCC 29315 has a 16S RNA gene that is at least 95% identical to SEQ ID NO:13.

144. The method of any one of claims 109-118 and 122-143, wherein the Neisseria elongata ATCC 29315 has a 16S RNA gene that is at least 95% identical to SEQ ID NO:14.

145. The method of any one of claims 109-118 and 122-144, wherein the Neisseria elongata ATCC 29315 has a 16S RNA gene that is at least 95% identical to SEQ ID NO:15.

146. The method of any one of claims 109-118 and 122-145, wherein the Neisseria elongata ATCC 29315 has a 16S RNA gene that is at least 95% identical to SEQ ID NO:16.

147. The method of any one of claims 109-118 and 122-146, wherein the Neisseria elongata ATCC 29315 has a 16S RNA gene that is at least 95% identical to SEQ ID NO:17.

148. The method of any one of claims 109-118 and 122-147, wherein the Staphylococcus felis GD521 has a 16S RNA gene that is at least 95% identical to SEQ ID NO:18.

149. The method of any one of claims 109-118 and 122-148, wherein the Staphylococcus sciuri SC116 has a 16S RNA gene that is at least 95% identical to SEQ ID NO:19.

150. The method of any one of claims 109-118 and 122-149, wherein the Streptococcus intermedius 393 has a 16S RNA gene that is at least 95% identical to SEQ ID NO:20.

151. The method of any one of claims 109-118 and 122-150, wherein the Streptococcus intermedius 393 has a 16S RNA gene that is at least 95% identical to SEQ ID NO:21.

152. The method of any one of claims 109-118 and 122-151, wherein the Streptococcus intermedius 393 has a 16S RNA gene that is at least 95% identical to SEQ ID NO:22.

153. The method of any one of claims 109-118 and 122-152, wherein the Streptococcus intermedius 393 has a 16S RNA gene that is at least 95% identical to SEQ ID NO:23.

154. The method of any one of claims 109-118 and 122-153, wherein the Streptococcus intermedius 393 has a 16S RNA gene that is at least 95% identical to SEQ ID NO:24.

155. The method of any one of claims 109-118 and 122-154, wherein the Streptococcus intermedius 393 has a 16S RNA gene that is at least 95% identical to SEQ ID NO:25.

156. The method of any one of claims 109-118 and 122-155, wherein the Streptococcus intermedius 393 has a 16S RNA gene that is at least 95% identical to SEQ ID NO:26.

157. The method of any one of claims 109-118 and 122-156, wherein the Streptococcus intermedius 393 has a 16S RNA gene that is at least 95% identical to SEQ ID NO:27.

158. The method of any one of claims 109-118 and 122-157, wherein the Streptococcus intermedius 393 has a 16S RNA gene that is at least 95% identical to SEQ ID NO:28.

159. The method of any one of claims 109-118 and 122-158, wherein the Streptococcus intermedius 393 has a 16S RNA gene that is at least 95% identical to SEQ ID NO:29.

160. The method of any one of claims 109-118 and 122-159, wherein the Streptococcus intermedius 393 has a 16S RNA gene that is at least 95% identical to SEQ ID NO:30.

161. The method of any one of claims 109-118 and 122-160, wherein the bacterial strain in the composition is viable.

162. The method of any one of claims 109-118 and 122-161, wherein the bacterial strain is lyophilized.

163. The method of any one of claims 109-118 and 122-162, wherein the composition further comprises one or more cryopreservants.

164. The method of any one of claims 109-118 and 122-163, wherein the effective amount of the bacterial strain comprises at least about 1×10³ colony forming units (CFU) of the bacterial strain.

165. The method of any one of claims 109-118 and 122-164, wherein the effective amount of the bacterial strain comprises about 1×10⁴ to about 1×10¹⁵ CFU of the bacterial strain.

166. The method of any one of claims 109-118 and 122-165, wherein the effective amount of the bacterial strain comprises about 1×10⁶ to about 1×10¹⁰ CFU of the bacterial strain.

167. The method of any one of claims 109-118 and 122-160, wherein the bacterial strain in the composition is non-viable.

168. The method of claim 167, wherein the non-viable bacterial strain is heat-killed, irradiated, or lysed.

169. The method of any one of claims 109-118 and 122-168, wherein the method comprises administering the composition to the subject once, twice, or three times per day.

170. The method of any one of claims 109-118 and 122-169, wherein the composition is formulated for topical administration.

171. The method of any one of claims 109-118 and 122-170, wherein the composition is formulated as a paste, gel, cream, spray, mousse, emollient, ointment, foam, or suspension.

172. The method of any one of claims 109-169, wherein the composition is formulated for oral administration.

173. The method of any one of claims 109-169, wherein the composition is formulated for rectal administration.

174. The method of any one of claims 109-173, wherein the composition is formulated as a tablet, a capsule, a powder, or a liquid.

175. The method of any one of claims 109-174, wherein the composition is formulated as a tablet.

176. The method of claim 175, wherein the tablet is coated.

177. The method of claim 176, wherein the coating comprises an enteric coating.

178. The method of any one of claims 109-177, wherein the method further comprises administering another treatment for atopic dermatitis and/or adjunct therapy to the subject.

179. The method of claim 178, wherein the composition comprising the bacterial strain treatment and the treatment for atopic dermatitis and/or adjunct therapy are administered simultaneously.

180. The method of claim 178, wherein the composition comprising the bacterial strain treatment and the treatment for atopic dermatitis and/or adjunct therapy are administered sequentially.

181. The method of claim 178, wherein the composition comprising the bacterial strain further comprises the treatment for atopic dermatitis and/or adjunct therapy.

182. The method of any one of claims 178-181, wherein the treatment for atopic dermatitis and/or adjunct therapy comprises a probiotic.

183. The method of claim 178-182, wherein the treatment for atopic dermatitis and/or adjunct therapy comprises vitamin D, an antihistamine, a corticosteroid, a calcineurin inhibitor, an immunosuppressant, a phosphodiesterase-4 inhibitor, an anti-interleukin-4 receptor (IL-4Rα) antibody, an antibiotic, phototherapy, or a combination thereof.

184. The method of claim 183, wherein the corticosteroid comprises hydrocortisone, clobetasol propionate, betamethasone dipropionate, halobetasol propionate, diflorasone diacetate, fluocinonide, halcinonide, amcinonide, desoximetasone, triamcinolone acetonide, mometasone furoate, fluticasone propionate, betamethasone dipropionate, halometasone, fluocinolone acetonide, hydrocortisone valerate, hydrocortisone butyrate, flurandrenolide, triamcinolone acetonide, mometasone furoate, fluticasone propionate, desonide, fluocinolone acetonide, hydrocortisone valerate, alclometasone dipropionate, triamcinolone acetonide, fluocinolone acetonide, desonide, or a combination thereof.

185. The method of claim 183 or 184, wherein the treatment for atopic dermatitis and/or adjunct therapy comprises diphenhydramine, tacrolimus, pimecrolimus, cyclosporine A, methotrexate, interferon gamma, mycophenolate mofetil, azathioprine, crisaborole, dupilumab, or a combination thereof.

186. The method of any one of claims 109-185, wherein the subject is a human.

187. A method of characterizing a biopsy sample obtained from a subject as comprising a lesion or healthy tissue comprising:
characterizing a biopsy sample that has:
(i) an increased level of one or more bacterial species selected from: Corynebacterium genitalium, Corynebacterium matruchotii, Corynebacterium.striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; and/or
(ii) a decreased level of one or more bacterial species selected from: Brevundimonas nasdae, Capnocytophaga sputigena, Moraxella sp., Neisseria elongata, Staphylococcus felis, Staphylococcus sciuri, and Streptococcus intermedius;
as comprising a lesion; or
(b) characterizing a biopsy sample that does not have:
(i) an increased level of one or more bacterial species selected from: Corynebacterium genitalium, Corynebacterium matruchotii, Corynebacterium.striatum, Corynebacterium tuberculostearicum, Finegoldia magna, Gordonia terrae, Micrococcus luteus, Peptococcus saccharolyticus, Prevotella oris, Staphylococcus aureus, Staphylococcus caprae, Staphylococcus cohnii, Staphylococcus devriesei, Staphylococcus epidermidis, Staphylococcus hominis, Streptococcus mitis, and Turicella otitidis; or
(ii) a decreased level of one or more bacterial species selected from: Brevundimonas nasdae, Capnocytophaga sputigena, Moraxella sp., Neisseria elongata, Staphylococcus felis, Staphylococcus sciuri, and Streptococcus intermedius;
as comprising healthy tissue.


SEQUENCE LISTING (130 sequences)

SEQ ID NO: 1 - DNA, 1426 bp, Brevundimonas nasdae
  misc_feature: Brevundimonas nasdae W1-2B
agagtttgatcatggctcagagcgaacgctggcggcaggcctaacacatgcaagtcgaacgaactcttcggagttagtggcggacgggtgagtaacacgtgggaacgtgcctttaggttcggaataactcagggaaacttgtgctaataccgaatgtgcccttcgggggaaagatttatcgcctttagagcggcccgcgtctgattagctagttggtgaggtaaaggctcaccaaggcgacgatcagtagctggtctgagaggatgatcagccacattgggactgagacacggcccaaactcctacgggaggcagcagtggggaatcttgcgcaatgggcgaaagcctgacgcagccatgccgcgtgaatgatgaaggtcttaggattgtaaaattctttcaccggggacgataatgacggtacccggagaagaagccccggctaacttcgtgccagcagccgcggtaatacgaagggggctagcgttgctcggaattactgggcgtaaagggagcgtaggcggacatttaagtcaggggtgaaatcccggggctcaacctcggaattgcctttgatactgggtgtcttgagtatgagagaggtgtgtggaactccgagtgtagaggtgaaattcgtagatattcggaagaacaccagtggcgaaggcgacacactggctcattactgacgctgaggctcgaaagcgtggggagcaaacaggattagataccctggtagtccacgccgtaaacgatgattgctagttgtcgggatgcatgcatttcggtgacgcagctaacgcattaagcaatccgcctggggagtacggtcgcaagattaaaactcaaaggaattgacgggggcccgcacaagcggtggagcatgtggtttaattcgaagcaacgcgcagaaccttaccaccttttgacatgcctggaccgccagagagatctggctttcccttcggggactaggacacaggtgctgcatggctgtcgtcagctcgtgtcgtgagatgttgggttaagtcccgcaacgagcgcaaccctcgccattagttgccatcatttagttgggaactctaatgggactgccggtgctaagccggaggaaggtggggatgacgtcaagtcctcatggcccttacagggtgggctacacacgtgctacaatggcgactacagagggttaatccttaaaagtcgtctcagttcggattgtcctctgcaactcgagggcatgaagttggaatcgctagtaatcgcggatcagcatgccgcggtgaatacgttcccgggccttgtacacaccgcccgtcacaccatgggagttggttctacccgaaggcgctgcgctgaccgcaaggaaggcaggcgaccacggtagggtcagcgactggggtgaagtcgtaacaaggtaaccgta

SEQ ID NO: 2 - DNA, 1477 bp, Capnocytophaga sputigena
  misc_feature: Capnocytophaga sputigena 4
agagtttgatcctggctcaggatgaacgctagcggcaggcctaacacatgcaagtcgagggagaggttaccttcgggtaaccaaaaccggcgcacgggtgcgtaacgcgtatacaatctgcctttcacatagggatagcccgaagaaatttggattaatacctaatggtatataggagcggcatcgtttttatattaaagctacggtggtgaaagatgagtatgcgttctattagctagttggagaggtaacggctccccaaggcgacgatagataggggttctgagagggatgtcccccacactggtactgagatacggaccagactcctacgggaggcagcagtgaggaatattggacaatggtcggaagactgatccagccatgccgcgtgcaggatgaaggtcctatggattgtaaactgcttttgtaagggaagaataaggagtacgtgtactttgatgacggtaccttacgaataagcatcggctaactccgtgccagcagccgcggtaatacggaggatgcgagcgttatccggaatcattgggtttaaagggtccgtaggcgggctgataagtcagaggtgaaagcgcttagctcaactaagcaactgcctttgaaactgtcagtcttgaatgattgtgaagtagttggaatgtgtagtgtagcggtgaaatgcttagatattacacagaacaccgatagcgaaggcatattactaacaatttattgacgctgatggacgaaagcgtggggagcgaacaggattagataccctggtagtccacgctgtaaacgatggatactagctgtttggagcaatctgagtggctaagcgaaagtgataagtatcccacctggggagtacgcacgcaagtgtgaaactcaaaggaattgacgggggcccgcacaagcggtggagcatgtggtttaattcgatgatacgcgaggaaccttaccaaggtttaaatgggaactgacagaggtagagatacctccttcttcggacagttttcaaggtgctgcatggttgtcgtcagctcgtgccgtgaggtgtcaggttaagtcctataacgagcgcaacccctgccattagttgctaacgagtcaagtcgagccctctagtgggactgccggtgcaaaccgtgaggaaggtggggatgacgtcaaatcatcacggcccttacatcttgggctacacacgtgctacaatggccgttacagagagcagccactgcgcgagcaggagcgaatctataaagacggtcacagttcggatcggagtctgcaactcgactccgtgaagctggaatcgctagtaatcggatatcagccatgatccggtgaatacgttcccgggccttgtacacaccgcccgtcaagccatggaagctgggggtacctgaagacggtcaccgcgaggagctgtttagggtaaaactagtgactggggctaagtcgtaacaaggtaacc

SEQ ID NO: 3 - DNA, 1489 bp, Capnocytophaga sputigena
  misc_feature: Capnocytophaga sputigena 4
agagtttgatcctggctcaggatgaacgctagcggcaggcctaacacatgcaagtcgagggagaggttaccttcgggtaaccaaaaccggcgcacgggtgcgtaacgcgtatacaatctgcctttcacatagggatagcccgaagaaatttggattaatacctaatggtatataggagcggcatcgtttttatattaaagctacggtggtgaaagatgagtatgcgttctattagctagttggagaggtaacggctccccaaggcgacgatagataggggttctgagagggatgtcccccacactggtactgagatacggaccagactcctacgggaggcagcagtgaggaatattggacaatggtcggaagactgatccagccatgccgcgtgcaggatgaaggtcctatggattgtaaactgcttttgtaagggaagaataaggagtacgtgtactttgatgacggtaccttacgaataagcatcggctaactccgtgccagcagccgcggtaatacggaggatgcgagcgttatccggaatcattgggtttaaagggtccgtaggcgggctgataagtcagaggtgaaagcgcttagctcaactaagcaactgcctttgaaactgtcagtcttgaatgattgtgaagtagttggaatgtgtagtgtagcggtgaaatgcttagatattacacagaacaccgatagcgaaggcatattactaacaatttattgacgctgatggacgaaagcgtggggagcgaacaggattagataccctggtagtccatgctgtaaacgatggatactagctgtttggagcaatctgagtggctaagcgaaagtgataagtatcccacctggggagtacgcacgcaagtgtgaaactcaaaggaattgacgggggcccgcacaagcggtggagcatgtggtttaattcgatgatacgcgaggaaccttaccaaggtttaaatgggaactgacagaggtagagatacctccttcttcggacagttttcaaggtgctgcatggttgtcgtcagctcgtgccgtgaggtgtcaggttaagtcctataacgagcgcaacccctgccattagttgctaacgagtcaagtcgagccctctagtgggactgccggtgcaaaccgtgaggaaggtggggatgacgtcaaatcatcacggcccttacatcttgggctacacacgtgctacaatggccgttacagagagcagccactgcgcgagcaggagcgaatctataaagacggtcacagttcggatcggagtctgcaactcgactccgtgaagctggaatcgctagtaatcggatatcaaccatgatccggtgaatacgttcccgggccttgtacacaccgcccgtcaaaccatggaagctgggggtacctgaagacggtcaccgcgaggagctgtttagggtaaaactagtgactggggctaagtcgtaacaaggtagccgtaccggaaagt

SEQ ID NO: 4 - DNA, 1460 bp, Capnocytophaga sputigena
  misc_feature: Capnocytophaga sputigena 4
  misc_feature (55)..(55): n is a, c, g, or t
  misc_feature (256)..(257): n is a, c, g, or t
  misc_feature (334)..(334): n is a, c, g, or t
  misc_feature (519)..(519): n is a, c, g, or t
  misc_feature (1004)..(1006): n is a, c, g, or t
  misc_feature (1071)..(1071): n is a, c, g, or t
atgaagagtttgatcctggctcaggatgaacgctagcggcaggcctaacacatgnaagtcgagggagaggttaccttcgggtaaccaaaaccggcgcacgggtgcgtaacgcgtatacaatctgcctttcacatagggatagcccgaagaaatttggattaatacctaatggtatataggagcggcatcgtttttatattaaagctacggtggtgaaagatgagtatgcgttctattagctagttggagaggtaanngctccccaaggcgacgatagataggggttctgagagggatgtcccccacactggtactgagatacggaccagactcntacgggaggcagcagtgaggaatattggacaatggtcggaagactgatccagccatgccgcgtgcaggatgaaggtcctatggattgtaaactgcttttgtaagggaagaataaggagtacgtgtactttgatgacggtaccttacgaataagcatcggctaactccgtgccagcagccgcggtnatacggaggatgcgagcgttatccggaatcattgggtttaaagggtccgtaggcgggctgataagtcagaggtgaaagcgcttagctcaactaagcaactgcctttgaaactgtcagtcttgaatgattgtgaagtagttggaatgtgtagtgtagcggtgaaatgcttagatattacacagaacaccgatagcgaaggcatattactaacaatttattgacgctgatggacgaaagcgtggggagcgaacaggattagataccctggtagtccatgctgtaaacgatggatactagctgtttggagcaatctgagtggctaagcgaaagtgataagtatcccacctggggagtacgcacgcaagtgtgaaactcaaaggaattgacgggggcccgcacaagcggtggagcatgtggtttaattcgatgatacgcgaggaaccttaccaaggtttaaatgggaactgacagaggtagagataccnnnttcttcggacagttttcaaggtgctgcatggttgtcgtcagctcgtgccgtgaggtgtcaggttnagtcctataacgagcgcaacccctgccattagttgctaacgagtcaagtcgagccctctagtgggactgccggtgcaaaccgtgaggaaggtggggatgacgtcaaatcatcacggcccttacatcttgggctacacacgtgctacaatggccgttacagagagcagccactgcgcgagcaggagcgaatctataaagacggtcacagttcggatcggagtctgcaactcgactccgtgaagctggaatcgctagtaatcggatatcagccatgatccggtgaatacgttcccgggccttgtacacaccgcccgtcaagccatggaagctgggggtacctgaagacggtctccgcaaggagctgtttagggtaaaactagtgactgggg

SEQ ID NO: 5 - DNA, 1515 bp, Capnocytophaga sputigena
  misc_feature: Capnocytophaga sputigena 4
atgaagagtttgatcctggctcaggatgaacgctagcggcaggcctaacacatgcaagtcgagggagaggttaccttcgggtaaccaaaaccggcgcacgggtgcgtaacgcgtatacaatctgcctttcacatagggatagcccgaagaaatttggattaatacctaatggtatataggagcggcatcgtttttatattaaagctacggtggtgaaagatgagtatgcgttctattagctagttggagaggtaacggctccccaaggcgacgatagataggggttctgagagggatgtcccccacactggtactgagatacggaccagactcctacgggaggcagcagtgaggaatattggacaatggtcggaagactgatccagccatgccgcgtgcaggatgaaggtcctatggattgtaaactgcttttgtaagggaagaataaggagtacgtgtactttgatgacggtaccttacgaataagcatcggctaactccgtgccagcagccgcggtaatacggaggatgcgagcgttatccggaatcattgggtttaaagggtccgtaggcgggctgataagtcagaggtgaaagcgcttagctcaactaagcaactgcctttgaaactgtcagtcttgaatgattgtgaagtagttggaatgtgtagtgtagcggtgaaatgcttagatattacacagaacaccgatagcgaaggcatattactaacaatttattgacgctgatggacgaaagcgtggggagcgaacaggattagataccctggtagtccacgctgtaaacgatggatactagctgtttggagcaatctgagtggctaagcgaaagtgataagtatcccacctggggagtacgcacgcaagtgtgaaactcaaaggaattgacgggggcccgcacaagcggtggagcatgtggtttaattcgatgatacgcgaggaaccttaccaaggtttaaatgggaactgacagaggtagagatacctccttcttcggacagttttcaaggtgctgcatggttgtcgtcagctcgtgccgtgaggtgtcaggttaagtcctataacgagcgcaacccctgccattagttgctaacgagtcaagtcgagccctctagtgggactgccggtgcaaaccgtgaggaaggtggggatgacgtcaaatcatcacggcccttacatcttgggctacacacgtgctacaatggccgttacagagagcagccactgcgcgagcaggagcgaatctataaagacggtcacagttcggatcggagtctgcaactcgactccgtgaagctggaatcgctagtaatcggatatcagccatgatccggtgaatacgttcccgggccttgtacacaccgcccgtcaagccatggaagctgggggtacctgaagacggtcaccgcgaggagctgtttagggtaaaactagtgactggggctaagtcgtaacaaggtagccgtaccggaaggtgcggctggaacacctcctttct

SEQ ID NO: 6 - DNA, 1515 bp, Capnocytophaga sputigena; identical to SEQ ID NO: 5
  misc_feature: Capnocytophaga sputigena 4

SEQ ID NO: 7 - DNA, 1505 bp, Moraxella
  misc_feature: Moraxella sp. LMG 5131
  misc_feature (973)..(973): n is a, c, g, or t
attgaacgctggcggcaggcttaacacatgcaagtcgaacgatgactctctagcttgctagagatgattagtggcggacgggtgagtaacatttaggaatctacctagtagtgggggatagctcggggaaactcgaattaataccgcatacgacctacgggtgaaagggggcgcaagctcttgctattagatgagcctaaatcagattagctagttggtggggtaaaggcccaccaaggcgacgatctgtaactggtctgagaggatgatcagtcacaccggaactgagacacggtccggactcctacgggaggcagcagtggggaatattggacaatgggggcaaccctgatccagccatgccgcgtgtgtgaagaaggccttttggttgtaaagcacttttaagcaggaggagaggctaatggttaatacccattagattagacgttacctgcagaataagcaccggctaactctgtgccagcagccgcggtaatacagagggtgcgagcgttaatcggaattactgggcgtaaagcgagtgtaggtggctcattaagtcacatgtgaaatccccgggcttaacctgggaactgcatgtgatactggtggtgctagaatatgtgagagggaagtagaattccaggtgtagcggtgaaatgcgtagagatctggaggaataccgatggcgaaggcagcttcctggcataatattgacactgagattcgaaagcgtgggtagcaaacaggattagataccctggtagtccacgccgtaaacgatgtctactagccgttggggtccttgagactttagtggcgcagttaacgcgataagtagaccgcctggggagtacggccgcaaggttaaaactcaaatgaattgacgggggcccgcacaagcggtggagcatgtggtttaattcgatgcaacgcgaagaaccttacctggtcttgacatagtgagaatctntcagagatgagagagtgccttcgggaactcacatacaggtgctgcatggctgtcgtcagctcgtgtcgtgagatgttgggttaagtcccgcaacgagcgcaacccttttccttatttgccagcgggttaagccgggaactttaaggatactgccagtgacaaactggaggaaggcggggacgacgtcaagtcatcatggcccttacgaccagggctacacacgtgctacaatggtaggtacagagggttgctacacagcgatgtgatgctaatctcaaaaagcctatcgtagtccggattggagtctgcaactcgactccatgaagtcggaatcgctagtaatcgcggatcagaatgccgcggtgaatacgttcccgggccttgtacacaccgcccgtcacaccatgggagtctattgcaccagaagtaggtagcctaacgaaagagggcgcttaccacggtgtggtcgatgactggggtgaagtcgtaacaaggtagccgtaggggaacctgcggctggatcacctcctta

SEQ ID NO: 8 - DNA, 1475 bp, Moraxella
  misc_feature: Moraxella sp. LMG 5131
tggctcaggacgaacgctggcggcgtgcctaatacatgcaagtagaacgctgaagtctggtgcttgcactagacggatgagttgcgaacgggtgagtaacgcgtaggtaacctgcctcatagcgggggataactattggaaacgatagctaataccgcataacagtatttaccgcatggtagatatttgaaaggagcaattgcttcactatgagatggacctgcgttgtattagctagttggtgaggtaacggctcaccaaggcttcgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgtaagagaagaactgtgagaagagtggaaagtttctcacttgacggtatcttaccagaaagggacggctaactacgtgccagcagccgcggtaatacgtaggtcccgagcgttgtccggatttattgggcgtaaagcgagcgcaggcggtttgataagtctgaagtaaaaggctgtggcttaaccatagtacgctttggaaactgtcaaacttgagtgcagaaggggagagtggaattccatgtgtagcggtgaaatgcgtagatatatggaggaacaccggtggcgaaagcggctctctggtctgtaactgacgctgaggctcgaaagcgtggggagcgaacaggattagataccctggtagtccacgccgtaaacgatgagtgctaggtgttgggtcctttccgggactcagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccgatgaccgccctagagatagggtttctcttcggagcatcggtgacaggtggtgcatggttgtcgtcagctcgtgtcgtgagatgttgggttaagtcccgcaacgagcgcaacccctattgttagttgccatcattcagttgggcactctagcgagactgccggtaataaaccggaggaaggtggggatgacgtcaaatcatcatgccccttatgacctgggctacacacgtgctacaatggctggtacaacgagtcgcaagtcggtgacggcaagctaatctcttaaagccagtctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccttttaggagccagccgcctaaggtgggatagatgattgg

SEQ ID NO: 9 - DNA, 1522 bp, Moraxella
  misc_feature: Moraxella sp. LMG 5131
tagagtttgatcatggctcagattgaacgctggcggcaggcttaacacatgcaagtcgaacgatgactctctagcttgctagagatgattagtggcggacgggtgagtaacatttaggaatctacctagtagtgggggatagctcggggaaactcgaattaataccgcatacgacctacgggtgaaagggggcgcaagctcttgctattagatgagcctaaatcagattagctagttggtggggtaaaggcccaccaaggcgacgatctgtaactggtctgagaggatgatcagtcacaccggaactgagacacggtccggactcctacgggaggcagcagtggggaatattggacaatgggggcaaccctgatccagccatgccgcgtgtgtgaagaaggccttttggttgtaaagcactttaagcagggaggagaggctaatggttaatacccattagattagacgttacctgcagaataagcaccggctaactctgtgccagcagccgcggtaatacagagggtgcgagcgttaatcggaattactgggcgtaaagcgagtgtaggtggctcattaagtcacatgtgaaatccccgggcttaacctgggaactgcatgtgatactggtggtgctagaatatgtgagagggaagtagaattccaggtgtagcggtgaaatgcgtagagatctggaggaataccgatggcgaaggcagcttcctggcataatattgacactgagattcgaaagcgtgggtagcaaacaggattagataccctggtagtccacgccgtaaacgatgtctactagccgttggggtccttgagactttagtggcgcagttaacgcgataagtagaccgcctggggagtacggccgcaaggttaaaactcaaatgaattgacgggggcccgcacaagcggtggagcatgtggtttaattcgatgcaacgcgaagaaccttacctggtcttgacatagtgagaatctttcagagatgagagagtgccttcgggaactcacatacaggtgctgcatggctgtcgtcagctcgtgtcgtgagatgttgggttaagtcccgcaacgagcgcaacccttttccttatttgccagcgggttaagccgggaactttaaggatactgccagtgacaaactggaggaaggcggggacgacgtcaagtcatcatggcccttacgaccagggctacacacgtgctacaatggtaggtacagagggttgctacacagcgatgtgatgctaatctcaaaaagcctatcgtagtccggattggagtctgcaactcgactccatgaagtcggaatcgctagtaatcgcggatcagaatgccgcggtgaatacgttcccgggccttgtacacaccgcccgtcacaccatgggagtctattgcaccagaagtaggtagcctaacgaaagagggcgcttaccacggtgtggtcgatgactggggtgaagtcgtaacaaggtagccgtaggggaacctgcggttggatcacctc

SEQ ID NO: 10 - DNA, 1483 bp, Moraxella
  misc_feature: Moraxella sp. LMG 5131
gagtttgatcctggctcagattgaacgctggcggcaggcttaacacatgcaagtcgaacgatgactctctagcttgctagagatgattagtggcggacgggtgagtaacatttaggaatctacctagtagtgggggatagctcggggaaactcgaattaataccgcatacgacctacgggtgaaagggggcgcaagctcttgctattagatgagcctaaatcagattagctagttggtggggtaaaggcccaccaaggcgacgatctgtaactggtctgagaggatgatcagtcacaccggaactgagacacggtccggactcctacgggaggcagcagtggggaatattggacaatgggggcaaccctgatccagccatgccgcgtgtgtgaagaaggccttttggttgtaaagcactttaagcagggaggagaggctaatggttaatacccattagattagacgttacctgcagaataagcaccggctaactctgtgccagcagccgcggtaatacagagggtgcgagcgttaatcggaattactgggcgtaaagcgagtgtaggtggctcattaagtcacatgtgaaatccccgggcttaacctgggaactgcatgtgatactggtggtgctagaatatgtgagagggaagtagaattccaggtgtagcggtgaaatgcgtagagatctggaggaataccgatggcgaaggcagcttcctggcataatattgacactgagattcgaaagcgtgggtagcaaacaggattagataccctggtagtccacgccgtaaacgatgtctactagccgttggggtccttgagactttagtggcgcagttaacgcgataagtagaccgcctggggagtacggccgcaaggttaaaactcaaatgaattgacgggggcccgcacaagcggtggagcatgtggtttaattcgatgcaacgcgaagaaccttacctggtcttgacatagtgagaatctytcagagatgagagagtgccttcgggaactcacatacaggtgctgcatggctgtcgtcagctcgtgtcgtgagatgttgggttaagtcccgcaacgagcgcaacccttttccttatttgccagcgggttaagccgggaactttaaggatactgccagtgacaaactggaggaaggcggggacgacgtcaagtcatcatggcccttacgaccagggctacacacgtgctacaatggtaggtacagagggttgctacacagcgatgtgatgctaatctcaaaaagcctatcgtagtccggattggagtctgcaactcgactccatgaagtcggaatcgctagtaatcgcggatcagaatgccgcggtgaatacgttcccgggccttgtacacaccgcccgtcacaccatgggagtctattgcaccagaagtaggtagcctaacgaaagagggcgcttaccacggtgtggtcgatgactggggtgaagtcgtaac

SEQ ID NO: 11 - DNA, 1492 bp, Moraxella
  misc_feature: Moraxella sp. LMG 5131
gagtttgatcctggctcagattgaacgctggcggcaggcttaacacatgcaagtcgaacgatgactctctagcttgctagagatgattagtggcggacgggtgagtaacatttaggaatctacctagtagtgggggatagctcggggaaactcgaattaataccgcatacgacctacgggtgaaagggggcgcaagctcttgctattagatgagcctaaatcagattagctagttggtggggtaaaggcccaccaaggcgacgatctgtaactggtctgagaggatgatcagtcacaccggaactgagacacggtccggactcctacgggaggcagcagtggggaatattggacaatgggggcaaccctgatccagccatgccgcgtgtgtgaagaaggccttttggttgtaaagcactttaagcagggaggagaggctaatggttaatacccattagattagacgttacctgcagaataagcaccggctaactctgtgccagcagccgcggtaatacagagggtgcgagcgttaatcggaattactgggcgtaaagcgagtgtaggtggctcattaagtcacatgtgaaatccccgggcttaacctgggaactgcatgtgatactggtggtgctagaatatgtgagagggaagtagaattccaggtgtagcggtgaaatgcgtagagatctggaggaataccgatggcgaaggcagcttcctggcataatattgacactgagattcgaaagcgtgggtagcaaacaggattagataccctggtagtccacgccgtaaacgatgtctactagccgttggggtccttgagactttagtggcgcagttaacgcgataagtagaccgcctggggagtacggccgcaaggttaaaactcaaatgaattgacgggggcccgcacaagcggtggagcatgtggtttaattcgatgcaacgcgaagaaccttacctggtcttgacatagtgagaatctytcagagatgagagagtgccttcgggaactcacatacaggtgctgcatggctgtcgtcagctcgtgtcgtgagatgttgggttaagtcccgcaacgagcgcaacccttttccttatttgccagcgggttaagccgggaactttaaggatactgccagtgacaaactggaggaaggcggggacgacgtcaagtcatcatggcccttacgaccagggctacacacgtgctacaatggtaggtacagagggttgctacacagcgatgtgatgctaatctcaaaaagcctatcgtagtccggattggagtctgcaactcgactccatgaagtcggaatcgctagtaatcgcggatcagaatgccgcggtgaatacgttcccgggccttgtacacaccgcccgtcacaccatgggagtctattgcaccagaagtaggtagcctaacgaaagagggcgcttaccacggtgtggtcgatgactggggtgaagtcgtaacaaggtagcc

SEQ ID NO: 12 - DNA, 1551 bp, Neisseria elongata
  misc_feature: Neisseria elongata ATCC 29315
agagattgaacataagagtttgatcctggctcagattgaacgctggcggcatgctttacacatgcaagtcggacggcagcggggtagtgcttgcactactgccggcgagtggcgaacgggtgagtaatatattggaacgtaccgagtaatgggggataaccaatcgaaagattggctaataccgcatacgctctgaggaggaaagcaggggaccttcgggccttgcgttattcgagcggccaatatctgattagctagttggtggggtaaaggcctaccaaggcgacgatcagtagcgggtctgagaggatgatccgccacactgggactgagacacggcccagactcctacgggaggcagcagtggggaattttggacaatgggcgcaagcctgatccagccatgccgcgtgtctgaagaaggccttcgggttgtaaaggacttttgttagggaagaaaaaggaacggttaatacccgtttctgctgacggtacctaaagaataagcaccggctaactacgtgccagcagccgcggtaatacgtagggtgcgagcgttaatcggaattactgggcgtaaagcgagcgcagacggttacttaagcaggatgtgaaatccccgggctcaacctgggaactgcgttctgaactgggtagctagagtatgtcagaggggggtagaattccacgtgtagcagtgaaatgcgtagagatgtggaggaataccgatggcgaaggcagccccctgggataatactgacgttcatgctcgaaagcgtgggtagcaaacaggattagataccctggtagtccacgccctaaacgatgtcgattagctgttgggcagcttgactgcttagtagcgtagctaacgcgtgaaatcgaccgcctggggagtacggtcgcaagattaaaactcaaaggaattgacggggacccgcacaagcggtggatgatgtggattaattcgatgcaacgcgaagaaccttacctggtcttgacatgtacggaaccttccagagacggaagggtgccttcgggagccgtaacacaggtgctgcatggctgtcgtcagctcgtgtcgtgagatgttgggttaagtcccgcaacgagcgcaacccttgtcattagttgccatcatttagttgggcactctaatgagactgccggtgacaaaccggaggaaggtggggatgacgtcaagtcctcatggcccttatgaccagggcttcacacgtcatacaatggtcggtacaaagggtagccaagccgcgaggcggagccaatctcataaaaccgatcgtagtccggattgcactctgcaactcgagtgcatgaagtcggaatcgctagtaatcgcaggtcagcatactgcggtgaatacgttcccgggtcttgtacacaccgcccgtcacaccatgggagtgggggataccagaagtaggtagggtaaccgtaaggagcccgcttaccacggtatgcttcatgactggggtgaagtcgtaacaaggtagccgtaggggaacctgcggctggatcacctcctttcta

SEQ ID NO: 13 - DNA, 1551 bp, Neisseria elongata; identical to SEQ ID NO: 12
  misc_feature: Neisseria elongata ATCC 29315

SEQ ID NO: 14 - DNA, 1551 bp, Neisseria elongata
  misc_feature: Neisseria elongata ATCC 29315
agagattgaacataagagtttgatcctggctcagattgaacgctggcggcatgctttacacatgcaagtcggacggcagcggggtagtgcttgcactactgccggcgagtggcgaacgggtgagtaatatattggaacgtaccgagtaatgggggataaccaatcgaaagattggctaataccgcatacgctctgaggaggaaagcaggggaccttcgggccttgcgttattcgagcggccaatatctgattagctagttggtggggtaaaggcctaccaaggcgacgatcagtagcgggtctgagaggatgatccgccacactgggactgagacacggcccagactcctacgggaggcagcagtggggaattttggacaatgggcgcaagcctgatccagccatgccgcgtgtctgaagaaggccttcgggttgtaaaggacttttgttagggaagaaaaaggaacggttaatacccgtttctgctgacggtacctaaagaataagcaccggctaactacgtgccagcagccgcggtaatacgtagggtgcgagcgttaatcggaattactgggcgtaaagcgagcgcagacggttacttaagcaggatgtgaaatccccgggctcaacctgggaactgcgttctgaactgggtagctagagtatgtcagaggggggtagaattccacgtgtagcagtgaaatgcgtagagatgtggaggaataccgatggcgaaggcagccccctgggataatactgacgttcatgctcgaaagcgtgggtagcaaacaggattagataccctggtagtccacgccctaaacgatgtcgattagctgttgggtagcttgactgcttagtagcgtagctaacgcgtgaaatcgaccgcctggggagtacggtcgcaagattaaaactcaaaggaattgacggggacccgcacaagcggtggatgatgtggattaattcgatgcaacgcgaagaaccttacctggtcttgacatgtacggaaccttccagagacggaagggtgccttcgggagccgtaacacaggtgctgcatggctgtcgtcagctcgtgtcgtgagatgttgggttaagtcccgcaacgagcgcaacccttgtcattagttgccatcatttagttgggcactctaatgagactgccggtgacaaaccggaggaaggtggggatgacgtcaagtcctcatggcccttatgaccagggcttcacacgtcatacaatggtcggtacaaagggtagccaagccgcgaggcggagccaatctcataaaaccgatcgtagtccggattgcactctgcaactcgagtgcatgaagtcggaatcgctagtaatcgcaggtcagcatactgcggtgaatacgttcccgggtcttgtacacaccgcccgtcacaccatgggagtgggggataccagaagtaggtagggtaaccgtaaggagcccgcttaccacggtatgcttcatgactggggtgaagtcgtaacaaggtagccgtaggggaacctgcggctggatcacctcctttcta

SEQ ID NO: 15 - DNA, 1551 bp, Neisseria elongata; identical to SEQ ID NO: 12
  misc_feature: Neisseria elongata ATCC 29315

SEQ ID NO: 16 - DNA, 1544 bp, Neisseria elongata
  misc_feature: Neisseria elongata ATCC 29315
ttgaacataagagtttgatcctggctcagattgaacgctggcggcatgctttacacatgcaagtcggacggcagcggggtagtgcttgcactactgccggcgagtggcgaacgggtgagtaatatattggaacgtaccgagtaatgggggataaccaatcgaaagattggctaataccgcatacgctctgaggaggaaagcaggggaccttcgggccttgcgttattcgagcggccaatatctgattagctagttggtggggtaaaggcctaccaaggcgacgatcagtagcgggtctgagaggatgatccgccacactgggactgagacacggcccagactcctacgggaggcagcagtggggaattttggacaatgggcgcaagcctgatccagccatgccgcgtgtctgaagaaggccttcgggttgtaaaggacttttgttagggaagaaaaggaacggttaatacccgtttctgctgacggtacctaaagaataagcaccggctaactacgtgccagcagccgcggtaatacgtagggtgcgagcgttaatcggaattactgggcgtaaagcgagcgcagacggttacttaagcaggatgtgaaatccccgggctcaacctgggaactgcgttctgaactgggtagctagagtatgtcagaggggggtagaattccacgtgtagcagtgaaatgcgtagagatgtggaggaataccgatggcgaaggcagccccctgggataatactgacgttcatgctcgaaagcgtgggtagcaaacaggattagataccctggtagtccacgccctaaacgatgtcgattagctgttgggcagcttgactgcttagtagcgtagctaacgcgtgaaatcgaccgcctggggagtacggtcgcaagattaaaactcaaaggaattgacggggacccgcacaagcggtggatgatgtggattaattcgatgcaacgcgaagaaccttacctggtcttgacatgtacggaaccttccagagacggaagggtgccttcgggagccgtaacacaggtgctgcatggctgtcgtcagctcgtgtcgtgagatgttgggttaagtcccgcaacgagcgcaacccttgtcattagttgccatcatttagttgggcactctaatgagactgccggtgacaaaccggaggaaggtggggatgacgtcaagtcctcatggcccttatgaccagggcttcacacgtcatacaatggtcggtacaaagggtagccaagccgcgaggcggagccaatctcataaaaccgatcgtagtccggattgcactctgcaactcgagtgcatgaagtcggaatcgctagtaatcgcaggtcagcatactgcggtgaatacgttcccgggtcttgtacacaccgcccgtcacaccatgggagtgggggataccagaagtaggtagggtaaccgtaaggagcccgcttaccacggtatgcttcatgactggggtgaagtcgtaacaaggtagccgtaggggaacctgcggctggatcacctcctttct

SEQ ID NO: 17 - DNA, 1544 bp, Neisseria elongata; identical to SEQ ID NO: 16
  misc_feature: Neisseria elongata ATCC 29315

SEQ ID NO: 18 - DNA, 1475 bp, Staphylococcus felis
  misc_feature: Staphylococcus felis GD521
aggatgaacgctggcggcgtgcctaatacatgcaagtcgagcgaactgaagaggagcttgctcctttgacgttagcggcggacgggtgagtaacacgtgggtaacctacctataagactggaataactccgggaaaccggggctaatgccggataatatgttgaaccgcatggttcaacagtgaaagacggttttgctgtcacttatagatggacccgcgccgcattagctagttggtgaggtaacggctcaccaaggcgacgatgcgtagccgacctgagagggtgatcggccacactggaactgagacacggtccagactcctacgggaggcagcagtagggaatcttccgcaatggacgaaagtctgacggagcaacgccgcgtgagtgatgaaggtcttcggatcgtaaagctctgttgttagggaagaacaaatgtgtaagtaactgtgcacatcttgacggtacctaaccagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcggctttctggtctgcaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacggggacccgcacaagcggtggagcatgtggtttaattcgaagcaacgcgaagaaccttaccaaatcttgacatcctttgaccgctctagagatagagttttcctcttcggaggacaaagtgacaggtggtgcatggttgtcgtcagctcgtgtcgtgagatgttgggttaagtcccgcaacgagcgcaacccttaagcttagttgccatcattaagttgggcactctaggctgactgccggtgacaaaccggaggaaggtggggatgacgtcaaatcatcatgccccttatgatttgggctacacacgtgctacaatggacaatacaaagggcagcgaaaccgtgaggtcaagccaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccatttggagctagccgtcgaaggtgggacaaatgattggggtg

SEQ ID NO: 19 - DNA, 1528 bp, Staphylococcus sciuri
  misc_feature: Staphylococcus sciuri SC116
gatgaacgctggcggcgtgcctaatacatgcaagtcgagcgaacagatgagaagcttgcttctctgatgttagcggcggacgggtgagtaacacgtgggtaacctacctataagactgggataactccgggaaaccggggctaataccggataatattttgaaccgcatggttcaatagtgaaagacggtttcggctgtcacttatagatggacccgcgccgtattagctagttggtaaggtaacggcttaccaaggcgacgatacgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttgttagggaagaacaaatttgttagtaactgaacaagtcttgacggtacctaaccagaaagccacggctaactacgtgccagcagccgcggtaatacgtaggtggcaagcgttatccggaattattgggcgtaaagcgcgcgtaggcggtttcttaagtctgatgtgaaagcccacggctcaaccgtggagggtcattggaaactgggaaacttgagtgcagaagaggagagtggaattccatgtgtagcggtgaaatgcgcagagatatggaggaacaccagtggcgaaggcggctctctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgaccgctctagagatagagtcttccccttcgggggacaaagtgacaggtggtgcatggttgtcgtcagctcgtgtcgtgagatgttgggttaagtcccgcaacgagcgcaacccttaagcttagttgccatcattaagttgggcactctaggttgactgccggtgacaaaccggaggaaggtggggatgacgtcaaatcatcatgccccttatgatttgggctacacacgtgctacaatggataatacaaagggcagcgaatccgcgaggccaagcaaatcccataaaattattctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttaggagctagccgtcgaaggtgggacaaatgattggggtgaagtcgtaacaaggtagccgtatcggaaggtgcggctggatcacctcctttct

SEQ ID NO: 20 - DNA, 1366 bp, Streptococcus intermedius
  misc_feature: Streptococcus intermedius 393
gagttgcgaacgggtgagtaacgcgtaggtaacctgcctggtagcgggggataactattggaaacgatagctaataccgcataagaacatttactgcatggtagatgtttaaaaggtgcaaatgcatcactaccagatggacctgcgttgtattagctagtaggtgaggtaacggctcacctaggcgacgatacatagccgacctgagagggtgatcggccacactgggactgagacacggcccagactcctacgggaggcagcagtagggaatcttcggcaatggggggaaccctgaccgagcaacgccgcgtgagtgaagaaggttttcggatcgtaaagctctgttgttaaggaagaacgagtgtgagaatggaaagttcatactgtgacggtacttaaccagaaagggacggctaactacgtgccagcagccgcggtaatacgtaggtcccgagcgttgtccggatttattgggcgtaaagcgagcgcaggcggttagataagtctgaagttaaaggcagtggctcaaccattgtaggctttggaaactgtttaacttgagtgcagaaggggagagtggaattccatgtgtagcggtgaaatgcgtagatatatggaggaacaccggtggcgaaagcggctctctggtctgtaactgacgctgaggctcgaaagcgtggggagcgaacaggattagataccctggtagtccacgccgtaaacgatgagtgctaggtgttaggtcctttccgggacttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccgatgcccgctctagagatagagctttacttcggtacatcggtgacaggtggtgcatggttgtcgtcagctcgtgtcgtgagatgttgggttaagtcccgcaacgagcgcaacccttattgttagttgccatcattcagttgggcactctagcgagactgccggtaataaaccggaggaaggtggggatgacgtcaaatcatcatgccccttatgacctgggctacacacgtgctacaatggctggtacaacgagtcgcaagccggtgacggcaagctaatctctgaaagccagtctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccgtaaggag

SEQ ID NO: 21 - DNA, 1560 bp, Streptococcus intermedius
  misc_feature: Streptococcus intermedius 393
ctataaatgagagtttgatcctggctcaggacgaacgctggcggcgtgcctaatacatgcaagtagaacgcacaggatgcaccgtagtttactacaccgtattctgtgagttgcgaacgggtgagtaacgcgtaggtaacctgcctggtagcgggggataactattggaaacgatagctaataccgcataagaacatttactgcatggtagatgtttaaaaggtgcaaatgcatcactaccagatggacctgcgttgtattagctagtaggtgaggtaacggctcacctaggcgacgatacatagccgacctgagagggtgatcggccacactgggactgagacacggcccagactcctacgggaggcagcagtagggaatcttcggcaatggggggaaccctgaccgagcaacgccgcgtgagtgaagaaggttttcggatcgtaaagctctgttgttaaggaagaacgagtgtgagaatggaaagttcatactgtgacggtacttaaccagaaagggacggctaactacgtgccagcagccgcggtaatacgtaggtcccgagcgttgtccggatttattgggcgtaaagcgagcgcaggcggttagataagtctgaagttaaaggcagtggctcaaccattgtaggctttggaaactgtttaacttgagtgcagaaggggagagtggaattccatgtgtagcggtgaaatgcgtagatatatggaggaacaccggtggcgaaagcggctctctggtctgtaactgacgctgaggctcgaaagcgtggggagcgaacaggattagataccctggtagtccacgccgtaaacgatgagtgctaggtgttaggtcctttccgggacttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccgatgcccgctctagagatagagctttacttcggtacatcggtgacaggtggtgcatggttgtcgtcagctcgtgtcgtgagatgttgggttaagtcccgcaacgagcgcaacccttattgttagttgccatcattcagttgggcactctagcgagactgccggtaataaaccggaggaaggtggggatgacgtcaaatcatcatgccccttatgacctgggctacacacgtgctacaatggctggtacaacgagtcgcaagccggtgacggcaagctaatctctgaaagccagtctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccgtaaggagccagccgcctaaggtgggatagatgattggggtgaagtcgtaacaaggtagccgtatcggaaggtgcggctggatcacctcctttct

SEQ ID NO: 22 - DNA, 1561 bp, Streptococcus intermedius
  misc_feature: Streptococcus intermedius 393
aatgagagtttgatcctggctcaggacgaacgctggcggcgtgcctaatacatgcaagtagaacgcacaggatgcaccgtagtttactacaccgtattctgtgagttgcgaacgggtgagtaacgcgtaggtaacctgcctggtagcgggggataactattggaaacgatagctaataccgcataagaacatttactgcatggtagatgtttaaaaggtgcaaatgcatcactaccagatggacctgcgttgtattagctagtaggtgaggtaacggctcacctaggcgacgatacatagccgacctgagagggtgatcggccacactgggactgagacacggcccagactcctacgggaggcagcagtagggaatcttcggcaatggggggaaccctgaccgagcaacgccgcgtgagtgaagaaggttttcggatcgtaaagctctgttgttaaggaagaacgagtgtgagaatggaaagttcatactgtgacggtacttaaccagaaagggacggctaactacgtgccagcagccgcggtaatacgtaggtcccgagcgttgtccggatttattgggcgtaaagcgagcgcaggcggttagataagtctgaagttaaaggcagtggctcaaccattgtaggctttggaaactgtttaacttgagtgcagaaggggagagtggaattccatgtgtagcggtgaaatgcgtagatatatggaggaacaccggtggcgaaagcggctctctggtctgtaactgacgctgaggctcgaaagcgtggggagcgaacaggattagataccctggtagtccacgccgtaaacgatgagtgctaggtgttaggtcctttccgggacttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccgatgcccgctctagagatagagctttacttcggtacatcggtgacaggtggtgcatggttgtcgtcagctcgtgtcgtgagatgttgggttaagtcccgcaacgagcgcaacccttattgttagttgccatcattcagttgggcactctagcgagactgccggtaataaaccggaggaaggtggggatgacgtcaaatcatcatgccccttatgacctgggctacacacgtgctacaatggctggtacaacgagtcgcaagccggtgacggcaagctaatctctgaaagccagtctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccgtaaggagccagccgcctaaggtgggatagatgattggggtgaagtcgtaacaaggtagccgtatcggaaggtgcggctggatcacctcctttctaaggaa

SEQ ID NO: 23 - DNA, 1561 bp, Streptococcus intermedius; identical to SEQ ID NO: 22
  misc_feature: Streptococcus intermedius 393

SEQ ID NO: 24 - DNA, 1561 bp, Streptococcus intermedius; identical to SEQ ID NO: 22
  misc_feature: Streptococcus intermedius 393

SEQ ID NO: 25 - DNA, 1561 bp, Streptococcus intermedius; identical to SEQ ID NO: 22
  misc_feature: Streptococcus intermedius 393

SEQ ID NO: 26 - DNA, 1561 bp, Streptococcus intermedius; identical to SEQ ID NO: 22
  misc_feature: Streptococcus intermedius 393

SEQ ID NO: 27 - DNA, 1560 bp, Streptococcus intermedius
  misc_feature: Streptococcus intermedius 393
atgagagtttgatcctggctcaggacgaacgctggcggcgtgcctaatacatgcaagtaggacgcacagtttataccgtagcttgctacaccatagactgtgagttgcgaacgggtgagtaacgcgtaggtaacctgcctattagagggggataactattggaaacgatagctaataccgcataacagtatgtaacacatgttagatgcttgaaagatgcaattgcatcgctagtagatggacctgcgttgtattagctagtaggtagggtaatggcctacctaggcgacgatacatagccgacctgagagggtgatcggccacactgggactgagacacggcccagactcctacgggaggcagcagtagggaatcttcggcaatggggggaaccctgaccgagcaacgccgcgtgagtgaagaaggttttcggatcgtaaagctctgttgttaaggaagaacgagtgtgagaatggaaagttcatactgtgacggtacttaaccagaaagggacggctaactacgtgccagcagccgcggtaatacgtaggtcccgagcgttgtccggatttattgggcgtaaagcgagcgcaggcggttagaaaagtctgaagtgaaaggcagtggctcaaccattgtaggctttggaaactgtttaacttgagtgcagaaggggagagtggaattccatgtgtagcggtgaaatgcgtagatatatggaggaacaccggtggcgaaagcggctctctggtctgtaactgacgctgaggctcgaaagcgtggggagcgaacaggattagataccctggtagtccacgccgtaaacgatgagtgctaggtgttgggtcctttccgggactcagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccgatgctatttctagagataggaagtttcttcggaacatcggtgacaggtggtgcatggttgtcgtcagctcgtgtcgtgagatgttgggttaagtcccgcaacgagcgcaacccttattgttagttgccatcattaagttgggcactctagcgagactgccggtaataaaccggaggaaggtggggatgacgtcaaatcatcatgccccttatgacctgggctacacacgtgctacaatggctggtacaacgagtcgcaagccggtgacggcaagctaatctctgaaagccagtctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccgtaaggagccagccgcctaaggtgggatagatgattggggtgaagtcgtaacaaggtagccgtatcggaaggtgcggctggatcacctcctttctaaggaa

SEQ ID NO: 28 - DNA, 1544 bp, Streptococcus intermedius
  misc_feature: Streptococcus intermedius 393
tttgatcctggttcaggacgaacgctggcggcgtgcctaatacatgcaagtagaacgcacaggatgcaccgtagtttactacaccgtattctgtgagttgcgaacgggtgagtaacgcgtaggtaacctgcctggtagcgggggataactattggaaacgatagctaataccgcataagaacatttactgcatggtagatgtttaaaaggtgcaaatgcatcactaccagatggacctgcgttgtattagctagtaggtgaggtaacggctcacctaggcgacgatacatagccgacctgagagggtgatcggccacactgggactgagacacggcccagactcctacgggaggcagcagtagggaatcttcggcaatggggggaaccctgaccgagcaacgccgcgtgagtgaagaaggttttcggatcgtaaagctctgttgttaaggaagaacgagtgtgagaatggaaagttcatactgtgacggtacttaaccagaaagggacggctaactacgtgccagcagccgcggtaatacgtaggtcccgagcgttgtccggatttattgggcgtaaagcgagcgcaggcggttagataagtctgaagttaaaggcagtggctcaaccattgtaggctttggaaactgtttaacttgagtgcagaaggggagagtggaattccatgtgtagcggtgaaatgcgtagatatatggaggaacaccggtggcgaaagcggctctctggtctgtaactgacgctgaggctcgaaagcgtggggagcgaacaggattagataccctggtagtccacgccgtaaacgatgagtgctaggtgttaggtcctttccgggacttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccgatgcccgctctagagatagagctttacttcggtacatcggtgacaggtggtgcatggttgtcgtcagctcgtgtcgtgagatgttgggttaagtcccgcaacgagcgcaacccttattgttagttgccatcattcagttgggcactctagcgagactgccggtaataaaccggaggaaggtggggatgacgtcaaatcatcatgccccttatgacctgggctacacacgtgctacaatggctggtacaacgagtcgcaagccggtgacggcaagctaatctctgaaagccagtctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccgtaaggagccagccgcctaaggtgggatagatgattggggtgaagtcgtaacaaggtagccgtatcggaaggtgcggctggatcacctcctt

SEQ ID NO: 29 - DNA, 1526 bp, Streptococcus intermedius
  misc_feature: Streptococcus intermedius 393
  misc_feature (8)..(8): n is a, c, g, or t
tttgatcntggttcaggacgaacgctggcggcgtgcctaatacatgcaagtagaacgcacaggatgcaccgtagtttactacaccgtattctgtgagttgcgaacgggtgagtaacgcgtaggtaacctgcctggtagcgggggataactattggaaacgatagctaataccgcataagaacatttactgcatggtagatgtttaaaaggtgcaaatgcatcactaccagatggacctgcgttgtattagctagttggtgaggtaacggctcaccaaggcaacgatacatagccgacctgagagggtgatcggccacactgggactgagacacggcccagactcctacgggaggcagcagtagggaatcttcggcaatggggggaaccctgaccgagcaacgccgcgtgagtgaagaaggttttcggatcgtaaagctctgttgttaaggaagaacgtgtgtgagagtggaaagttcacacagtgacggtacttaaccagaaagggacggctaactacgtgccagcagccgcggtaatacgtaggtcccgagcgttgtccggatttattgggcgtaaagcgagcgcaggcggttagataagtctgaagtgaaaggcagtggctcaaccattgtaggctttggaaactgtttaacttgagtgcagaaggggagagtggaattccatgtgtagcggtgaaatgcgtagatatatggaggaacaccggtggcgaaagcggctctctggtctgtaactgacgctgaggctcgaaagcgtggggagcgaacaggattagataccctggtagtccacgccgtaaacgatgagtgctaggtgttaggtcctttccgggacttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccctctgaccactctagagatagagtttcccttcggggcagaggtgacaggtggtgcatggttgtcgtcagctcgtgtcgtgagatgttgggttaagtcccgcaacgagcgcaacccttattgttagttgccatcattcagttgggcactctagcgagactgccggtaataaaccggaggaaggtggggatgacgtcaaatcatcatgccccttatgacctgggctacacacgtgctacaatggctggtacaacgagtcgcaagccggtgacggcaagctaatctctgaaagccagtctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccgtaaggagccagccgcctaaggtgggatagatgattggggtgaagtcgtaacaaggtagccgtatcggaaggtg

SEQ ID NO: 30 - DNA, 1560 bp, Streptococcus intermedius; identical to SEQ ID NO: 21
  misc_feature: Streptococcus intermedius 393

SEQ ID NO: 31 - DNA, 1443 bp, Corynebacterium genitalium
  misc_feature: Corynebacterium genitalium 418H
agagtttgatcctggctcaggacgaacgctggcggcgtgcttaacacatgcaagtcgaacggaaaggccctttgcttgcaaggggtactcgagtggcgaacgggtgagtaacacgtgggtgatctgccttgcacttcgggataagcctgggaaactgggtctaataccggataggaccatgctttagtgtgtgtggtggaaagtttttcggtgcaagatgagcccgcggcctatcagcttgttggtggggtaatggcctaccaaggcgacgacggtagccgcctgagaggtgtacgcacattggactgagacacggccagactctacggaggcagcagtgggatattgcacaatggcgcaagctgatgcagcgacgcgcgtgggatgacgctcggttgtaactccttcgctaggaagaagacatgtgtggtgacggtacctgataagaagcaccggctaactacgtgccagcagcgcggtaatacgtaggtgcgacgttgtccggaattactggcgtaaagagctcgtaggtggtttgtcgcgtcgtctgtgaaatcccggggcttaacttcggcgtgcaggcgatacggcataactagagtgctgtaggggagactggaattcctggtgtagcggtgaaatgcgcagatatcaggaggaacaccgatgcgaaggcaggtctctgggcagttactgacgctgaggacgaaagcatgggtagcgaacaggattagataccctggtagtccatgccgtaaacggtgggcgctaggtgtgggggttttatttacgattcccgtgccgtagctaacgcgttaagcccccgcctggggagtacggccgcaagtaaaactcaaaggaattgacgggggcccgcacaagcgcggagcatgtggattaattcgatgcaacgcgaagaaccttacctgggcttgacatacaccggatcgctgcagagatgtagtttcccttgtggctggtgtacaggtggtgcatggttgtcgtcagctcgtgtcgtgagatgttgggttaagtccgcaacgagcgcaaccttgtcttgtgttgccagcacgttatggtgggactcgcgagagactgccgggttaactcggaggaaggtgggatgacgtcaaatcatcatgccccttatgtccagggcttcacacatgctacaatggtcggtacagtgggttgcgataccgtgaggtggagctaatcccttaaagccggtctcagttcggattggagtctgcaactcgactccatgaagtcggagtcgctactaatcgcagatcagcaacgctgcggtgaatacgttcccgggccttgtacacaccgcccgtcacctcatgaaagttggtaacacccgaagccagtggcccaaacttgttagggagctgtcgaaggtgggattggcgattgggacgaagtcgtaacaaggta

SEQ ID NO: 32 - DNA, 1560 bp, Corynebacterium matruchotii
  misc_feature: Corynebacterium matruchotii ATCC 33806
tttttttggagagtttgatcctggctcaggacgaacgctggcggcgtgcttaacacatgcaagtcgaacggaaaggcccccaccttggtgggggtgctcgagtggcgaacgggtgagtaacacgtgggtgatctgccctgtacttcgggataagcttgggaaactgggtctaataccggataggaccatggtttagtgtctcatggtggaaaaattattgttttggtgcaggatgagctcgcggcctatcagcttgttggtggggtaatggcctaccaaggcgtcgacgggtagccggcctgagagggtggacggccacattgggactgagatacggcccagactcctacgggaggcagcagtggggaatattgcacaatgggcgcaagcctgatgcagcgacgccgcgtgggggatgacggccttcgggttgtaaactcctttcgttagggacgaagccacaccttttttgggtgtggtgacggtacctttgttaagaagcaccggctaactacgtgccagcagccgcggtaatacgtagggtgcgagcgttgtccggaattactgggcgtaaagagctcgtaggtggtttgttgcgtcgtctgtgaaattccggggcttaacttcggggtggcaggcgatacgggcataactagagtgctgtaggggagactggaattcctggtgtagcggtggaatgcgcagatatcaggaggaacaccgatggcgaaggcaggtctctgggcagtaactgacgctgaggagcgaaagcatggggagcgaacaggattagataccctggtagtccatgctgtaaacggtgggcgttaggtgtggggaccggtttggtttctgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatgcaccagacggttgtagagatacgtacctccctttgtggttggtgtgcaggtggtgcatggttgtcgtcagctcgtgtcgtgagatgttgggttaagtcccgcaacgagcgcaacccttgtcttatgttgccagcactttgggtggggactcatgagagactgccggggttaactcggaggaaggtggggatgacgtcaaatcatcatgccctttatgcccagggcttcacacatgctacaatggttagtacaacgcgcggcaaacctgtgagggtgagcgaatcgctgaaagctaatctcagttcggattggggtctgcaactcgaccccatgaagtcggagtcgctagtaatcgcagatcagcaatgctgcggtgaatacgttcccgggccttgtacacaccgcccgtcacgtcatgaaagttggtaacacccgaagcccatggcctaaccaccgtttggtggggggagtggtcgaaggtgggattggcgattgggacgaagtcgtaacaaggtagccgtaccggaaggtgcggctggatcacctcctttctaaggaga

SEQ ID NO: 33 - DNA, 1553 bp, Corynebacterium matruchotii
  misc_feature: Corynebacterium matruchotii ATCC 33806
tttttttggagagtttgatcctggctcaggacgaacgctggcggcgtgcttaacacatgcaagtcgaacggaaaggcccccaccttggtgggggtgctcgagtggcgaacgggtgagtaacacgtgggtgatctgccctgtacttcgggataagcttgggaaactgggtctaataccggataggaccatggtttagtgtctcatggtggaaaaattattgttttggtgcaggatgagctcgcggcctatcagcttgttggtggggtaatggcctaccaaggcgtcgacgggtagccggcctgagagggtggacggccacattgggactgagatacggcccagactcctacgggaggcagcagtggggaatattgcacaatgggcgcaagcctgatgcagcgacgccgcgtgggggatgacggccttcgggttgtaaactcctttcgttagggacgaagccacaccttttttgggtgtggtgacggtacctttgttaagaagcaccggctaactacgtgccagcagccgcggtaatacgtagggtgcgagcgttgtccggaattactgggcgtaaagagctcgtaggtggtttgttgcgtcgtctgtgaaattccggggcttaacttcggggtggcaggcgatacgggcataactagagtgctgtaggggagactggaattcctggtgtagcggtggaatgcgcagatatcaggaggaacaccgatggcgaaggcaggtctctgggcagtaactgacgctgaggagcgaaagcatggggagcgaacaggattagataccctggtagtccatgctgtaaacggtgggcgttaggtgtggggaccggtttggtttctgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatgcaccagacggttgtagagatacgtacctccctttgtggttggtgtgcaggtggtgcatggttgtcgtcagctcgtgtcgtgagatgttgggttaagtcccgcaacgagcgcaacccttgtcttatgttgccagcactttgggtggggactcatgagagactgccggggttaactcggaggaaggtggggatgacgtcaaatcatcatgccctttatgcccagggcttcacacatgctacaatggttagtacaacgcgcggcaaacctgtgagggtgagcgaatcgctgaaagctaatctcagttcggattggggtctgcaactcgaccccatgaagtcggagtcgctagtaatcgcagatcagcaatgctgcggtgaatacgttcccgggccttgtacacaccgcccgtcacgtcatgaaagttggtaacacccgaagcccatggcctaaccaccgtttggtggggggagtggtcgaaggtgggattggcgattgggacgaagtcgtaacaaggtagccgtaccggaaggtgcggctggatcacctcctttct

SEQ ID NO: 34 - DNA, 1553 bp, Corynebacterium matruchotii; identical to SEQ ID NO: 33
  misc_feature: Corynebacterium matruchotii ATCC 33806

SEQ ID NO: 35 - DNA, 1477 bp, Corynebacterium striatum
  misc_feature: Corynebacterium striatum ATCC 6940
  misc_feature (39)..(39): n is a, c, g, or t
ctggctcaggacgaacgctggcggcgtgcttaacacatncaagtcgaacggaaaggcctgcagcttgctgcgggtactcgagtggcgaacgggtgagtaacacgtgggtgatctgccctgcacttcgggataagcttgggaaactgggtctaataccagataggaccactgtttagtgtcagttgtggaaagttttttcggtgcaagatgagctcgcggcctatcagcttgttggtggggtaatggcctaccaaggcgtcgacgggtagccggcctgagagggtgtacggccacattgggactgagatacggcccaactcctacgggaggcagcagtggggaatattgcacaatgggcggaagcctgatgcagcgacgccgcgtgggggatgaaggccttcgggttgtaaactcctttcgacagggacgaagcgtaagtgacggtacctgtataagaagcaccggctaactacgtgccagcagccgcggtaatacgtagggtgcgagcgttgtccggaattactgggcgtaaagagctcgtaggtggtttgtcgcgtcgtctgtgaaattccggggcttaactccgggcgtgcaggcgatacgggctgacttgagtgctgtaggggagactggaattcctggtgtagcggtgaaatgcgcagatatcaggaggaacaccgatggcgaaggcaggtctctgggcagtaactgacgctgaggagcgaaagcatgggtagcgaacaggattagataccctggtagtccatgccgtaaacggtgggcgctaggtgtagggggcttccacgtcttctgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggcttgacatatacaagacaggcgtagagatacgtcctcccttgtggtttgtatacaggtggtgcatggttgtcgtcagctcgtgtcgtgagatgttgggttaagtcccgcaacgagcgcaacccttgtcttatgttgccagcatttggttggggactcatgagagactgccggggtcaactcggaggaaggtggggatgacgtcaaatcatcatgccccttatgtccagggcttcacacatgctacaatggtcggtacaacgcgcagcgacactgtgaggtggagcgaatcgctgaaagccggccttagttcggattggggtctgcaactcgaccccatgaagtcggagtcgctagtaatcgcagatcagcaatgctgcggtgaatacgttcccgggccttgtacacaccgcccgtcacgtcatgaaagttggtaacacccgaagccagtggctcaaactcgttagggagctgtcgaaggtgggatcggcgattgggacgaagtcgtaacaaggtagccgtaccggaag

SEQ ID NO: 36 - DNA, 1530 bp, Corynebacterium striatum
  misc_feature: Corynebacterium striatum ATCC 6940
tttttttgtggagagtttgatcctggctcaggacgaacgctggcggcgtgcttaacacatgcaagtcgaacggaaaggcctgcagcttgctgcgggtactcgagtggcgaacgggtgagtaacacgtgggtgatctgccttgcacttcgggataagcttgggaaactgggtctaataccggataggacagctgtttagtgtcagttgtggaaagttttttcggtgcaagatgagctcgcggcctatcagcttgttggtggggtaatggcctaccaaggcgtcgacgggtagccggcctgagagggtgtacggccacattgggactgagatacggcccagactcctacgggaggcagcagtggggaatattgcacaatgggcggaagcctgatgcagcgacgccgcgtgggggatgaaggccttcgggttgtaaactcctttcgacagggacgaagcgtaagtgacggtacctgtataagaagcaccggctaactacgtgccagcagccgcggtaatacgtagggtgcgagcgttgtccggaattactgggcgtaaagagctcgtaggtggtttgtcgcgtcgtctgtgaaattccggggcttaactccgggcgtgcaggcgatacgggctgacttgagtgctgtaggggagactggaattcctggtgtagcggtgaaatgcgcagatatcaggaggaacaccgatggcgaaggcaggtctctgggcagtaactgacgctgaggagcgaaagcatgggtagcgaacaggattagataccctggtagtccatgccgtaaacggtgggcgctaggtgtagggggcttccacgtcttctgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggcttgacatatacaagacaggcgtagagatacgtcctcccttgtggtttgtatacaggtggtgcatggttgtcgtcagctcgtgtcgtgagatgttgggttaagtcccgcaacgagcgcaacccttgtcttatgttgccagcatttggttggggactcatgagagactgccggggtcaactcggaggaaggtggggatgacgtcaaatcatcatgccccttatgtccagggcttcacacatgctacaatggtcggtacaacgcgcagcgacactgtgaggtggagcgaatcgctgaaagccggccttagttcggattggggtctgcaactcgaccccatgaagtcggagtcgctagtaatcgcagatcagcaacgctgcggtgaatacgttcccgggccttgtacacaccgcccgtcacgtcatgaaagttggtaacacccgaagccagtggctcaaactcgttagggagctgtcgaaggtgggatcggcgattgggacgaagtcgtaacaaggtagccgtaccggaaggtgcggctggatcacctcctttctaaggag

SEQ ID NO: 37 - DNA, 1522 bp, Corynebacterium striatum
  misc_feature: Corynebacterium striatum ATCC 6940
tttttgtggagagtttgatcctggctcaggacgaacgctggcggcgtgcttaacacatgcaagtcgaacggaaaggcctgcagcttgctgcgggtactcgagtggcgaacgggtgagtaacacgtgggtgatctgccttgcacttcgggataagcttgggaaactgggtctaataccggataggacagctgtttagtgtcagttgtggaaagttttttcggtgcaagatgagctcgcggcctatcagcttgttggtggggtaatggcctaccaaggcgtcgacgggtagccggcctgagagggtgtacggccacattgggactgagatacggcccagactcctacgggaggcagcagtggggaatattgcacaatgggcggaagcctgatgcagcgacgccgcgtgggggatgaaggccttcgggttgtaaactcctttcgacagggacgaagcgtaagtgacggtacctgtataagaagcaccggctaactacgtgccagcagccgcggtaatacgtagggtgcgagcgttgtccggaattactgggcgtaaagagctcgtaggtggtttgtcgcgtcgtctgtgaaattccggggcttaactccgggcgtgcaggcgatacgggctgacttgagtgctgtaggggagactggaattcctggtgtagcggtgaaatgcgcagatatcaggaggaacaccgatggcgaaggcaggtctctgggcagtaactgacgctgaggagcgaaagcatgggtagcgaacaggattagataccctggtagtccatgccgtaaacggtgggcgctaggtgtagggggcttccacgtcttctgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggcttgacatatacaagacaggcgtagagatacgtcctcccttgtggtttgtatacaggtggtgcatggttgtcgtcagctcgtgtcgtgagatgttgggttaagtcccgcaacgagcgcaacccttgtcttatgttgccagcatttggttggggactcatgagagactgccggggtcaactcggaggaaggtggggatgacgtcaaatcatcatgccccttatgtccagggcttcacacatgctacaatggtcggtacaacgcgcagcgacactgtgaggtggagcgaatcgctgaaagccggccttagttcggattggggtctgcaactcgaccccatgaagtcggagtcgctagtaatcgcagatcagcaacgctgcggtgaatacgttcccgggccttgtacacaccgcccgtcacgtcatgaaagttggtaacacccgaagccagtggctcaaactcgttagggagctgtcgaaggtgggatcggcgattgggacgaagtcgtaacaaggtagccgtaccggaaggtgcggctggatcacctcctttct

SEQ ID NO: 38 - DNA, 1522 bp, Corynebacterium striatum; identical to SEQ ID NO: 37
  misc_feature: Corynebacterium striatum ATCC 6940

SEQ ID NO: 39 - DNA, 1510 bp, Corynebacterium tuberculostearicum
  misc_feature: Corynebacterium tuberculostearicum CIP 102857
agagtttgatcctggctcaggatgaacgctggcggcgtgcttaacacatgcaagtcgaacggaaaggccctgcttgcagggtactcgagtggcgaacgggtgagtaacacgtgggtgatctgccctgcacttcgggataagcctgggaaactgggtctaataccggataggagccatttttagtgtgatggttggaaagttttttcggtgtaggatgagctcgcggcctatcagcttgttggtggggtaatggcctaccaaggcggcgacgggtagccggcctgagagggtggacggccacattgggactgagatacggcccagactcctacgggaggcagcagtggggaatattgcacaatgggcgcaagcctgatgcagcgacgccgcgtgggggatgacggccttcgggttgtaaactcctttcgctagggacgaagctttttgtgacggtacctagataagaagcaccggctaactacgtgccagcagccgcggtaatacgtagggtgcgagcgttgtccggaattactgggcgtaaagggctcgtaggtggtttgtcgcgtcgtctgtgaaattccggggcttaactccgggcgtgcaggcgatacgggcataacttgagtactgtaggggtaactggaattcctggtgtagcggtgaaatgcgcagatatcaggaggaacaccgatggcgaaggcaggttactgggcagttactgacgctgaggagcgaaagcatgggtagcgaacaggattagataccctggtagtccatgccgtaaacggtgggcgctaggtgtgagggtcttttcacgactttcgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggcttgacatacactggatcgggctagagatagtctttccctttgtggctggtgtacaggtggtgcatggttgtcgtcagctcgtgtcgtgagatgttgggttaagtcccgcaacgagcgcaacccttgtcttatgttgccagcatttggttggggactcatgagagactgccggggtcaactcggaggaaggtggggatgacgtcaaatcatcatgccccttatgtccagggcttcacacatgctacaatggtcggtacaacgcgcagcgacactgtgaggtggagcgaatcgctgaaagccggccttagttcggattggggtctgcaactcgaccccatgaagtcggagtcgctagtaatcgcagatcagcaatgctgcggtgaatacgttcccgggccttgtacacaccgcccgtcacgtcatgaaagttggtaacacccgaagccggtggcccaaacttgttagggagccgtcgaaggtgggatcggcgattgggacgaagtcgtaacaaggtacccgtaccggaaggtgcggctggatcacctcctt

SEQ ID NO: 40 - DNA, 1527 bp, Finegoldia magna
  misc_feature: Finegoldia magna 312
attaaattgagagtttgatcctggctcaggacgaacgctggcggcgtgcttaacacatgcaagtcgaacgggatttagtaaacagaagcttcggtggaagattactaatgagagtggcgaacgggtgagtaacgcgtgagcaacctgcctatgacagtgggatagcctcgggaaaccgggattaataccgcataaaatcgtagaaacacatgttttaacggtcaaagatttatcggtcatagatgggctcgcgtctgattagctagttggtgagataacagcccaccaaggcgacgatcagtagccggtctgagaggatgaacggccacattggaactgagacacggtccaaactcctacgggaggcagcagtggggaatattgcacaatgggggaaaccctgatgcagcgacgccgcgtgaacgaagaaggtattcgtatcgtaaagttctgtcctatgggaagataatgacagtaccatagaagaaagctccggctaaatacgtgccagcagccgcggtaatacgtatggagcgagcgttgtccggaattattgggcgtaaagggtacgcaggcggtttaataagtcgaatgttaaagatcggggctcaaccccgtaaagcattggaaactgataaacttgagtagtggagaggaaagtggaattcctagtgtagtggtgaaatacgtagatattaggaggaataccagtagcgaaggcgactttctggacacaaactgacgctgaggtacgaaagcgtggggagcaaacaggattagataccctggtagtccacgccgtaaacgatgaatgctaggtgttgggggtcaaacctcggtgccgaagttaacacattaagcattccgcctggggagtacgcacgcaagtgtgaaactcaaaggaattgacggggacccgcacaagcagcggagcatgtggtttaattcgatgcaacgcgaagaaccttaccagggcttgacatgtgggtgaaaggtatagagatatacccctctctttatgagacatccatacaggtggtgcatggttgtcgtcagctcgtgtcgtgagatgttgggttaagtcccgcaacgagcgcaacccctatacttagttaccagcgagtaatgtcggggactctaagtagactgccgatgacaaatcggaggaaggtggggatgacgtcaaatcatcatgccctttatgtcctgggctacacacgtgctacaatggttggtacagagggaagctatatagtgatataatgcaaaactccaaagccaatcccagttcggattgtaggctgcaactcgcctacatgaagtcggagttgctagtaatcgcggatcagaatgtcgcggtgaatgcgttcccgggtcttgtacacaccgcccgtcacaccatgggagttgataatacccgaagcctgtgacctaattgaggagcagtcgaaggtaggattgatgactggggtgaagtcgtaacaaggtagccgtatcggaaggtgcggctggatcacctcctttcta

SEQ ID NO: 41 - DNA, 1412 bp, Finegoldia magna
  misc_feature: Finegoldia magna 312
gagtggcgaacgggtgagtaacgcgtgagcaacctgcctatgacagtgggatagcctcgggaaaccgggattaataccgcataaaatcgtagaaacacatgttttaacggtcaaagatttatcggtcatagatgggctcgcgtctgattagctagttggtgagataacagcccaccaaggcgacgatcagtagccggtctgagaggatgaacggccacattggaactgagacacggtccaaactcctacgggaggcagcagtggggaatattgcacaatgggggaaaccctgatgcagcgacgccgcgtgaacgaagaaggtattcgtatcgtaaagttctgtcctatgggaagataatgacagtaccatagaagaaagctccggctaaatacgtgccagcagccgcggtaatacgtatggagcgagcgttgtccggaattattgggcgtaaagggtacgcaggcggtttaataagtcgaatgttaaagatcggggctcaaccccgtaaagcattggaaactgataaacttgagtagtggagaggaaagtggaattcctagtgtagtggtgaaatacgtagatattaggaggaataccagtagcgaaggcgactttctggacacaaactgacgctgaggtacgaaagcgtggggagcaaacaggattagataccctggtagtccacgccgtaaacgatgaatgctaggtgttgggtgtcaaagctcggtgccgaagttaacacattaagcattccgcctggggagtacgcacgcaagtgtgaaactcaaaggaattgacggggacccgcacaagcagcggagcatgtggtttaattcgatgcaacgcgaagaaccttaccagggcttgacatgtgggtgaaaggtatagagatatacccctctctttatgagacatccatacaggtggtgcatggttgtcgtcagctcgtgtcgtgagatgttgggttaagtcccgcaacgagcgcaacccctatacttagttaccagcgagtaatgtcggggactctaagtagactgccgatgacaaatcggaggaaggtggggatgacgtcaaatcatcatgccctttatgtcctgggctacacacgtgctacaatggttggtacagagggaagctatatagtgatataatgcaaaactccaaagccaatcccagttcggattgtaggctgcaactcgcctacatgaagtcggagttgctagtaatcgcggatcagaatgtcgcggtgaatgcgttcccgggtcttgtacacaccgcccgtcacaccatgggagttgataatacccgaagcctgtgacctaattgaggagcagtcgaaggtaggattgatgactggggtgaagtcgtaacaaggtagccgtatcggaaggtgcggctggatcacctcctt

SEQ ID NO: 42 - DNA, 1525 bp, Finegoldia magna
  misc_feature: Finegoldia magna 312
ttaaattgagagtttgatcctggctcaggacgaacgctggcggcgtgcttaacacatgcaagtcgaacgggatttagtaaacagaagcttcggtggaagattactaatgagagtggcgaacgggtgagtaacgcgtgagcaacctgcctatgacagtgggatagcctcgggaaaccgggattaataccgcataaaatcgtagaaacacatgttttaacggtcaaagatttatcggtcatagatgggctcgcgtctgattagctagttggtgagataacagcccaccaaggcgacgatcagtagccggtctgagaggatgaacggccacattggaactgagacacggtccaaactcctacgggaggcagcagtggggaatattgcacaatgggggaaaccctgatgcagcgacgccgcgtgaacgaagaaggtattcgtatcgtaaagttctgtcctatgggaagataatgacagtaccatagaagaaagctccggctaaatacgtgccagcagccgcggtaatacgtatggagcgagcgttgtccggaattattgggcgtaaagggtacgcaggcggtttaataagtcgaatgttaaagatcggggctcaaccccgtaaagcattggaaactgataaacttgagtagtggagaggaaagtggaattcctagtgtagtggtgaaatacgtagatattaggaggaataccagtagcgaaggcgactttctggacacaaactgacgctgaggtacgaaagcgtggggagcaaacaggattagataccctggtagtccacgccgtaaacgatgaatgctaggtgttgggggtcaaacctcggtgccgaagttaacacattaagcattccgcctggggagtacgcacgcaagtgtgaaactcaaaggaattgacggggacccgcacaagcagcggagcatgtggtttaattcgatgcaacgcgaagaaccttaccagggcttgacatgtgggtgaaaggtatagagatatacccctctctttatgagacatccatacaggtggtgcatggttgtcgtcagctcgtgtcgtgagatgttgggttaagtcccgcaacgagcgcaacccctatacttagttaccagcgagtaatgtcggggactctaagtagactgccgatgacaaatcggaggaaggtggggatgacgtcaaatcatcatgccctttatgtcctgggctacacacgtgctacaatggttggtacagagggaagctatatagtgatataatgcaaaactccaaagccaatcccagttcggattgtaggctgcaactcgcctacatgaagtcggagttgctagtaatcgcggatcagaatgtcgcggtgaatgcgttcccgggtcttgtacacaccgcccgtcacaccatgggagttgataatacccgaagcctgtgacctaattgaggagcagtcgaaggtaggattgatgactggggtgaagtcgtaacaaggtagccgtatcggaaggtgcggctggatcacctcctttct

SEQ ID NO: 43 - DNA, 1526 bp, Finegoldia magna
  misc_feature: Finegoldia magna 312
attaaattgagagtttgatcctggctcaggacgaacgctggcggcgtgcttaacacatgcaagtcgaacgggatttagtaaacagaagcttcggtggaagattactaatgagagtggcgaacgggtgagtaacgcgtgagcaacctgcctatgacagtgggatagcctcgggaaaccgggattaataccgcataaaatcgtagaaacacatgttttaacggtcaaagatttatcggtcatagatgggctcgcgtctgattagctagttggtgagataacagcccaccaaggcgacgatcagtagccggtctgagaggatgaacggccacattggaactgagacacggtccaaactcctacgggaggcagcagtggggaatattgcacaatgggggaaaccctgatgcagcgacgccgcgtgaacgaagaaggtattcgtatcgtaaagttctgtcctatgggaagataatgacagtaccatagaagaaagctccggctaaatacgtgccagcagccgcggtaatacgtatggagcgagcgttgtccggaattattgggcgtaaagggtacgcaggcggtttaataagtcgaatgttaaagatcggggctcaaccccgtaaagcattggaaactgataaacttgagtagtggagaggaaagtggaattcctagtgtagtggtgaaatacgtagatattaggaggaataccagtagcgaaggcgactttctggacacaaactgacgctgaggtacgaaagcgtggggagcaaacaggattagataccctggtagtccacgccgtaaacgatgaatgctaggtgttgggggtcaaacctcggtgccgaagttaacacattaagcattccgcctggggagtacgcacgcaagtgtgaaactcaaaggaattgacggggacccgcacaagcagcggagcatgtggtttaattcgatgcaacgcgaagaaccttaccagggcttgacatgtgggtgaaaggtatagagatatacccctctctttatgagacatccatacaggtggtgcatggttgtcgtcagctcgtgtcgtgagatgttgggttaagtcccgcaacgagcgcaacccctatacttagttaccagcgagtaatgtcggggactctaagtagactgccgatgacaaatcggaggaaggtggggatgacgtcaaatcatcatgccctttatgtcctgggctacacacgtgctacaatggttggtacagagggaagctatatagtgatataatgcaaaactccaaagccaatcccagttcggattgtaggctgcaactcgcctacatgaagtcggagttgctagtaatcgcggatcagaatgtcgcggtgaatgcgttcccgggtcttgtacacaccgcccgtcacaccatgggagttgataatacccgaagcctgtgacctaattgaggagcagtcgaaggtaggattgatgactggggtgaagtcgtaacaaggtagccgtatcggaaggtgcggctggatcacctcctttct

SEQ ID NO: 44 - DNA, 1481 bp, Gordonia terrae
  misc_feature: Gordonia terrae T5
ggacgaacgctggcggcgtgcttaacacatgcaagtcgaacggaaaggcccagcttgctgggtactcgagtggcgaacgggtgagtaacacgtgggtgatctgccctgcactctgggataagcctgggaaactgggtctaataccggatatgaccaactgtcgcatggtggttggtggaaagcttttgcggtgtgggatgggcccgcgccctatcagcttgttggtggggtaatggcctaccaaggcgacgacgggtagccgacctgagagggtgatcggccacactgggactgagacacggcccagactcctacgggaggcagcagtggggaatattgcacaatgggcgcaagcctgatgcagcgacgccgcgtgagggatgacggccttcgggttgtaaacctctttcaccagggacgaagcgtgagtgacggtacctggagaagaagcaccggccaactacgtgccagcagccgcggtaatacgtagggtgcgagcgttgtccggaattactgggcgtaaagagctcgtaggcggtttgtcgcgtcgtctgtgaaattctgcaactcaattgcaggcgtgcaggcgatacgggcagacttgagtactacaggggagactggaattcctggtgtagcggtgaaatgcgcagatatcaggaggaacaccggtggcgaaggcgggtctctgggtagtaactgacgctgaggagcgaaagcgtgggtagcgaacaggattagataccctggtagtccacgccgtaaacggtgggtactaggtgtgggttccttttcacgggatccgtgccgtagctaacgcattaagtaccccgcctggggagtacggccgcaaggctaaaactcaaaggaattgacgggggcccgcacaagcggcggagcatgtggattaattcgatgcaacgcgaagaaccttacctgggtttgacatacaccagacgcggctagagatagtcgttcccttgtggttggtgtacaggtggtgcatggctgtcgtcagctcgtgtcgtgagatgttgggttaagtcccgcaacgagcgcaacccttgtcctgtattgccagcgggttatgccggggacttgcaggagactgccggggtcaactcggaggaaggtggggatgacgtcaagtcatcatgccccttatgtccagggcttcacacatgctacaatggctggtacagagggctgcgataccgtgaggtggagcgaatcccttaaagccagtctcagttcggattggggtctgcaactcgaccccatgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagctgtcgaaggtgggatcggcgattgggacgaagtcgtaacaaggtagccgtaccggaaggtgcgg

SEQ ID NO: 45 - DNA, 1527 bp, Gordonia terrae
  misc_feature: Gordonia terrae T5
ttttcatggagagtttgatcctggctcaggacgaacgctggcggcgtgcttaacacatgcaagtcgaacggaaaggcccagcttgctgggtactcgagtggcgaacgggtgagtaacacgtgggtgatctgccctgcactctgggataagcctgggaaactgggtctaataccggatatgaccaactgtcgcatggtggttggtggaaagcttttgcggtgtgggatgggcccgcggcctatcagcttgttggtggggtaatggcctaccaaggcgacgacgggtagccgacctgagagggtgatcggccacactgggactgagacacggcccagactcctacgggaggcagcagtggggaatattgcacaatgggcgcaagcctgatgcagcgacgccgcgtgagggatgacggccttcgggttgtaaacctctttcaccagggacgaagcgtgagtgacggtacctggagaagaagcaccggccaactacgtgccagcagccgcggtaatacgtagggtgcgagcgttgtccggaattactgggcgtaaagagctcgtaggcggtttgtcgcgtcgtctgtgaaattctgcaactcaattgcaggcgtgcaggcgatacgggcagacttgagtactacaggggagactggaattcctggtgtagcggtgaaatgcgcagatatcaggaggaacaccggtggcgaaggcgggtctctgggtagtaactgacgctgaggagcgaaagcgtgggtagcgaacaggattagataccctggtagtccacgccgtaaacggtgggtactaggtgtgggttccttttcacgggatccgtgccgtagctaacgcattaagtaccccgcctggggagtacggccgcaaggctaaaactcaaaggaattgacgggggcccgcacaagcggcggagcatgtggattaattcgatgcaacgcgaagaaccttacctgggtttgacatacaccagacgcggctagagatagtcgttcccttgtggttggtgtacaggtggtgcatggctgtcgtcagctcgtgtcgtgagatgttgggttaagtcccgcaacgagcgcaacccttgtcctgtattgccagcgggttatgccggggacttgcaggagactgccggggtcaactcggaggaaggtggggatgacgtcaagtcatcatgccccttatgtccagggcttcacacatgctacaatggctggtacagagggctgcgataccgtgaggtggagcgaatcccttaaagccagtctcagttcggattggggtctgcaactcgaccccatgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagctgtcgaaggtgggatcggcgattgggacgaagtcgtaacaaggtagccgtaccggaaggtgcggctggatcacctcctttct

SEQ ID NO: 46 - DNA, 1531 bp, Gordonia terrae
  misc_feature: Gordonia terrae T5
ttttttcatggagagtttgatcctggctcaggacgaacgctggcggcgtgcttaacacatgcaagtcgaacggaaaggcccagcttgctgggtactcgagtggcgaacgggtgagtaacacgtgggtgatctgccctgcactctgggataagcctgggaaactgggtctaataccggatatgaccaactgtcgcatggtggttggtggaaagcttttgcggtgtgggatgggcccgcggcctatcagcttgttggtggggtaatggcctaccaaggcgacgacgggtagccgacctgagagggtgatcggccacactgggactgagacacggcccagactcctacgggaggcagcagtggggaatattgcacaatgggcgcaagcctgatgcagcgacgccgcgtgagggatgacggccttcgggttgtaaacctctttcaccagggacgaagcgtgagtgacggtacctggagaagaagcaccggccaactacgtgccagcagccgcggtaatacgtagggtgcgagcgttgtccggaattactgggcgtaaagagctcgtaggcggtttgtcgcgtcgtctgtgaaattctgcaactcaattgcaggcgtgcaggcgatacgggcagacttgagtactacaggggagactggaattcctggtgtagcggtgaaatgcgcagatatcaggaggaacaccggtggcgaaggcgggtctctgggtagtaactgacgctgaggagcgaaagcgtgggtagcgaacaggattagataccctggtagtccacgccgtaaacggtgggtactaggtgtgggttccttttcacgggatccgtgccgtagctaacgcattaagtaccccgcctggggagtacggccgcaaggctaaaactcaaaggaattgacgggggcccgcacaagcggcggagcatgtggattaattcgatgcaacgcgaagaaccttacctgggtttgacatacaccagacgcggctagagatagtcgttcccttgtggttggtgtacaggtggtgcatggctgtcgtcagctcgtgtcgtgagatgttgggttaagtcccgcaacgagcgcaacccttgtcctgtattgccagcgggttatgccggggacttgcaggagactgccggggtcaactcggaggaaggtggggatgacgtcaagtcatcatgccccttatgtccagggcttcacacatgctacaatggctggtacagagggctgcgataccgtgaggtggagcgaatcccttaaagccagtctcagttcggattggggtctgcaactcgaccccatgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagctgtcgaaggtgggatcggcgattgggacgaagtcgtaacaaggtagccgtaccggaaggtgcggctggatcacctcctttctaa

SEQ ID NO: 47 - DNA, 1531 bp, Gordonia terrae; identical to SEQ ID NO: 46
  misc_feature: Gordonia terrae T5

SEQ ID NO: 48 - DNA, 1531 bp, Micrococcus luteus
  misc_feature: Micrococcus luteus SK58
ttttacggagagtttgatcctggctcaggatgaacgctggcggcgtgcttaacacatgcaagtcgaacgatgaagcccagcttgctgggtggattagtggcgaacgggtgagtaacacgtgagtaacctgcccttaactctgggataagcctgggaaactgggtctaataccggataggagcgcctaccgcatggtgggtgttggaaagatttatcggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagtagggaagaagcgaaagtgacggtacctgcagaagaagcaccggctaactacgtgccagcagccgcggtaatacgtagggtgcgagcgttatccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtggggaccattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttctcgatcgccgtagagatacggtttcccctttggggcgggttcacaggtggtgcatggttgtcgtcagctcgtgtcgtgagatgttgggttaagtcccgcaacgagcgcaaccctcgttccatgttgccagcacgtaatggtggggactcatgggagactgccggggtcaactcggaggaaggtgaggacgacgtcaaatcatcatgccccttatgtcttgggcttcacgcatgctacaatggccggtacaatgggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagtcggtaacacccgaagccggtggcctaacccttgtggggggagccgtcgaaggtgggaccagcgattgggactaagtcgtaacaaggtagccgtaccggaaggtgcggctggatcacctcctttctaagga

SEQ ID NO: 49 - DNA, 1527 bp, Micrococcus luteus
  misc_feature: Micrococcus luteus SK58
tttttacggagagtttgatcctggctcaggatgaacgctggcggcgtgcttaacacatgcaagtcgaacgatgaagcccagcttgctgggtggattagtggcgaacgggtgagtaacacgtgagtaacctgcccttaactctgggataagcctgggaaactgggtctaataccggataggagcgcctaccgcatggtgggtgttggaaagatttatcggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagtagggaagaagcgaaagtgacggtacctgcagaagaagcaccggctaactacgtgccagcagccgcggtaatacgtagggtgcgagcgttatccggaattattgggcgtaaagagctcgtaggcggtttgtcgcgtctgtcgtgaaagtccggggcttaaccccggacctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtggggaccattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttctcgatcgccgtagagatacggtttcccctttggggcgggttcacaggtggtgcatggttgtcgtcagctcgtgtcgtgagatgttgggttaagtcccgcaacgagcgcaaccctcgttccatgttgccagcacgtaatggtggggactcatgggagactgccggggtcaactcggaggaaggtgaggacgacgtcaaatcatcatgccccttatgtcttgggcttcacgcatgctacaatggccggtacaatgggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagtcggtaacacccgaagccggtggcctaacccttgtggggggagccgtcgaaggtgggaccagcgattgggactaagtcgtaacaaggtagccgtaccggaaggtgcggctggatcacctcctttct

SEQ ID NO: 50 - DNA, 1527 bp, Micrococcus luteus
  misc_feature: Micrococcus luteus SK58
tttttacggagagtttgatcctggctcaggatgaacgctggcggcgtgcttaacacatgcaagtcgaacgatgaagcccagcttgctgggtggattagtggcgaacgggtgagtaacacgtgagtaacctgcccttaactctgggataagcctgggaaactgggtctaataccggataggagcgcctaccgcatggtgggtgttggaaagatttatcggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagtagggaagaagcgaaagtgacggtacctgcagaagaagcaccggctaactacgtgccagcagccgcggtaatacgtagggtgcgagcgttatccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtggggaccattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttctcgatcgccgtagagatacggtttcccctttggggcgggttcacaggtggtgcatggttgtcgtcagctcgtgtcgtgagatgttgggttaagtcccgcaacgagcgcaaccctcgttccatgttgccagcacgtaatggtggggactcatgggagactgccggggtcaactcggaggaaggtgaggacgacgtcaaatcatcatgccccttatgtcttgggcttcacgcatgctacaatggccggtacaatgggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagtcggtaacacccgaagccggtggcctaacccttgtggggggagccgtcgaaggtgggaccagcgattgggactaagtcgtaacaaggtagccgtaccggaaggtgcggctggatcacctcctttct

SEQ ID NO: 51 - DNA, 1527 bp, Micrococcus luteus; identical to SEQ ID NO: 49
  misc_feature: Micrococcus luteus SK58

SEQ ID NO: 52 - DNA, 1512 bp, Peptococcus saccharolyticus
  misc_feature: Peptococcus saccharolyticus S1
agagtttgatcctggctcaggatgaacgctggcggcgtgcctaatacatgcaagtcgagcgaacagacgaggagcttgctcctctgacgttagcggcggacgggtgagtaacacgtggataacctacctataagactgggataacttcgggaaaccggagctaataccagataacatgttgaaccgcatggttcaacagtgaaagacggtcttgctgtcacttatagatggatccgcgccgcattagctagttggtaaggtaacggcttaccaaggcaacgatgcgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaatgtgtaagtaactatgcacgtcttgacggcacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgacccctctagagatagagttttccccttcgggggacagagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaaccccataaagttgttctcagttcggattgtagtctgcaactcgactatatgaagctggaatcgctagtaatcgtagatcagcatgctacggtgaatacgttcccgggtcttgtacacaccgcccgtcacaccacgagagtttgtaacacccgaagccggtggagtaaccttttggagctagccgtcgaaggtgggacaaatgattggggtgaagtcgtaacaaggtaacc

SEQ ID NO: 53 - DNA, 1498 bp, Peptococcus saccharolyticus
  misc_feature: Peptococcus saccharolyticus S1
  misc_feature (22)..(22): n is a, c, g, or t
ctggctcaggatgtaygctggnggcgtgcctaatacatgcaagtcgagcgaacagacgaggagcttgctcctctgacgttagcggcggacgggtgagtaacacgtggataacctacctataagactgggataacttcgggaaaccggagctaataccagataacatgttgaaccgcatggttcaacagtgaaagacggtcttgctgtcacttatagatggatccgcgccgcattagctagttggtaaggtaacggcttaccaaggcaacgatgcgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaatgtgtaagtaactatgcacgtcttgacggcacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgacccctctagagatagagttttccccttcgggggacagagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaaccccataaagttgttctcagttcggattgtagtctgcaactcgactatatgaagctggaatcgctagtaatcgtagatcagcatgctacggtgaatacgttcccgggtcttgtacacaccgcccgtcacaccacgagagtttgtaacacccgaagccggtggagtaacctkttggagctagccgtcgaaggtgggacaaatgattggggtgaagtcgtaacaaggta

SEQ ID NO: 54 - DNA, 1537 bp, Prevotella oris
  misc_feature: Prevotella oris F0302
ttacaatggagagtttgatcctggctcaggatgaacgctggctacaggcttaacacatgcaagtcgaggggaaacgacggggaagcttgcttccttcgggcgtcgaccggcgcacgggtgagtaacgcgtatccaacctgcctctgactgagggataacccgtcgaaagtcggcctaatacctcatggcatcgtctgcgggcatccaacgacgattaaagatttcatcggtcagggatggggatgcgtctgattagcttgttggcggggtaacggcccaccaaggctacgatcagtaggggttctgagaggaaggtcccccacattggaactgagacacggtccaaactcctacgggaggcagcagtgaggaatattggtcaatgggcgagagcctgaaccagccaagtagcgtgcaggaagacggccctatgggttgtaaactgcttttatgcggggataaagtgagggacgtgtccttcattgcaggtaccgcatgaataaggaccggctaattccgtgccagcagccgcggtaatacggaaggtccgggcgttatccggatttattgggtttaaagggagcgtaggccgtggattaagcgtgttgtgaaatgcaggtgctcaacgtctgcactgcagcgcgaactggtccacttgagtgtgcgcaacgcaggcggaattcgtcgtgtagcggtgaaatgcttagatatgacgaagaactccgattgcgaaggcagcttgcgggagcacaactgacgctgaagctcgaaagtgcgggtatcgaacaggattagataccctggtagtccgcacggtaaacgatggatgcccgttgtcaggctgtttcagcctggtgaccaagcgaaagcattaagcatcccacctggggagtacgccggcaacggtgaaactcaaaggaattgacgggggcccgcacaagcggaggaacatgtggtttaattcgatgatacgcgaggaaccttacccgggcttgaattgcagacgaacgattcagagatgatgaggcccttcggggcgtctgtgaaggtgctgcatggttgtcgtcagctcgtgccgtgaggtgtcggcttaagtgccataacgagcgcaacccctttcttcagttgccatcaggtgatgctgggcactctgtagatactgccaccgcaaggtgtgaggaaggtggggatgacgtcaaatcagcacggcccttacgtccggggctacacacgtgttacaatggccggtacagagagttgattttgtgcaaacacgatctaatccttaaatccggtcccagttcggactggggtctgcaacccgaccccacgaagctggattcgctagtaatcgcgcatcagccatggcgcggtgaatacgttcccgggccttgtacacaccgcccgtcaagccatgaaagccgggggtgcctgaagtctgtgaccgcaaggaacggcctagggcaaaaccggtgattggggctaagtcgtaacaaggtagccgtaccggaaggtgcggctggaacacctcctttct

SEQ ID NO: 55 - DNA, 1532 bp, Prevotella oris
  misc_feature: Prevotella oris F0302
tacaatggagagtttgatcctggctcaggatgaacgctggctacaggcttaacacatgcaagtcgaggggaaacgacggggaagcttgcttccttgggcgtcgaccggcgcacgggtgagtaacgcgtatccaacctgcctctgactgagggataacccgtcgaaagtcggcctaatacctcatggcatcgtctgcgggcatccaacgacgattaaagatttcatcggtcagggatggggatgcgtctgattagcttgttggcggggtaacggcccaccaaggctacgatcagtaggggttctgagaggaaggtcccccacattggaactgagacacggtccaaactcctacgggaggcagcagtgaggaatattggtcaatgggcgagagcctgaaccagccaagtagcgtgcaggaagacggccctatgggttgtaaactgcttttatgcggggataaagtgagggacgtgtccttcattgcaggtaccgcatgaataaggaccggctaattccgtgccagcagccgcggtaatacggaaggtccgggcgttatccggatttattgggtttaaagggagcgtaggccgtggattaagcgtgttgtgaaatgcaggtgctcaacgtctgcactgcagcgcgaactggtccacttgagtgtgcgcaacgcaggcggaattcgtcgtgtagcggtgaaatgcttagatatgacgaagaactccgattgcgaaggcagcttgcgggagcacaactgacgctgaagctcgaaagtgcgggtatcgaacaggattagataccctggtagtccgcacggtaaacgatggatgcccgttgtcaggctgtttcagcctggtgaccaagcgaaagcattaagcatcccacctggggagtacgccggcaacggtgaaactcaaaggaattgacgggggcccgcacaagcggaggaacatgtggtttaattcgatgatacgcgaggaaccttacccgggcttgaattgcagacgaacgattcagagatgatgaggcccttcggggcgtctgtgaaggtgctgcatggttgtcgtcagctcgtgccgtgaggtgtcggcttaagtgccataacgagcgcaacccctttcttcagttgccatcaggtgatgctgggcactctgtagatactgccaccgcaaggtgtgaggaaggtggggatgacgtcaaatcagcacggcccttacgtccggggctacacacgtgttacaatggccggtacagagagttgattttgtgcaaacacgatctaatccttaaatccggtcccagttcggactggggtctgcaacccgaccccacgaagctggattcgctagtaatcgcgcatcagccatggcgcggtgaatacgttcccgggccttgtacacaccgcccgtcaagccatgaaagccgggggtgcctgaagtctgtgaccgcaaggaacggcctagggcaaaaccggtgattggggctaagtcgtaacaaggtagccgtaccggaaggtgcggctggaacacctcctt

SEQ ID NO: 56 - DNA, 1533 bp, Prevotella oris
  misc_feature: Prevotella oris F0302
tacaatggagagtttgatcctggctcaggatgaacgctggctacaggcttaacacatgcaagtcgaggggaaacgacggggaagcttgcttccttcgggcgtcgaccggcgcacgggtgagtaacgcgtatccaacctgcctctgactgagggataacccgtcgaaagtcggcctaatacctcatggcatcgtctgcgggcatccaacgacgattaaagatttcatcggtcagggatggggatgcgtctgattagcttgttggcggggtaacggcccaccaaggctacgatcagtaggggttctgagaggaaggtcccccacattggaactgagacacggtccaaactcctacgggaggcagcagtgaggaatattggtcaatgggcgagagcctgaaccagccaagtagcgtgcaggaagacggccctatgggttgtaaactgcttttatgcggggataaagtgagggacgtgtccttcattgcaggtaccgcatgaataaggaccggctaattccgtgccagcagccgcggtaatacggaaggtccgggcgttatccggatttattgggtttaaagggagcgtaggccgtggattaagcgtgttgtgaaatgcaggtgctcaacgtctgcactgcagcgcgaactggtccacttgagtgtgcgcaacgcaggcggaattcgtcgtgtagcggtgaaatgcttagatatgacgaagaactccgattgcgaaggcagcttgcgggagcacaactgacgctgaagctcgaaagtgcgggtatcgaacaggattagataccctggtagtccgcacggtaaacgatggatgcccgttgtcaggctgtttcagcctggtgaccaagcgaaagcattaagcatcccacctggggagtacgccggcaacggtgaaactcaaaggaattgacgggggcccgcacaagcggaggaacatgtggtttaattcgatgatacgcgaggaaccttacccgggcttgaattgcagacgaacgattcagagatgatgaggcccttcggggcgtctgtgaaggtgctgcatggttgtcgtcagctcgtgccgtgaggtgtcggcttaagtgccataacgagcgcaacccctttcttcagttgccatcaggtgatgctgggcactctgtagatactgccaccgcaaggtgtgaggaaggtggggatgacgtcaaatcagcacggcccttacgtccggggctacacacgtgttacaatggccggtacagagagttgattttgtgcaaacacgatctaatccttaaatccggtcccagttcggactggggtctgcaacccgaccccacgaagctggattcgctagtaatcgcgcatcagccatggcgcggtgaatacgttcccgggccttgtacacaccgcccgtcaagccatgaaagccgggggtgcctgaagtctgtgaccgcaaggaacggcctagggcaaaaccggtgattggggctaagtcgtaacaaggtagccgtaccggaaggtgcggctggaacacctcctt

SEQ ID NO: 57 - DNA, 1533 bp, Prevotella oris; identical to SEQ ID NO: 56
  misc_feature: Prevotella oris F0302

SEQ ID NO: 58 - DNA, 1550 bp, Staphylococcus aureus
  misc_feature: Staphylococcus aureus ACM 2434
tggagagtttgatcctggctcaggatgaacgctggcggcgtgcctaatacatgcaagtcgagcgaacggacgagaagcttgcttctctgatgttagcggcggacgggtgagtaacacgtggataacctacctataagactgggataacttcgggaaaccggagctaataccggataatattttgaaccgcatggttcaaaagtgaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacatcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttaggagccagccgtcgaaggtgggacaaatgattggggtgaagtcgtaacaaggtagccgtatcggaaggtgcggctggatcacctcctttct

SEQ ID NO: 59 - DNA, 1497 bp, Staphylococcus aureus
  misc_feature: Staphylococcus aureus ACM 2434
atcctggctcaggatgaacgctggcggcgtgcctaatacatgcaagtcgagcgaacggacgagaagcttgcttctctgatgttagcggcggacgggtgagtaacacgtggataacctacctataagactgggataacttcgggaaaccggagctaataccggataatattttgaaccgcatggttcaaaagtgaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttaggagctagccgtcgaaggtgggacaaatgattggggtgaagtcgtaaca

SEQ ID NO: 60 - DNA, 1550 bp, Staphylococcus aureus
  misc_feature: Staphylococcus aureus ACM 2434
tggagagtttgatcctggctcaggatgaacgctggcggcgtgcctaatacatgcaagtcgagcgaacggacgagaagcttgcttctctgatgttagcggcggacgggtgagtaacacgtggataacctacctataagactgggataacttcgggaaaccggagctaataccggataatattttgaaccgcatggttcaaaagtgaaagacggtcttgctgtcacttatagatggatccgcgctgcattagctagttggtaaggtaacggcttaccaaggcaacgatacgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtattgtacacaccgcccgtcacaccacgagagtttgtaacacccgaagccggtggagtaaccttttaggagctagccgtcgaaggtgggacaaatgattggggtgaagtcgtaacaaggtagccgtatcggaaggtgcggctggatcacctcctttct

SEQ ID NO: 61 - DNA, 1435 bp, Staphylococcus aureus
  misc_feature: Staphylococcus aureus ACM 2434
tgaacgctggcggcgtgcctaatacatgcaagtcgagcgaacggacgagaagcttgcttctctgatgttagcggcggacgggtgagtaacacgtggataacctacctataagactgggataacttcgggaaaccggagctaataccggataatattttgaaccgcatggttcaaaagtgaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcgtggagcatgtggtttaattcgaagcaacgcgaagaaccttaccaaatcttgacatcctttgacaactctagagatagagccttcttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgaagagtttgtaacacccgaagccggtggagtaaccttttaggag

SEQ ID NO: 62 - DNA, 1476 bp, Staphylococcus aureus
  misc_feature: Staphylococcus aureus ACM 2434
aggatgaacgctggcggcgtgcctaatacatgcaagtcgagcgaacggacgagaagcttgcttctctgatgttagcggcggacgggtgagtaacacgtggataacctacctataagactgggataacttcgggaaaccggagctaataccggataatattttgaaccgcatggttcaaaagtgaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttaggagctagccgtcgaaggtgggacaaatgattggggtg

SEQ ID NO: 63 - DNA, 1556 bp, Staphylococcus aureus
  misc_feature: Staphylococcus aureus ACM 2434
tttttatggagagtttgatcctggctcaggatgaacgctggcggcgtgcctaatacatgcaagtcgagcgaacggacgagaagcttgcttctctgatgttagcggcggacgggtgagtaacacgtggataacctacctataagactgggataacttcgggaaaccggagctaataccggataatattttgaaccgcatggttcaaaagtgaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttaggagctagccgtcgaaggtgggacaaatgattggggtgaagtcgtaacaaggtagccgtatcggaaggtgcggctggatcacctcctttct

SEQ ID NO: 64 - DNA, 1556 bp, Staphylococcus aureus
  misc_feature: Staphylococcus aureus ACM 2434
tttttatggagagtttgatcctggctcaggatgaacgctggcggcgtgcctaatacatgcaagtcgagcgaacggacgagaagcttgcttctctgatgttagcggcggacgggtgagtaacacgtggataacctacctataagactgggataacttcgggaaaccggagctaataccggataatattttgaaccgcatggttcaaaagtgaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttacggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttaggagctagccgtcgaaggtgggacaaatgattggggtgaagtcgtaacaaggtagccgtatcggaaggtgcggctggatcacctcctttct

SEQ ID NO: 65 - DNA, 1556 bp, Staphylococcus aureus; identical to SEQ ID NO: 64
  misc_feature: Staphylococcus aureus ACM 2434

SEQ ID NO: 66 - DNA, 1477 bp, Staphylococcus aureus
  misc_feature: Staphylococcus aureus ACM 2434
gatgaacgctggcggcgtgcctaatacatgcaagtcgagcgaacggacgagaagcttgcttctctgatgttagcggcggacgggtgagtaacacgtggataacctacctataagactgggataacttcgggaaaccggagctaataccggataatattttgaaccgcatggttcaaaagtgaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttaggagctagccgtcgaaggtgggacaaatgattggggtgaag

SEQ ID NO: 67 - DNA, 1545 bp, Staphylococcus aureus
  misc_feature: Staphylococcus aureus ACM 2434
agagtttgatcctggctcaggatgaacgctggcggcgtgcctaatacatgcaagtcgagcgaacggacgagaagcttgcttctctgatgttagcggcggacgggtgagtaacacgtggataacctacctataagactgggataacttcgggaaaccggagctaataccggataatattttgaaccgcatggttcaaaagtgaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttacggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttaggagctagccgtcgaaggtgggacaaatgattggggtgaagtcgtaacaaggtagccgtatcggaaggtgcggctggatcacctccttt

SEQ ID NO: 68 - DNA, 1545 bp, Staphylococcus aureus; identical to SEQ ID NO: 67
  misc_feature: Staphylococcus aureus ACM 2434

SEQ ID NO: 69 - DNA, 1545 bp, Staphylococcus aureus
  misc_feature: Staphylococcus aureus ACM 2434
agagtttgatcctggctcaggatgaacgctggcggcgtgcctaatacatgcaagtcgagcgaacggacgagaagcttgcttctctgatgttagcggcggacgggtgagtaacacgtggataacctacctataagactgggataacttcgggaaaccggagctaataccggataatattttgaaccgcatggttcaaaagtgaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttaggagctagccgtcgaaggtgggacaaatgattggggtgaagtcgtaacaaggtagccgtatcggaaggtgcggctggatcacctccttt

SEQ ID NO: 70 - DNA, 1557 bp, Staphylococcus aureus
  misc_feature: Staphylococcus aureus ACM 2434
ttttatggagagtttgatcctggctcaggatgaacgctggcggcgtgcctaatacatgcaagtcgagcgaacggacgagaagcttgcttctctgatgttagcggcggacgggtgagtaacacgtggataacctacctataagactgggataacttcgggaaaccgtagctaataccggataatattttgaaccgcatggttcaaaagtgaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttaggagctagccgtcgaaggtgggacaaatgattggggtgaagtcgtaacaaggtagccgtatcggaaggtgcggctggatcacctcctttctaa

SEQ ID NO: 71 - DNA, 1557 bp, Staphylococcus aureus
  misc_feature: Staphylococcus aureus ACM 2434
ttttatggagagtttgatcctggctcaggatgaacgctggcggcgtgcctaatacatgcaagtcgagcgaacggacgagaagcttgcttctctgatgttagcggcggacgggtgagtaacacgtggataacctacctataagactgggataacttcgggaaaccggagctaataccggataatattttgaaccgcatggttcaaaagtgaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttaggagctagccgtcgaaggtgggacaaatgattggggtgaagtcgtaacaaggtagccgtatcggaaggtgcggctggatcacctcctttctaa

SEQ ID NO: 72 - DNA, 1557 bp, Staphylococcus aureus; identical to SEQ ID NO: 71
  misc_feature: Staphylococcus aureus ACM 2434

SEQ ID NO: 73 - DNA, 1557 bp, Staphylococcus aureus
  misc_feature: Staphylococcus aureus ACM 2434
ttttatggagagtttgatcctggctcaggatgaacgctggcggcgtgcctaatacatgcaagtcgagcgaacggacgagaagcttgcttctctgatgttagcggcggacgggtgagtaacacgtggataacctacctataagactgggataacttcgggaaaccggagctaataccggataatattttgaaccgcatggttcaaaagtgaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagctttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgtgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttaggagctagccgtcgaaggtgggacaaatgattggggtgaagtcgtaacaaggtagccgtatcggaaggtgcggctggatcacctcctttctaa

SEQ ID NO: 74 - DNA, 1557 bp, Staphylococcus aureus
  misc_feature: Staphylococcus aureus ACM 2434
ttttatggagagtttgatcctggctcaggatgaacgctggcggcgtgcctaatacatgcaagtcgagcgaacggacgagaagcttgcttctctgatgttagcggcggacgggtgagtaacacgtggataacctacctataagactgggataacttcgggaaaccggagctaataccggatgatattttgaaccgcatggttcaaaagtgaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttaggagctagccgtcgaaggtgggacaaatgattggggtgaagtcgtaacaaggtagccgtatcggaaggtgcggctggatcacctcctttctaa

SEQ ID NO: 75 - DNA, 1557 bp, Staphylococcus aureus; identical to SEQ ID NO: 71
  misc_feature: Staphylococcus aureus ACM 2434

SEQ ID NO: 76 - DNA, 1276 bp, Staphylococcus aureus
  misc_feature: Staphylococcus aureus ACM 2434
ttttatggagagtttgatcctggctcaggatgaacgctggcggcgtgcctaatacatgcaagtcgagcgaacggacgagaagcttgcttctctgatgttagcggcggacgggtgagtaacacgtggataacctacctataagactgggataacttcgggaaaccggagctaataccggataatattttgaaccgcatggttcaaaagtgaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccg

SEQ ID NO: 77 - DNA, 1517 bp, Staphylococcus aureus
  misc_feature: Staphylococcus aureus C-243
gagagtttgatcctggctcaggatgaacgctggcggcgtgcctaatacatgcaagtcgagcgaacggacgagaagcttgcttctctgatgttagcggcggacgggtgagtaacacgtggataacctacctataagactgggataacttcgggaaaccggagctaataccggatgatattttgaaccgcatggttcaaaagtgaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttaggagctagccgtcgaaggtgggacaaatgattggggtgaagtcgtaacaaggtagccgta

SEQ ID NO: 78 - DNA, 1515 bp, Staphylococcus aureus
  misc_feature: Staphylococcus aureus C-243
gagtttgatcctggctcaggatgaacgctggcggcgtgcctaatacatgcaagtcgagcgaacggacgagaagcttgcttctctgatgttagcggcggacgggtgagtaacacgtggataacctacctataagactgggataacttcgggaaaccggagctaataccggataatattttgaaccgcatggttcaaaagtgaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttaggagctagccgtcgaaggtgggacaaatgattggggtgaagtcgtaacaaggtagccgta

SEQ ID NO: 79 - DNA, 1556 bp, Staphylococcus aureus; identical to SEQ ID NO: 63
  misc_feature: Staphylococcus aureus MRSA252

SEQ ID NO: 80 - DNA, 1557 bp, Staphylococcus aureus; identical to SEQ ID NO: 71
  misc_feature: Staphylococcus aureus MRSA252

SEQ ID NO: 81 - DNA, 1557 bp, Staphylococcus aureus; identical to SEQ ID NO: 71
  misc_feature: Staphylococcus aureus MRSA252

SEQ ID NO: 82 - DNA, 1558 bp, Staphylococcus aureus
  misc_feature: Staphylococcus aureus MRSA252
ttttatggagagtttgatcctggctcaggatgaacgctggcggcgtgcctaatacatgcaagtcgagcgaacggacgagaagcttgcttctctgatgttagcggcggacgggtgagtaacacgtggataacctacctataagactgggataacttcgggaaaccggagctaataccggataatattttgaaccgcatggttcaaaagtgaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcgggggggcaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttaggagctagccgtcgaaggtgggacaaatgattggggtgaagtcgtaacaaggtagccgtatcggaaggtgcggctggatcacctcctttctaa

SEQ ID NO: 83 - DNA, 1557 bp, Staphylococcus aureus; identical to SEQ ID NO: 71
  misc_feature: Staphylococcus aureus MRSA252

SEQ ID NO: 84 - DNA, 1556 bp, Staphylococcus aureus
  misc_feature: Staphylococcus aureus MRSA252
ttgtatggagagtttgatcctggcccatgatgaacgctggcggcgtgtcctatacatgccagtcgagcgaacggacgaaagcttgcttctctgatgttagcggcggacgggtgagtaacacgtggataacctacctataagactgggataacttcgggaaaccggagctaataccggataatattttgaaccgcatggttcaaaagtgaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttaggagctagccgtcgaaggtgggacaaatgattggggtgaagtcgtaacaaggtagccgtatcggaaggtgcggctggatcacctcctttctaa

SEQ ID NO: 85 - DNA, 1556 bp, Staphylococcus aureus
  misc_feature: Staphylococcus aureus MSSA476
tttttatggagagtttgatcctggctcaggatgaacgctggcggcgtgcctaatacatgcaagtcgagcgaacggacgagaagcttgcttctctgatgttagcggcggacgggtgagtaacacgtggataacctacctataagactgggataacttcgggaaaccggagctaataccggataatattttgaaccgcatggttcgaaagtgaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttaggagctagccgtcgaaggtgggacaaatgattggggtgaagtcgtaacaaggtagccgtatcggaaggtgcggctggatcacctcctttct

SEQ ID NO: 86 - DNA, 1557 bp, Staphylococcus aureus; identical to SEQ ID NO: 71
  misc_feature: Staphylococcus aureus MSSA476

SEQ ID NO: 87 - DNA, 1557 bp, Staphylococcus aureus
  misc_feature: Staphylococcus aureus MSSA476
ttttatggagagtttgatcctggctcaggatgaacgctggcggcgtgcctaatacatgcaagtcgagcgaacggacgagaagcttgcttctctgatgttagcggcggacgggtgagtaacacgtggataacctacctataagactgggataacttcgggaaaccggagctaataccggataatattttgaaccgcatggttcaaaagtgaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttaggaactagccgtcgaaggtgggacaaatgattggggtgaagtcgtaacaaggtagccgtatcggaaggtgcggctggatcacctcctttctaa

SEQ ID NO: 88 - DNA, 1557 bp, Staphylococcus aureus
  misc_feature: Staphylococcus aureus MSSA476
ttttatggagagtttgatcctggctcaggatgaacgctggcggcgtgcctaatacatgcaagtcgagcgaacggacgagaagcttgcttctctgatgttagcggcggacgggtgagtaacacgtggataacctacctataagactgggataacttcgggaaaccggagctaataccggataatattttgaaccgcatggttcaaaagtgaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagctttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttaggagctagccgtcgaaggtgggacaaatgattggggtgaagtcgtaacaaggtagccgtatcggaaggtgcggctggatcacctcctttctaa

SEQ ID NO: 89 - DNA, 1557 bp, Staphylococcus aureus
  misc_feature: Staphylococcus aureus MSSA476
ttttatggagagtttgatcctggctcaggatgaacgctggcggcgtgcctaatacatgcaagtcgagcgaacggatgagaagcttgcttctctgatgttagcggcggacgggtgagtaacacgtggataacctacctataagactgggataacttcgggaaaccggagctaataccggataatattttgaaccgcatggttcaaaagtgaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagctttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtagggatgacgtcaaatcatcatgccccttatgatttgggctacacacgtgctacaatggacaatacaaagggcagcgaaaccgtgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttaggagctagccgtcgaaggtgggacaaatgattggggtgaagtcgtaacaaggtagccgtatcggaaggtgcggctggatcacctcctttctaa

SEQ ID NO: 90 - DNA, 1557 bp, Staphylococcus aureus
  misc_feature: Staphylococcus aureus MSSA476
ttttatggagagtttgatcctggctcaggatgaacgctggcggcgtgcctaatacatgcaagtcgagcgaacggacgagaagcttgcttctctgatgttagcggcggacgggtgagtaacacgtggataacctacctataagactgggataacttcgggaaaccggagctaataccggataatattttgaaccgcatggttcgaaagtgaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttaggagctagccgtcgaaggtgggacaaatgattggggtgaagtcgtaacaaggtagccgtatcggaaggtgcggctggatcacctcctttctaa

SEQ ID NO: 91 - DNA, 1557 bp, Staphylococcus aureus
  misc_feature: Staphylococcus aureus MSSA476
ttttatggagagtttgatcctggctcaggatgaacgctggcggcgtgcctaatacatgcaagtcgagcgaacggacgagaagcttgcttctctgatgttagcggcggacgggtgagtaacacgtggataacctacctataagactgggataacttcgggaaaccggagctaataccggataatattttgaaccgcatggttcaaaagtgaaagacggtcttactgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagctttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttaggagctagccgtcgaaggtgggacaaatgattggggtgaagtcgtaacaaggtagccgtatcggaaggtgcggctggatcacctcctttctaa

SEQ ID NO: 92 - DNA, 1557 bp, Staphylococcus aureus; identical to SEQ ID NO: 88
  misc_feature: Staphylococcus aureus MSSA476

SEQ ID NO: 93 - DNA, 1557 bp, Staphylococcus aureus; identical to SEQ ID NO: 71
  misc_feature: Staphylococcus aureus MSSA476

SEQ ID NO: 94 - DNA, 1557 bp, Staphylococcus aureus; identical to SEQ ID NO: 71
  misc_feature: Staphylococcus aureus MSSA476

SEQ ID NO: 95 - DNA, 1557 bp, Staphylococcus aureus; identical to SEQ ID NO: 88
  misc_feature: Staphylococcus aureus MSSA476

SEQ ID NO: 96 - DNA, 1557 bp, Staphylococcus aureus; identical to SEQ ID NO: 71
  misc_feature: Staphylococcus aureus MSSA476

SEQ ID NO: 97 - DNA, 1557 bp, Staphylococcus aureus; identical to SEQ ID NO: 88
  misc_feature: Staphylococcus aureus MSSA476

SEQ ID NO: 98 - DNA, 1550 bp, Staphylococcus aureus
  misc_feature: Staphylococcus aureus MU3
tggagagtttgatcctggctcaggatgaacgctggcggcgtgcctaatacatgcaagtcgagcgaacggatgagaagcttgcttctctgatgttagcggcggacgggtgagtaacacgtggataacctacctataagactgggataacttcgggaaaccggagctaataccggataatattttgaaccgcatggttcaaaagtgaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttaggagctagccgtcgaaggtgggacaaatgattggggtgaagtcgtaacaaggtagccgtatcggaaggtgcggctggatcacctcctttct

SEQ ID NO: 99 - DNA, 1556 bp, Staphylococcus aureus
  misc_feature: Staphylococcus aureus MU3
tttttatggagagtttgatcctggctcaggatgaacgctggcggcgtgcctaatacatgcaagtcgagcgaacggatgagaagcttgcttctctgatgttagcggcggacgggtgagtaacacgtggataacctacctataagactgggataacttcgggaaaccggagctaataccggataatattttgaaccgcatggttcaaaagtgaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttaggagctagccgtcgaaggtgggacaaatgattggggtgaagtcgtaacaaggtagccgtatcggaaggtgcggctggatcacctcctttct

SEQ ID NO: 100 - DNA, 1557 bp, Staphylococcus aureus
  misc_feature: Staphylococcus aureus MU3
ttttatggagagtttgatcctggctcaggatgaacgctggcggcgtgcctaatacatgcaagtcgagcgaacggacgagaagcttgcttctctgatgttagcggcggacgggtgagtaacacgtggataacctacctataagactgggataacttcgggaaaccggagctaataccggataatattttgaaccgcatggttcaaaagtgaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttcctcttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttaggagctagccgtcgaaggtgggacaaatgattggggtgaagtcgtaacaaggtagccgtatcggaaggtgcggctggatcacctcctttctaa

SEQ ID NO: 101 - DNA, 1557 bp, Staphylococcus aureus
  misc_feature: Staphylococcus aureus MU3
ttttatggagagtttgatcctggctcaggatgaacgctggcggcctgcctaatacatgcaagtcgagcgaacggacgagaagcttgcttctctgatgttagcggcggacgggtgagtaacacgtggataacctacctataagactgggataacttcgggaaaccggagctaataccggataatattttgaaccgcatggttcaaaagtgaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagctttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgtgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttaggagctagccgtcgaaggtgggacaaatgattggggtgaagtcgtaacaaggtagccgtatcggaaggtgcggctggatcacctcctttctaa

SEQ ID NO: 102 - DNA, 1557 bp, Staphylococcus aureus
  misc_feature: Staphylococcus aureus MU3
ttttatggagagtttgatcctggctcaggatgaacgctggcggcgtgcctaatacatgcaagtcgagcgaacggacgagaagcttgcttctctgatgttagcggcggacgggtgagtaacacgtggataacctacctataagactgggataacttcgggaaaccggagctaataccggataatattttgaaccgcatggttcaaaagtaaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttaggagctagccgtcgaaggtgggacaaatgattggggtgaagtcgtaacaaggtagccgtatcggaaggtgcggctggatcacctcctttctaa

SEQ ID NO: 103 - DNA, 1557 bp, Staphylococcus aureus; identical to SEQ ID NO: 71
  misc_feature: Staphylococcus aureus MU3

SEQ ID NO: 104 - DNA, 1135 bp, Staphylococcus aureus
  misc_feature: Staphylococcus aureus MU3
ttttatggagagtttgatcctggctcaggatgaacgctggcggcgtgcctaatacatgcaagtcgagcgaacggacgagaagcttgcttctctgatgttagcggcggacgggtgagtaacacgtggataacctacctataagactgggataacttcgggaaaccggagctaataccggataatattttgaaccgcatggttcaaaagtgaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcgggggacaaagtgacaggtggtgcatggttgtcgtcagctcgtgtcgtgagatgttgggttaagtcccgcaacgagcgcaacccttaagcttag

SEQ ID NO: 105 - DNA, 1492 bp, Staphylococcus caprae
  misc_feature: Staphylococcus caprae 143.22
agagtttgatcctggctcaggatgaacgctggcggcgtgcctaatacatgcaagtcgagcgaacagacgaggagcttgctcctctgacgttagcggcggacgggtgagtaacacgtggataacctacctataagactgggataacttcgggaaaccggagctaataccggataacatgttgaaccgcatggttcaacagtgaaagacggtcttgctgtcacttatagatggatccgcgccgcattagctagttggtaaggtaacggcttaccaaggcaacgatgcgtagccgacctgagagggtgatcggccacactggaactgagacacggtccagactcctacgggaggcagcagtagggaatcttccgcaatgggcgaaagcctgacggagcaacgccgcgtgagtgaagaaggtcttcggatcgtaaaactctgttattagggaagaacaaatgtgtaagtaactatgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgacccctctagagatagagttttccccttcgggggacagagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttggagctagccgtcgaaggtgggacaaatgattggggt

SEQ ID NO: 106 - DNA, 1470 bp, Staphylococcus caprae
  misc_feature: Staphylococcus caprae 143.22
aacgctggcggcgtgcctaatacatgcaagtcgagcgaacagacgaggagcttgctcctctgacgttagcggcggacgggtgagtaacacgtggataacctacctataagactgggataacttcgggaaaccggagctaataccggataacatgttgaaccgcatggttcaacagtgaaagacggtcttgctgtcacttatagatggatccgcgccgcattagctagttggtaaggtaacggcttaccaaggcaacgatgcgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaatgtgtaagtaactatgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgacccctctagagatagagttttccccttcgggggacagagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggtggcgaaaccgcgaggtcaagcaaatcccataaagttgttctcagttcggattgtagtctgcaactcgactacatgaagctggaatcgctagtaatcgtggatcagcatgccacggtgaatacgttcccgggtcttgtacacaccgcccgtcacaccacgagagtttgtaacacccgaagccggtggagtaaccttttaggagctagccgtcgaaggtgggacaaatgattggggtg

SEQ ID NO: 107 - DNA, 1556 bp, Staphylococcus caprae
  misc_feature: Staphylococcus caprae M23864 W1
ttttatggagagtttgatcctggctcaggatgaacgctggcggcgtgcctaatacatgcaagtcgagcgaacagacgaggagcttgctcctctgacgttagcggcggacgggtgagtaacacgtggataacctacctataagactgggataacttcgggaaaccggagctaataccggataacatgttgaaccgcatggttcaacagtgaaagacggtcttgctgtcacttatagatggatccgcgccgcattagctagttggtaaggtaacggcttaccaaggcaacgatgcgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaatgtgtaagtaactatgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgacccctctagagatagagttttccccttcgggggacagagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggtggcgaaaccgcgaggtcaagcaaatcccataaagttgttctcagttcggattgtagtctgcaactcgactacatgaagctggaatcgctagtaatcgtggatcagcatgccacggtgaatacgttcccgggtcttgtacacaccgcccgtcacaccacgagagtttgtaacacccgaagccggtggagtaaccttttggagctagccgtcgaaggtgggacaaatgattggggtgaagtcgtaacaaggtagccgtatcggaaggtgcggctggatcacctcctttctaa

SEQ ID NO: 108 - DNA, 1549 bp, Staphylococcus caprae
  misc_feature: Staphylococcus caprae M23864 W1
tggagagtttgatcctggctcaggatgaacgctggcggcgtgcctaatacatgcaagtcgagcgaacagacgaggagcttgctcctctgacgttagcggcggacgggtgagtaacacgtggataacctacctataagactgggataacttcgggaaaccggagctaataccggataacatgttgaaccgcatggttcaacagtgaaagacggtcttgctgtcacttatagatggatccgcgccgcattagctagttggtaaggtaacggcttaccaaggcaacgatgcgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaatgtgtaagtaactatgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgacccctctagagatagagttttccccttcgggggacagagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggtggcgaaaccgcgaggtcaagcaaatcccataaagttgttctcagttcggattgtagtctgcaactcgactacatgaagctggaatcgctagtaatcgtggatcagcatgccacggtgaatacgttcccgggtcttgtacacaccgcccgtcacaccacgagagtttgtaacacccgaagccggtggagtaaccttttggagctagccgtcgaaggtgggacaaatgattggggtgaagtcgtaacaaggtagccgtatcggaaggtgcggctggatcacctcctttct

SEQ ID NO: 109 - DNA, 1549 bp, Staphylococcus caprae; identical to SEQ ID NO: 108
  misc_feature: Staphylococcus caprae M23864 W1

SEQ ID NO: 110 - DNA, 1555 bp, Staphylococcus caprae
  misc_feature: Staphylococcus caprae M23864 W1
tttttatggagagtttgatcctggctcaggatgaacgctggcggcgtgcctaatacatgcaagtcgagcgaacagacgaggagcttgctcctctgacgttagcggcggacgggtgagtaacacgtggataacctacctataagactgggataacttcgggaaaccggagctaataccggataacatgttgaaccgcatggttcaacagtgaaagacggtcttgctgtcacttatagatggatccgcgccgcattagctagttggtaaggtaacggcttaccaaggcaacgatgcgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaatgtgtaagtaactatgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgacccctctagagatagagttttccccttcgggggacagagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggtggcgaaaccgcgaggtcaagcaaatcccataaagttgttctcagttcggattgtagtctgcaactcgactacatgaagctggaatcgctagtaatcgtggatcagcatgccacggtgaatacgttcccgggtcttgtacacaccgcccgtcacaccacgagagtttgtaacacccgaagccggtggagtaaccttttggagctagccgtcgaaggtgggacaaatgattggggtgaagtcgtaacaaggtagccgtatcggaaggtgcggctggatcacctcctttct

SEQ ID NO: 111 - DNA, 1477 bp, Staphylococcus cohnii
  misc_feature: Staphylococcus cohnii CCS 85
aggatgaacgctggcggcgtgcctaatacatgcaagtcgagcgaacagataaggagcttgctcctttgacgttagcggcggacgggtgagtaacacgtgggtaacctacctataagactggaataactccgggaaaccggggctaatgccggataacatttagaaccgcatggttctaaagtgaaagatggttttgctatcacttatagatggacccgcgccgtattagctagttggtaaggtaacggcttaccaaggcaacgatacgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaatgtgtaagtaactatgcacgtcttgacggtacctaatcagaaagccacggctaactacgtgccagcagccgcggtaatacgtaggtggcaagcgttatccggaattattgggcgtaaagcgcgcgtaggcggtttcttaagtctgatgtgaaagcccacggctcaaccgtggagggtcattggaaactgggaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacaactctagagatagagccttccccttcgggggacaaagtgacaggtggtgcatggttgtcgtcagctcgtgtcgtgagatgttgggttaagtcccgcaacgagcgcaacccttaaacttagttgccagcatttagttgggcactctaagttgactgccggtgacaaaccggaggaaggtggggatgacgtcaaatcatcatgccccttatgatttgggctacacacgtgctacaatggacaatacaaagggcagctaaaccgcgaggtcatgcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccatttatggagctagccgtcgaaggtgggacaaatgattggggtg

SEQ ID NO: 112 - DNA, 1537 bp, Staphylococcus devriesei
  misc_feature: Staphylococcus devriesei KS SP60
tagagtttgatcctggctcaggatgaacgctggcggcgtgcctaatacatgcaagtcgagcgaacagataaggagcttgctcctttgacgttagcggcggacgggtgagtaacacgtgggtaacctacctataagactgggataacttcgggaaaccggagctaataccggataatatttcgaaccgcatggttcgatagtgaaagatggttttgctatcacttatagatggatccgcgccgtattagctagttggtaaggtaacggcttaccaaggcaacgatacgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatacgtgtaagtaactatgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaatcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgaccgctctagagatagagttttccccttcgggggacaaagtgacaggtggtgcatggttgtcgtcagctcgtgtcgtgagatgttgggttaagtcccgcaacgagcgcaacccttaaacttagttgccagcattcagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccattttggagctagccgtcgaaggtgggacaaatgattggggtgaagtcgtaacaaggtagccgtatcggaaggtgcggctggatc

SEQ ID NO: 113 - DNA, 1549 bp, Staphylococcus epidermidis
  misc_feature: Staphylococcus epidermidis WHO 12
tggagagtttgatcctggctcaggatgaacgctggcggcgtgcctaatacatgcaagtcgagcgaacagacgaggagcttgctcctctgacgttagcggcggacgggtgagtaacacgtggataacctacctataagactgggataacttcgggaaaccggagctaataccggataatatattgaaccgcatggttcaatagtgaaagacggttttgctgtcacttatagatggatccgcgccgcattagctagttggtaaggtaacggcttaccaaggcaacgatgcgtagccgacctgagagggtgatcggccacactggaactgagacacggtccagactcctacgggaggcagcagtagggaatcttccgcaatgggcgaaagcttgacggagcaacgccgcgtgagtgatgaaggtcttcggatcgtaaaactctgttattagggaagaacaaatgtctaagtaactatgcacgtcttgacggtacctaatcagaaagccacggctaactacgtgccagcagccgcggtaatacgtaggtggcaagcgttgtccggaatcattgggcgtaaagcgcgcgtaggcggtttcttaagtctgatgtgaaagcccacggctcaaccgtggagggtcattggaaactggaggacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgatccctctagagatagaggtttccccttcgggggacagagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactatatgaagctggaatcgctagtaatcgtagatcagcatgctacggtgaatacgttcccgggtcttgtacacaccgcccgtcacaccacgagagtttgtaacacccgaagccggtggagtaaccatttggagctagccgtcgaaggtgggacaaatgattggggtgaagtcgtaacaaggtagccgtatcggaaggtgcggctggatcacctcctttct

SEQ ID NO: 114 - DNA, 1549 bp, Staphylococcus epidermidis; identical to SEQ ID NO: 113
  misc_feature: Staphylococcus epidermidis WHO 12

SEQ ID NO: 115 - DNA, 1476 bp, Staphylococcus epidermidis
  misc_feature: Staphylococcus epidermidis WHO 12
gatgaacgctggcggcgtgcctaatacatgcaagtcgagcgaacagacgaggagcttgctcctctgacgttagcggcggacgggtgagtaacacgtggataacctacctataagactgggataacttcgggaaaccggagctaataccggataatatattgaaccgcatggttcaatagtgaaagacggttttgctgtcacttatagatggatccgcgccgcattagctagttggtaaggtaacggcttaccaaggcaacgatgcgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaatgtgtaagtaactatgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgacccctctagagatagagttttccccttcgggggacagagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactatatgaagctggaatcgctagtaatcgtagatcagcatgctacggtgaatacgttcccgggtcttgtacacaccgcccgtcacaccacgagagtttgtaacacccgaagccggtggagtaaccatttggagctagccgtcgaaggtgggacaaatgattggggtgaag

SEQ ID NO: 116 - DNA, 1554 bp, Staphylococcus epidermidis
  misc_feature: Staphylococcus epidermidis WHO 12
ttttatggagagtttgatcctggctcaggatgaacgctggcggcgtgcctaatacatgcaagtcgagcgaacagatgaggagcttgctcctctgacgttagcggcggacgggtgagtaacacgtggataacctacctataagactgggataacttcgggaaaccggagctaataccggataatatattgaaccgcatggttcaatagtgaaagacggttttgctgtcacttatagatggatccgcgccgcattagctagttggtaaggtaacggcttaccaaggcaacgatgcgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaatgtgtaagtaactatgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgacccctctagagatagagttttccccttcgggggacagagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggtagcgaaaccgcgaggtcaagcaaatcccataaagttgttctcagttcggattgtagtctgcaactcgactatatgaagctggaatcgctagtaatcgtagatcagcatgctacggtgaatacgttcccgggtcttgtacacaccgcccgtcacaccacgagagtttgtaacacccgaagccggtggagtaaccatttggagctagccgtcgaaggtgggacaaatgattggggtgaagtcgtaacaaggtagccgtatcggaaggtgcggctggatcacctcctttct

SEQ ID NO: 117 - DNA, 1554 bp, Staphylococcus epidermidis
  misc_feature: Staphylococcus epidermidis WHO 12
ttttatggagagtttgatcctggctcaggatgaacgctggcggcgtgcctaatacatgcaagtcgagcgaacagacgaggagcttgctcctctgacgttagcggcggacgggtgagtaacacgtggataacctacctataagactgggataacttcgggaaaccggagctaataccggataatatattgaaccgcatggttcaatagtgaaagacggttttgctgtcacttatagatggatccgcgccgcattagctagttggtaaggtaacggcttaccaaggcaacgatgcgtagccgacctgagagggtgatcggccacactggaactgagacacggtccagactcctacgggaggcagcagtagggaatcttccgcaatgggcgaaagcttgacggagcaacgccgcgtgagtgatgaaggtcttcggatcgtaaaactctgttattagggaagaacaaatgtgtaagtaactatgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgacccctctagagatagagttttccccttcgggggacagagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactatatgaagctggaatcgctagtaatcgtagatcagcatgctacggtgaatacgttcccgggtcttgtacacaccgcccgtcacaccacgagagtttgtaacacccgaagccggtggagtaaccatttggagctagccgtcgaaggtgggacaaatgattggggtgaagtcgtaacaaggtagccgtatcggaaggtgcggctggatcacctcctttct

SEQ ID NO: 118 - DNA, 1422 bp, Staphylococcus epidermidis
  misc_feature: Staphylococcus epidermidis WHO 12
ttttatggagagtttgatcctggctcaggatgaacgctggcggcgtgcctaatacatgcaagtcgagcgaacagacgaggagcttgctcctctgacgttagcggcggacgggtgagtaacacgtggataacctacctataagactgggataacttcgggaaaccggagctaataccggataatatattgaaccgcatggttcaatagtgaaagacggttttgctgtcacttatagatggatccgcgccgcattagctagttggtaaggtaacggcttaccaaggcgacgatgcgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaatgtgtaagtaactatgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgatccctctagagatagaggtttccccttcgggggacagagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactatatgaagctggaatcgctagtaatcgtagatcagcatgctacggtgaatacgttcccgggtcttgtacacaccgcccgtcacacc

SEQ ID NO: 119 - DNA, 1554 bp, Staphylococcus epidermidis
  misc_feature: Staphylococcus epidermidis WHO 12
ttttatggagagtttgatcctggctcaggatgaacgctggcggcgtgcctaatacatgcaagtcgagcgaacagacgaggagcttgctcctctgacgttagcggcggacgggtgagtaacacgtggataacctacctataagactgggataacttcgggaaaccggagctaataccggataatatattgaaccgcatggttcaatagtgaaagacggttttgctgtcacttatagatggatccgcgccgcattagctagttggtaaggtaacggcttaccaaggcaacgatgcgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaatgtgtaagtaactatgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgacccctctagagatagagttttccccttcgggggacagagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactatatgaagctggaatcgctagtaatcgtagatcagcatgctacggtgaatacgttcccgggtcttgtacacaccgcccgtcacaccacgagagtttgtaacacccgaagccggtggagtaaccatttggagctagccgtcgaaggtggaacaaatgattggggtgaagtcgtaacaaggtagccgtatcggaaggtgcggctggatcacctcctttct

SEQ ID NO: 120 - DNA, 1468 bp, Staphylococcus hominis
  misc_feature: Staphylococcus hominis DM 122
gatgaacgctggcggcgtgcctaatacatgcaagtcgagcgaacagacgaggagcttgctcctttgacgttagcggcggacgggtgagtaacacgtgggtaacctacctataagactgggataacttcgggaaaccggagctaataccggataatatttcgaaccgcatggttcgatagtgaaagatggctctgctatcacttatagatggacctgcgccgtattagctagttggtaaggtaacggcttaccaaggcaacgatacgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaacgtgtaagtaactgtgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacccttctagagatagaagtttccccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccatttggagctagccgtcgaaggtgggacaaatgattg

SEQ ID NO: 121 - DNA, 1544 bp, Staphylococcus hominis
  misc_feature: Staphylococcus hominis DM 122
  misc_feature (11)..(12): n is a, c, g, or t
agagtttgatnntggctcaggatgaacgctggcggcgtgcctaatacatgcaagtcgagcgaacagacgaggagcttgctcctttgacgttagcggcggacgggtgagtaacacgtgggtaacctacctataagactgggataacttcgggaaaccggagctaataccggataatatttcgaaccgcatggttcgatagtgaaagatggctttgctatcacttatagatggacctgcgccgtattagctagttggtaaggtaacggcttaccaaggcaacgatacgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacaaacgtgtaagtaactgtgcacgtcttgacggtacctaatcagaaagccacggctaactacgtgccagcagccgcggtaatacgtaggtggcaagcgttatccggaattattgggcgtaaagcgcgcgtaggcggttttttaagtctgatgtgaaagcccacggctcaaccgtggagggtcattggaaactggaaaacttgagtgcagaagaggaaagtggaattccatgtgtagcggtgaaatgcgcagagatatggaggaacaccagtggcgaaggcgactttctggtctctaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgacccttctagagatagaagtttccttcgggggacaaagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggcagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccatttggagctagccgtcgaaggtgggacaaatgattggggtgaagtcgtaacaaggtagccgtatcggaaggtgcggctggatcacctcctttct

SEQ ID NO: 122 - DNA, 1552 bp, Streptococcus mitis
  misc_feature: Streptococcus mitis R 360
attttaatgagagtttgatcctggctcaggatgaacgctggcggcgtgcctaatacatgcaagtagaacgctgaagcttggtgcttgcaccgagcggatgagttgcgaacgggtgagtaacgcgtaggtaacctgcctcttagcgggggataactattggaaacgatagctaataccgcataaaagtcgatatcgcatgatgttgatttgaaaggtgcaaatgcatcactaagagatggacctgcgttgtattagctagttggtgaggtaacggctcaccaaggcgacgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgtaagagaagaacgagtgtgagagtggaaagttcacactgtgacggtaacttaccagaaagggacggctaactacgtgccagcagccgcggtaatacgtaggtcccgagcgttatccggatttattgggcgtaaagcgagcgcaggcggttagataagtctgaagttaaaggctgtggcttaaccatagtacgctttggaaactgtttaacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacaggattagataccctggtagtccacgccgtaaacgatgagtgctaggtgttgggtcctttccgggactcagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccctctgaccgctctagagatagagttttccttcgggacagaggtgacaggtggtgcatggttgtcgtcagctcgtgtcgtgagatgttgggttaagtcccgcaacgagcgcaacccctattgttagttgccatcattgagttgggcactctagcgagactgccggtaataaaccggaggaaggtggggatgacgtcaaatcatcatgccccttatgacctgggctacacacgtgctacaatggctggtacaacgagtcgcgagtcggtgacggcaagctaatctcttaaagccagtctcagttcggattgtaggctgcaactcgcctacatgaagtcggaatcgctagtaatcgcggatcagcacgtcgcggtgaatacgttcccgggccttgtacacaccgcccgtcacaccacgagagtttgtaacacccgaagtcggtgaggtaaccgtaaggagccagccgcctaaggtgggatagatgattggggtgaagtcgtaacaaggtagccgtatcggaaggtgcggctggatcacctcctttct

SEQ ID NO: 123 - DNA, 1555 bp, Streptococcus mitis
  misc_feature: Streptococcus mitis R 360
ttaatgagagtttgatcctggctcaggatgaacgctggcggcgtgcctaatacatgcaagtagaacgctgaagcttggtgcttgcaccgagcggatgagttgcgaacgggtgagtaacgcgtaggtaacctgcctcttagcgggggataactattggaaacgatagctaataccgcataaaagtcgatatcgcatgatgttgatttgaaaggtgcaaatgcatcactaagagatggacctgcgttgtattagctagttggtgaggtaacggctcaccaaggcgacgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgtaagagaagaacgagtgtgagagtggaaagttcacactgtgacggtaacttaccagaaagggacggctaactacgtgccagcagccgcggtaatacgtaggtcccgagcgttatccggatttattgggcgtaaagcgagcgcaggcggttagataagtctgaagttaaaggctgtggcttaaccatagtacgctttggaaactgtttaacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacaggattagataccctggtagtccacgccgtaaacgatgagtgctaggtgttgggtcctttccgggactcagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccctctgaccgctctagagatagagttttccttcgggacagaggtgacaggtggtgcatggttgtcgtcagctcgtgtcgtgagatgttgggttaagtcccgcaacgagcgcaacccctattgttagttgccatcattgagttgggcactctagcgagactgccggtaataaaccggaggaaggtggggatgacgtcaaatcatcatgccccttatgacctgggctacacacgtgctacaatggctggtacaacgagtcgcgagtcggtgacggcaagctaatctcttaaagccagtctcagttcggattgtaggctgcaactcgcctacatgaagtcggaatcgctagtaatcgcggatcagcacgtcgcggtgaatacgttcccgggccttgtacacaccgcccgtcacaccacgagagtttgtaacacccgaagtcggtgaggtaaccgtaaggagccagccgcctaaggtgggatagatgattggggtgaagtcgtaacaaggtagccgtatcggaaggtgcggctggatcacctcctttctaaggaa

SEQ ID NO: 124 - DNA, 1552 bp, Streptococcus mitis; identical to SEQ ID NO: 122
  misc_feature: Streptococcus mitis R 360

SEQ ID NO: 125 - DNA, 1430 bp, Streptococcus mitis
  misc_feature: Streptococcus mitis R 360
aggatgaacgctggcggcgtgcctaatacatgcaagtagaacgctgaagcttggtgcttgcaccgagcggatgagttgcgaacgggtgagtaacgcgtaggtaacctgcctcttagcgggggataactattggaaacgatagctaataccgcataaaagtcgatatcgcatgatattgatttgaaaggtgcaaatgcatcactaagagatggacctgcgttgtattagctagttggtgaggtaacggctcaccaaggcgacgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgtaagagaagaacgagtgtgagagtggaaagttcacactgtgacggtaacttaccagaaagggacggctaactacgtgccagcagccgcggtaatacgtaggtcccgagcgttatccggatttattgggcgtaaagcgagcgcaggcggttagataagtctgaagttaaaggctgtggcttaaccatagtacgctttggaaactgtttaacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacaggattagataccctggtagtccacgccgtaaacgatgagtgctaggtgttgggtcctttccgggactcagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccctctgaccgctctagagatagagttttccttcgggacagaggtgacaggtggtgcatggttgtcgtcagctcgtgtcgtgagatgttgggttaagtcccgcaacgagcgcaacccctattgttagttgccatcattgagttgggcactctagcgagactgccggtaataaaccggaggaaggtggggatgacgtcaaatcatcatgccccttatgacctgggctacacacgtgctacaatggctggtacaacgagtcgcgagtcggtgacggcaagctaatctcttaaagccagtctcagttcggattgtaggctgcaactcgcctacatgaagtcggaatcgctagtaatcgcggatcagcacgtcgcggtgaatacgttcccgggccttgtacacaccgcccgtcacaccacgagagtttgtaacacccgaagtcggtgaggtaacc

SEQ ID NO: 126 - DNA, 1461 bp, Turicella otitidis
  misc_feature: Turicella otitidis 234/92
  misc_feature (10)..(10): n is a, c, g, or t
  misc_feature (125)..(125): n is a, c, g, or t
  misc_feature (871)..(871): n is a, c, g, or t
  misc_feature (1416)..(1416): n is a, c, g, or t
  misc_feature (1431)..(1431): n is a, c, g, or t
  misc_feature (1441)..(1441): n is a, c, g, or t
cgaacgctgncggcgtgcttaacacatgcaagtcgaacggaaaggcctactttcccttgtggattgcgggtgctcgagtggcgaacgggtgagtaacacgtgagtgatctgcccccaacttgggnataagcctgggaaactgggtctaattcccgataggactgcagggtggtgcctgtggtggaaaacgattttctagtggttggggatgagctcgcggcctatcagcttgttggtggggtaatggcctaccaaggcggcgacgggtagccggcctgagagggtggacggccacattgggactgagatacggcccagactcctacgggaggcagcagtggggaatattgcacaatgggcggaagcctgatgcagcgacgccgcgtgggggatgacggccttcgggttgtaaactcctttcgaccgcgaggaagccgcctggttggaagggtggtgacggtagtggtagaagaagcaccggctaactacgtgccagcagccgcggtaatacgtagggtgcgagcgttgtccggatttactgggcgtaaagagctcgtaggtggcttgtcgcgtcgtctgtgaaagtctggggcttaactccgggtgtgcaggcgatacgggctggcttgagtgctgtaggggagactggaattcctggtgtagcggtggaatgcgcagatatcaggaggaacaccgatggcgaaggcaggtctctgggcagtcactgacgctgaggagcgagagcatgggtagcgaacaggattagataccctggtagtccatgctgtaaacggtgggcgctaggtgtggggactttccatggtttccgtgtcctagctaacgcgttaagcgccccgcctggggagtacgnccgcaaggctaaaactcaaaggaattgacgggggcccgcacaagcggcggagcatgtggattaattcgatgcaacgcgaagaaccttacctgggtttgacatgcactagattaggcgagagatcgtctgtccctttgtggctggtgtgcaggtggtgcatggttgtcgtcagctcgtgtcgtgagatgttgggttaagtcccgcaacgagcgtaacccttgtcttatgttgccagcacgttgtggtggggactcgtgagagactgccggggtgaactcggaggaaggtggggatgacgtcaaatcatcatgccccttatgtccagggcttcacacatgctacaatggccggtacagtaggttgcgagaccgtgaggtggagctaatcctgtaaagctggtcgtagttcggattggggtctgcaactcgaccccatgaagtcggagtcgctagtaatcgcagatcaccattgctgcggtgaatacgttctcgggccttgtacacaccgcccgtcacgtcatgaaagttggtaacacccgaagccgnttgctccacttagtntggggtggtntcgaaggtgggatcggcgat

SEQ ID NO: 127 - DNA, 1552 bp, Turicella otitidis
  misc_feature: Turicella otitidis 234/92
tgttgatggagagtttgatcctggctcaggacgaacgctggcggcgtgcttaacacatgcaagtcgaacggaaaggcctgctttcccttgtggattgcgggtgctcgagtggcgaacgggtgagtaacacgtgagtgatctgcccccaacttgggtataagcctgggaaactgggtctaattcccgataggactgcagggtggtgcctgtggtggaaaacgattttctagtggttggggatgagctcgcggcctatcagcttgttggtggggtaatggcctaccaaggcggcgacgggtagccggcctgagagggtggacggccacattgggactgagatacggcccagactcctacgggaggcagcagtggggaatattgcacaatgggcggaagcctgatgcagcgacgccgcgtgggggatgacggccttcgggttgtaaactcctttcgaccgcgaggaagccgcctggttggaagggtggtgacggtagtggtagaagaagcaccggctaactacgtgccagcagccgcggtaatacgtagggtgcgagcgttgtccggatttactgggcgtaaagagctcgtaggtggcttgtcgcgtcgtctgtgaaagtctggggcttaactccgggtgtgcaggcgatacgggctggcttgagtgctgtaggggagactggaattcctggtgtagcggtggaatgcgcagatatcaggaggaacaccgatggcgaaggcaggtctctgggcagtcactgacgctgaggagcgagagcatgggtagcgaacaggattagataccctggtagtccatgctgtaaacggtgggcgctaggtgtggggactttccatggtttccgtgtcgtagctaacgcgttaagcgccccgcctggggagtacggccgcaaggctaaaactcaaaggaattgacgggggcccgcacaagcggcggagcatgtggattaattcgatgcaacgcgaagaaccttacctgggtttgacatgcactagattaggcgagagatcgtctgtccctttgtggctggtgtgcaggtggtgcatggttgtcgtcagctcgtgtcgtgagatgttgggttaagtcccgcaacgagcgtaacccttgtcttatgttgccagcacgttgtggtggggactcgtgagagactgccggggtgaactcggaggaaggtggggatgacgtcaaatcatcatgccccttatgtccagggcttcacacatgctacaatggccggtacagtaggttgcgagaccgtgaggtggagctaatcctgtaaagctggtcgtagttcggattggggtctgcaactcgaccccatgaagtcggagtcgctagtaatcgcagatcagcaacgctgcggtgaatacgttcccgggccttgtacacaccgcccgtcacgtcatgaaagttggtaacacccgaagccgcttgctccactttgtgtggggtggtgtcgaaggtgggatcggcgattgggacgaagtcgtaacaaggtagccgtaccggaaggtgcggctggatcacctcctttct

SEQ ID NO: 128 - DNA, 1552 bp, Turicella otitidis; identical to SEQ ID NO: 127
  misc_feature: Turicella otitidis 234/92

SEQ ID NO: 129 - DNA, 1569 bp, Turicella otitidis
  misc_feature: Turicella otitidis 234/92
gtgtgggtggttgttgatggagagtttgatcctggctcaggacgaacgctggcggcgtgcttaacacatgcaagtcgaacggaaaggcctgctttcccttgtggattgcgggtgctcgagtggcgaacgggtgagtaacacgtgagtgatctgcccccaacttgggtataagcctgggaaactgggtctaattcccgataggactgcagggtggtgcctgtggtggaaaacgattttctagtggttggggatgagctcgcggcctatcagcttgttggtggggtaatggcctaccaaggcggcgacgggtagccggcctgagagggtggacggccacattgggactgagatacggcccagactcctacgggaggcagcagtggggaatattgcacaatgggcggaagcctgatgcagcgacgccgcgtgggggatgacggccttcgggttgtaaactcctttcgaccgcgaggaagccgcctggttggaagggtggtgacggtagtggtagaagaagcaccggctaactacgtgccagcagccgcggtaatacgtagggtgcgagcgttgtccggatttactgggcgtaaagagctcgtaggtggcttgtcgcgtcgtctgtgaaagtctggggcttaactccgggtgtgcaggcgatacgggctggcttgagtgctgtaggggagactggaattcctggtgtagcggtggaatgcgcagatatcaggaggaacaccgatggcgaaggcaggtctctgggcagtcactgacgctgaggagcgagagcatgggtagcgaacaggattagataccctggtagtccatgctgtaaacggtgggcgctaggtgtggggactttccatggtttccgtgtcgtagctaacgcgttaagcgccccgcctggggagtacggccgcaaggctaaaactcaaaggaattgacgggggcccgcacaagcggcggagcatgtggattaattcgatgcaacgcgaagaaccttacctgggtttgacatgcactagattaggcgagagatcgtctgtccctttgtggctggtgtgcaggtggtgcatggttgtcgtcagctcgtgtcgtgagatgttgggttaagtcccgcaacgagcgtaacccttgtcttatgttgccagcacgttgtggtggggactcgtgagagactgccggggtgaactcggaggaaggtggggatgacgtcaaatcatcatgccccttatgtccagggcttcacacatgctacaatggccggtacagtaggttgcgagaccgtgaggtggagctaatcctgtaaagctggtcgtagttcggattggggtctgcaactcgaccccatgaagtcggagtcgctagtaatcgcagatcagcaacgctgcggtgaatacgttcccgggccttgtacacaccgcccgtcacgtcatgaaagttggtaacacccgaagccgcttgctccactttgtgtggggtggtgtcgaaggtgggatcggcgattgggacgaagtcgtaacaaggtagccgtaccggaaggtgcggctggatcacctcctttctaaggag

SEQ ID NO: 130 - DNA, 1569 bp, Turicella otitidis; identical to SEQ ID NO: 129
  misc_feature: Turicella otitidis 234/92